(19) United States
(12) Patent Application Publication
(10) Pub. No.: US 2026/0240642 A1
(43) Pub. Date: Aug. 20, 2026

(54) MEDICAL IMPLANT DELIVERY SYSTEM AND RELATED METHODS

(71) Applicants: Smith & Nephew, Inc., Memphis, TN (US); Smith & Nephew Asia Pacific Pte. Limited, Singapore (SG); Smith & Nephew Orthopaedics AG, Zug (CH)

(72) Inventors: John Prudden, Manchester, MA (US); Luis Alejandro Miranda Cruz, Heridia (CR); Kevin M. Falco, Pepperell, MA (US); Elias Milad Sibani Mora, Alajuela (CR); Peter J. Cashmore, Durham, NH (US); Matthew D. Cunningham, Lakeville, MA (US); Brendan Jablonski, Boston, MA (US); Carolyn M. Krasniak, Malden, MA (US); Nehal N. Patel, Boston, MA (US); Dennis P. Colleran, North Attleboro, MA (US); Jay Anil Shah, Brighton, MA (US)

(73) Assignees: Smith & Nephew, Inc., Memphis, TN (US); Smith & Nephew Asia Pacific Pte. Limited, Singapore (SG); Smith & Nephew Orthopaedics AG, Zug (CH)

(21) Appl. No.: 19/643,151

(22) Filed: Apr. 9, 2026

Related U.S. Application Data
(63) Continuation of application No. PCT/US2024/052835, filed on Oct. 24, 2024.
(60) Provisional application No. 63/593,322, filed on Oct. 26, 2023.
(60) Provisional application No. 63/594,068, filed on Oct. 30, 2023.
(60) Provisional application No. 63/564,224, filed on Mar. 12, 2024.

(51) Int. Cl. A61F 2/08

(52) U.S. Cl. CPC A61F 2/0811; A61F 2002/0817; A61F 2002/0876

(57) ABSTRACT

An implant delivery system including a fixation member delivery system having a handle, an elongate shaft, a first elongate member coupled with the elongate shaft, and a second elongate member fixed relative to the handle. Cyclical actuation of the elongate shaft and the first elongate member may incrementally move a plurality of fixation members distally. The first elongate member may include a plurality of first engagement members disposed along the first elongate member with a distal end region of each of the one or more of the first engagement members configured to engage with one fixation member of the plurality of fixation members. The second elongate member may include a plurality of second engagement members disposed along the second elongate member with a distal end region of each of one or more of the second engagement members configured to engage with one fixation member of the plurality of fixation members.

### CROSS REFERENCE TO RELATED APPLICATIONS

This application continuation of International Application No. PCT/US 2024/052835, filed Oct. 24, 2024, which claims priority to U.S. Provisional Application No. 63/593,322 filed Oct. 26, 2023, U.S. Provisional Application No. 63/594,068 filed Oct. 30, 2023, and U.S. Provisional Application No. 63/564,224 filed Mar. 12, 2024, each of which is herein incorporated by reference in its entirety.

### TECHNICAL FIELD

The present disclosure pertains generally, but not by way of limitation, to orthopedic implants and methods of treatment. More particularly, the present disclosure relates to implant fixation delivery systems for orthopedic treatments.

### BACKGROUND

With its complexity, range of motion and extensive use, a common soft tissue injury is damage to the rotator cuff or rotator cuff tendons. Damage to the rotator cuff is a potentially serious medical condition that may occur during hyperextension, from an acute traumatic tear or from overuse of the joint. Current procedures for treatment of a torn tendon include affixing a biocompatible implant over the torn tendon. There is an ongoing need to deliver and adequately secure medical implants during an arthroscopic procedure in order to treat injuries to the rotator cuff, rotator cuff tendons, or other soft tissue or tendon injuries throughout a body.

### BRIEF SUMMARY

This disclosure provides design, material, manufacturing method, and use alternatives for medical devices. An example fixation member delivery system configured to deliver a plurality of fixation members may include a handle, an elongate shaft configured to adjust along a longitudinal axis relative to the handle, a first elongate member coupled with the elongate shaft, the first elongate member including a plurality of first engagement members disposed along the first elongate member with a distal end region of each of one or more of the first engagement members configured to engage with one fixation member of the plurality of fixation members, and a second elongate member fixed relative to the handle, the second elongate member including a plurality of second engagement members disposed along the second elongate member with a distal end region of each of one or more of the second engagement members configured to engage with one fixation member of the plurality of fixation members, wherein cyclical actuation of the elongate shaft and the first elongate member is configured to incrementally move the plurality of fixation members distally.

Alternatively or additionally to any of the configurations above, a proximal end of the second elongate member may be engaged with the handle.

Alternatively or additionally to any of the configurations above, the delivery system may further include an outer shaft extending distally from the handle, the outer shaft defining a shaft lumen, wherein the elongate shaft, the first elongate member, and the second elongate member extend along the shaft lumen.

Alternatively or additionally to any of the configurations above, the outer shaft may have an oval cross-section.

Alternatively or additionally to any of the configurations above, the delivery system may further include a filler component extending along the shaft lumen, wherein the filler component defines a fixation member lumen configured to receive the plurality of fixation members.

Alternatively or additionally to any of the configurations above, the filler component may define a channel in communication with the fixation lumen and configured to receive the first elongate member.

Alternatively or additionally to any of the configurations above, the channel may be configured to permit longitudinal movement of the first elongate member and the plurality of first engagement members.

Alternatively or additionally to any of the configurations above, the filler component may define a channel in communication with the fixation lumen and the channel may be configured to receive and fix the second elongate member relative to the filler component.

Alternatively or additionally to any of the configurations above, the filler component may define a recess configured to receive a second engagement member of the plurality of second engagement members when the second engagement member flexes laterally outward.

Alternatively or additionally to any of the configurations above, the first elongate member may define an opening configured to receive a first engagement member of the plurality of first engagement members when the first engagement member flexes laterally outward.

Alternatively or additionally to any of the configurations above, the delivery system further includes an upper insert and a lower insert positioned within the shaft lumen proximate a distal end of the outer shaft.

Alternatively or additionally to any of the configurations above, the upper insert includes a notch configured to receive a portion of the first elongate member therein, and/or the lower insert includes a notch configured to receive a portion of the second elongate member therein.

Alternatively or additionally to any of the configurations above, the outer shaft is detachable from the handle via a coupling interface.

Alternatively or additionally to any of the configurations above, the coupling interface includes a locking mechanism for selectively locking axial movement of the first elongate member relative to the outer shaft.

Alternatively or additionally to any of the configurations above, the delivery system may include a push rod extending distally from the handle and configured to deploy the plurality of fixation members from a first plane, wherein the first elongate member is configured to advance the plurality of fixation members distally along a second plane.

Alternatively or additionally to any of the configurations above, the delivery system may include a spring configured to engage a distal most fixation member of the plurality of fixation members and move the distal most fixation member from the second plane to the first plane.

In another example, a fixation member delivery system configured to deliver a plurality of fixation members may include an elongate shaft configured to adjust along a longitudinal axis, a first elongate member configured to move with the elongate shaft as the elongate shaft adjusts along the longitudinal axis, the first elongate member including a plurality of flexible first engagement members disposed along the first elongate member with a distal end region of each of one or more first engagement members configured to engage one fixation member of the plurality of fixation members, and a second elongate member fixed relative to the longitudinal axis, the second elongate member including a plurality of flexible second engagement members disposed along the second elongate member with a distal end region of each of one or more second engagement members configured to engage one fixation member of the plurality of fixation members, wherein the elongate shaft and the first elongate member are configured to advance distally relative to the second elongate member and cause distal end regions of the first engagement members to engage a proximal end of a fixation member of the plurality of fixation members and advance the fixation member distally over a second engagement member of the plurality of second engagement members.

Alternatively or additionally to any of the configurations above, the elongate shaft and the first elongate member may be configured to advance proximally relative to the second elongate member and the distal end region of the second engagement members of the plurality of second engagement members may engage a proximal end of a fixation member to limit proximal movement of the fixation member.

Alternatively or additionally to any of the configurations above, a distal most second engagement member of the plurality of second engagement members may be configured to extend distal of a distal most fixation member of the plurality of fixation members to prevent inadvertent deployment of the distal most fixation member.

Alternatively or additionally to any of the configurations above, a distal most first engagement member of the plurality of first engagement members may include a distal fork configuration configured to receive a second engagement member of the plurality of second engagement members.

Alternatively or additionally to any of the configurations above, the delivery system may include a filler component extending along the first elongate member and the second elongate member, wherein the filler component is configured fix the second elongate member relative to the longitudinal axis.

In another example, a fixation member delivery system configured to deliver a plurality of fixation members may include a handle, an outer shaft extending distally from the handle, an elongate shaft configured to reciprocate along a longitudinal axis relative to the handle, a first elongate member coupled with the elongate shaft and including a plurality of first engagement members disposed along the first elongate member with a distal end region of each of one or more of the first engagement members configured to engage with one fixation member of the plurality of fixation members, a first filler component extending along the shaft lumen, and a second filler component extending along the shaft lumen. The second filler component includes a plurality of openings and a corresponding second engagement member disposed therewithin. Each of the second engagement members is configured to engage with one fixation member of the plurality of fixation members to prevent proximal retraction thereof. Cyclical actuation of the elongate shaft and the first elongate member is configured to incrementally move the plurality of fixation members distally.

Alternatively or additionally to any of the configurations above, the plurality of openings are uniformly spaced along the second filler component.

Alternatively or additionally to any of the configurations above, the first filler component and the second filler component are formed of a non-thermally conductive polymeric material.

In another example, a method of deploying a fixation member of a plurality of fixation members from a fixation member delivery system may include advancing the plurality of fixation members distally by advancing a first elongate member distally relative to a second elongate member, wherein each of one or more fixation members of the plurality of fixation members engages and flexes an engagement member of a plurality of engagement members extending from the second elongate member, deploying a distal most fixation member of the plurality of fixation members, and advancing the first elongate member proximally relative to the second elongate member, wherein each of two or more of the plurality of engagement members extending from the second elongate member engage a fixation member of the plurality of fixation members to limit proximal movement of the fixation member as the first elongate member is advanced proximally.

Alternatively or additionally to any of the configurations above, the fixation members may be configured to advance distally through a filler component extending along the first elongate member and the second elongate member.

Alternatively or additionally to any of the configurations above, wherein the fixation member comprises an interior prong and a planar proximal surface that is perpendicular to a plane along which the fixation member advances distally.

The above summary of some configurations is not intended to describe each disclosed configuration or every implementation of the present disclosure. The Figures, and Detailed Description, which follow, more particularly exemplify these configurations.

### BRIEF DESCRIPTION OF THE DRAWINGS

The disclosure may be more completely understood in consideration of the following detailed description in connection with the accompanying drawings, in which:

FIG. 1A schematically depicts an illustrative implant delivery system positioned within a shoulder of a patient;

FIG. 1B schematically depicts the illustrative implant delivery system depicted in FIG. 1 with an outer delivery sheath having been removed;

FIG. 2 schematically depicts a shoulder including a head of the humerus mating with the glenoid fossa of the scapula at a glenohumeral joint and an illustrative implant affixed to a tendon;

FIG. 3 schematically depicts a side view of an illustrative implant delivery system;

FIG. 4 schematically depicts a cross-section view of the illustrative implant delivery system depicted in FIG. 3;

FIG. 5A schematically depicts a perspective view of a distal end of the illustrative implant delivery system depicted in FIG. 3 with a delivery sheath applied thereto;

FIG. 5B schematically depicts an exploded perspective view of the implant delivery system depicted in FIG. 5A;

FIG. 5C schematically depicts a perspective view of the distal end of the illustrative implant delivery system depicted in FIG. 3;

FIG. 6 schematically depicts a cross-section view of the distal end of the illustrative implant delivery system depicted in FIG. 3;

FIG. 7 schematically depicts a cross-section view taken at an axial location along a shaft assembly of the illustrative implant delivery system depicted in FIG. 3;

FIG. 8 schematically depicts an exploded perspective view of a portion of an actuation assembly of the illustrative implant delivery system depicted in FIG. 3;

FIG. 9 schematically depicts a perspective view of a portion of an actuation assembly of the illustrative implant delivery system depicted in FIG. 3;

FIG. 10 schematically depicts a perspective view of a portion of a shaft assembly of the illustrative implant delivery system depicted in FIG. 3;

FIG. 11 schematically depicts a perspective view of a portion of a shaft assembly of the illustrative implant delivery system depicted in FIG. 3;

FIGS. 12-16 schematically depict an illustrative technique for using an implant delivery system;

FIG. 17 schematically depicts a cross-section view of an illustrative implant delivery system;

FIG. 18 schematically depicts a cross-section view of a portion of the illustrative implant delivery system depicted in FIG. 17;

FIG. 19 schematically depicts a cross-section view of a shaft assembly of the illustrative implant delivery system depicted in FIG. 17;

FIG. 20 schematically depicts a cross-section view taken at an axial location along a shaft assembly of the illustrative implant delivery system depicted in FIG. 17;

FIG. 21 schematically depicts a side view of a portion of an actuation assembly of the illustrative implant delivery system depicted in FIG. 17;

FIG. 22 schematically depicts a perspective view of a portion of an actuation assembly of the illustrative implant delivery system depicted in FIG. 17;

FIG. 23 schematically depicts a perspective view of an illustrative fixation member;

FIG. 24 schematically depicts a side view of the illustrative fixation member depicted in FIG. 23;

FIG. 25 schematically depicts a distal end perspective view of the illustrative fixation member depicted in FIG. 23;

FIG. 26 schematically depicts a perspective cross-section view taken along a longitudinal axis of a shaft assembly of another illustrative implant delivery system;

FIG. 27 schematically depicts an exploded perspective view of components of the shaft assembly of FIG. 26;

FIG. 28 schematically depicts a cross-section view taken along a longitudinal axis of the shaft assembly of FIG. 26;

FIG. 29 schematically depicts the distal end region of the shaft assembly of FIG. in a first actuation position;

FIG. 30 schematically depicts the distal end region of the shaft assembly of FIG. is a second actuation position;

FIG. 31A schematically depicts a perspective view of an alternative distal end cap for a shaft assembly of another illustrated implant delivery system;

FIG. 31B schematically depicts a top view of the distal end cap of FIG. 31A;

FIG. 31C schematically depicts a side view of the distal end cap of FIG. 31A;

FIG. 32 schematically depicts a longitudinal cross-section view of the distal end cap of FIG. 31A;

FIG. 33 schematically depicts a transverse cross-section view of the distal end cap taken along line 33-33 of FIG. 32;

FIG. 34 schematically depicts a perspective view of a shaft assembly of another illustrative implant delivery system;

FIG. 35 schematically depicts a cross-section view taken along a longitudinal axis of the shaft assembly of FIG. 34;

FIG. 36A schematically depicts a transverse cross-section view taken through the shaft assembly of FIG. 34;

FIG. 36B schematically depicts a transverse cross-section view of an alternative configuration of the shaft assembly of FIG. 34;

FIG. 37 schematically depicts a perspective view of a shaft assembly of another illustrative implant delivery system;

FIG. 38 schematically depicts a perspective bottom view of a component of the shaft assembly of FIG. 37;

FIG. 39 schematically depicts a perspective top view of the component of FIG. 38;

FIG. 40 schematically depicts a top view of a portion of the component of FIGS. 38-39;

FIG. 41 schematically depicts a cross-section view of the portion of the component shown in FIG. 40 taken along line 41-41;

FIG. 42 schematically depicts a perspective view of components of the shaft assembly of FIG. 37;

FIG. 43 schematically depicts a perspective view of a distal end region of a component of the shaft assembly of FIG. 42;

FIG. 44 schematically depicts a perspective view of a shaft assembly of another illustrative implant delivery system;

FIG. 45 schematically depicts a perspective view of components of the shaft assembly of FIG. 44;

FIG. 46 schematically depicts an alternative implant delivery system having an attachable/detachable shaft assembly;

FIG. 47 schematically depicts a cross-section view of a coupling interface of the attachable/detachable shaft assembly of FIG. 46;

FIG. 48 schematically depicts a partial cross-section view of the implant delivery system of FIG. 46;

FIG. 49A schematically depicts a perspective view of the attachable/detachable shaft assembly of FIG. 46 in a locked position;

FIG. 49B schematically depicts a perspective view of the attachable/detachable shaft assembly of FIG. 46 in an unlocked position;

FIG. 50 schematically depicts a cross-section view of the coupling interface of the attachable/detachable shaft assembly of FIG. 46;

FIG. 51 schematically depicts a cross-section view of a handle assembly of another illustrative implant delivery system;

FIG. 52 schematically depicts a perspective view of an attachable/detachable shaft assembly for coupling to the handle assembly of FIG. 51;

FIG. 53 schematically depicts a perspective view of another attachable/detachable shaft assembly for coupling to the handle assembly of FIG. 51;

FIG. 54 schematically depicts a perspective view of another implant delivery system having an alternative attachable shaft assembly;

FIG. 55 schematically depicts a partial cross-section view and partial side view of a distal end of an illustrative implant delivery system;

FIG. 56 schematically depicts a perspective view of a portion of an actuation assembly of an illustrative implant delivery system; and

FIG. 57 schematically depicts a perspective view of a distal end of an illustrative implant delivery system;

While the disclosure is amenable to various modifications and alternative forms, specifics thereof have been shown by way of example in the drawings and will be described in detail. The intention, however, is not to limit the disclosure to the particular configurations described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “a configuration”, “some configurations”, “other configuration”, etc., indicate that the configuration described may include one or more particular features, structures, and/or characteristics. However, such recitations do not necessarily mean that all configurations include the particular features, structures, and/or characteristics. Additionally, when particular features, structures, and/or characteristics are described in connection with one configuration, such features, structures, and/or characteristics may also be used connection with other configurations whether or not explicitly described unless clearly stated to the contrary.

The following detailed description should be read with reference to the drawings in which similar elements in different drawings are numbered the same. The drawings, which are not necessarily to scale, depict illustrative configurations and are not intended to limit the scope of the disclosure.

With its complexity, range of motion and extensive use, a common soft tissue injury is damage to the rotator cuff or rotator cuff tendons. Damage to the rotator cuff is a potentially serious medical condition that may occur during hyperextension, from an acute traumatic tear or from overuse of the joint. Current repair procedures may attempt to alleviate impingement or make room for movement of the tendon to prevent further damage and relieve discomfort. An accepted treatment for rotator cuff tears may include reattaching the torn tendon to the humeral head using sutures or anchors. Additionally, in treating rotator cuff tears, an accepted practice may also include the placement of a scaffold over the repaired tendon to mechanically reinforce the repaired tendon or promote tissue growth for natural repair. The scaffold may be secured to the tendon using one or more, or a plurality of, fixation members, such as staples or other anchors. Therefore, there is an ongoing need to deliver and adequately secure medical implants during an arthroscopic procedure in order to treat injuries to the rotator cuff, rotator cuff tendons, and/or other soft tissue or tendon injuries throughout a body (e.g., a human body or a body of other animals).

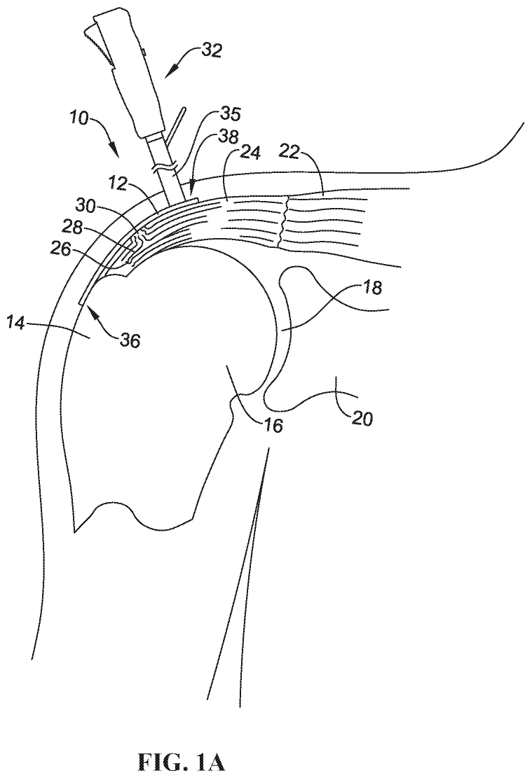

FIG. 1A schematically illustrates a cross-sectional view of a shoulder 10 including an example implant 12. The shoulder 10 further shows a head 14 of the humerus 16 mating with a glenoid fossa 18 of the scapula 20. The glenoid fossa 18 includes a shallow depression in the scapula 20. A supraspinatus tendon 22 is also shown. These muscles (along with others) control the movement of the humerus 16 relative to the scapula 20. A distal tendon 24 of the supraspinatus tendon 22 meets the humerus 16 at an insertion point 26.

In FIG. 1A, the tendon 24 includes a damaged portion 28 located near the insertion point 26. The damaged portion 28 includes a tear 30 extending partially through the tendon 24. The tear 30 may be referred to as a partial thickness tear. The depicted partial thickness tear 30 is on the bursal side of the tendon 24, however, the tear may also be on the opposite or articular side of the tendon 24 and/or may include internal tears to the tendon 24 not visible on either surface.

FIG. 1A further illustrates that the tendon repair implant 12 has been placed over the partial thickness tear 30. In this example, the tendon repair implant 12 is placed on the bursal side of the tendon regardless of whether the tear is on the bursal side, articular side or within the tendon. Further, the tendon repair implant 12 may overlay multiple tears.

In some instances, delivery of an implant 12 (e.g., a sheet-like implant) to a target site of a patient may require a physician to create an incision in the patient sufficient to access the target implant site. After creating this “access site,” the physician may insert an implant delivery system through the access site and position the distal end of the implant delivery system adjacent the target implant site. The physician may then manipulate the implant delivery system to deploy an implant out of a delivery sheath (not shown in FIG. 1A) adjacent the target implant site.

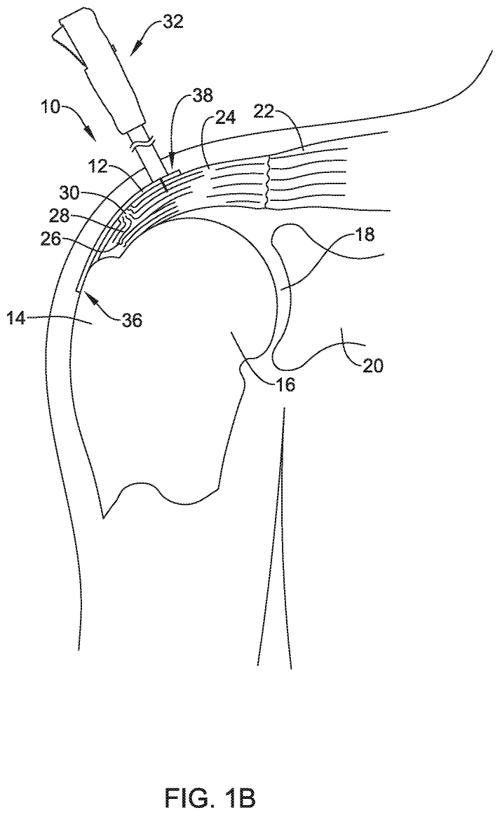

When positioning the implant 12 adjacent a target site, a clinician may orient the implant 12 such that the proximal portion 36 may be adjacent (e.g., overlaid) on a portion of the humerus 16 (e.g., on the bone), while the distal portion 38 of the implant 12 may overlay the tendon 24. Further, once the implant 12 has been placed appropriately, it may be desirable to utilize a fixation member delivery system 32 (e.g., stapling instrument) to insert fixation members 34 (e.g., staples, anchors, etc., which are not shown in FIG. 1A, but shown in FIG. 2) through the implant 12 into the tendon tissue 24 and/or the bone (e.g., the humerus 16).

FIG. 1A further illustrates that, in some examples, the delivery system 32 may include a removeable outer access sheath 35 designed to provide an atraumatic entry profile during the advancement of the delivery system 32 to the implant site. As will be described in greater detail herein, the outer access sheath 35 may cover the distal end region of the delivery system 32, thereby shielding portions of the patient's shoulder from sharp portions of the delivery system 32 as the delivery system 32 is inserted through the skin (e.g., the access site) and positioned adjacent the implant 12.

FIG. 1B schematically illustrates the delivery system 32 positioned adjacent to the implant 12. Additionally, FIG. 1B illustrates that the outer access sheath 35 (shown in FIG. 1A) has been removed from the delivery system 32. Once the delivery system 32, and outer access sheath 35 coupled thereto, has been inserted through the incision and directed to the surgical site, the outer access sheath 35 may be decoupled and removed from the delivery system 32. As described herein, removing the outer access sheath 35 from the delivery system 32 may expose portions of the delivery system 32, which may be utilized to affix the implant 12 to the humerus 16 and/or the tendon 24. For example, the implant 12 may be anchored to the humeral head using one or more bone anchors (e.g., staples) and the implant 12 may be anchored to the tendon 24 using a plurality of tendon anchors (e.g., staples) arranged around the periphery of the implant 12.

FIG. 2 schematically illustrates the cross-section of the shoulder 10 shown in FIGS. 1A-1B, whereby the implant 12 may be positioned such that the proximal portion 36 may be adjacent (e.g., overlaid) on a portion of the humerus 16 (e.g., on the bone), while the distal portion 38 of the implant 12 may overlay the tendon 24. Further, FIG. 2 illustrates the cross-section of the shoulder 10 after a clinician has utilized the delivery system 32 (not shown in FIG. 2, but shown in FIGS. 1A-1B) to insert two example fixation members 34 (e.g., staples, anchors, etc.) along the proximal portion 36 and the distal portion 38 of the implant 12. The fixation members 34 may be used to fixedly secure the implant 12 to the tendon 24. Additional fixation members 34, such as bone staples, may be used to fixedly secure the implant 12 to the humerus 16.

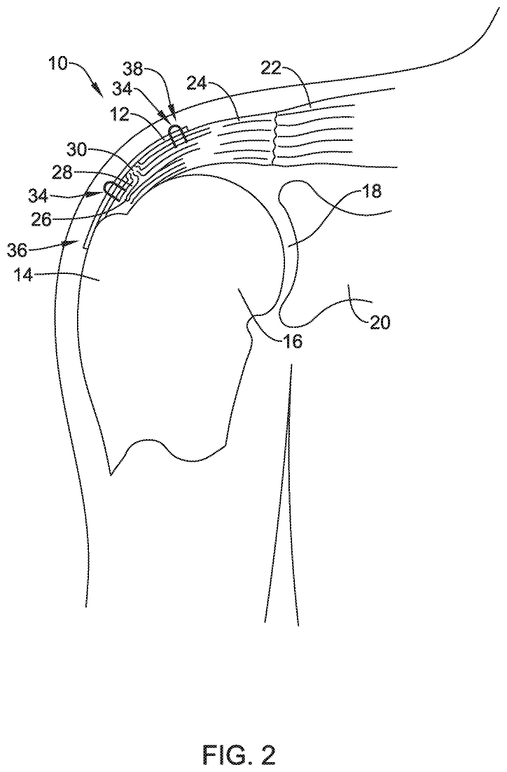

FIG. 3 illustrates an example fixation member delivery system 32, such as a tendon stapler. The delivery system 32 may include a handle 40 coupled to a shaft assembly 42. Among other components, the shaft assembly 42 may include an outer shaft 44 (e.g., an elongate outer shaft) extending in a distal direction from the handle 40. The outer shaft 44 may include one or more tines 46 extending away from the distal end of the outer shaft 44. The tines 46 may extend parallel to a central longitudinal axis L of the outer shaft 44 and/or the shaft assembly 42. It can be appreciated, that while not shown in FIG. 3, a fixation member actuation assembly may be positioned, entirely or at least partially, within a lumen (e.g., a shaft lumen) of or otherwise defined by the outer shaft 44. The fixation member actuation assembly may include or may be coupled with an actuation shaft (e.g., an elongate shaft) which extends within the outer shaft 44 and into the handle 40. A configuration of the actuation shaft within the handle 40 will be further discussed herein.

The handle 40 may include a housing 48 having a first housing member 48a, a second housing member (not shown in FIG. 3), and/or one or more other suitable housing members. The first housing member 48a and the second housing member may be designed to mate with one another in a “clam shell” configuration and/or other suitable configuration. The two or more housing member components may be coupled to one another via any suitable connection type including, but not limited to, a snap connection, screw connections, ultrasonic welding, and/or other suitable types of connections. The handle 40 may include, and the housing members thereof may enclose or house, one or more of a variety of components which are designed to manipulate the fixation member actuation assembly located in the outer shaft 44.

FIG. 3 further illustrates that the handle 40 may include an actuation mechanism or actuator 50 (e.g., a lever, trigger, and/or other suitable actuation mechanism). In some configuration, the handle 40 may include a second actuation mechanism for use with the actuator 50 (e.g., a first actuation mechanism may facilitate advancing fixation members along the shaft assembly 42 and a second actuation mechanism may facilitate deploying a distal-most fixation member, but the actuation mechanisms may be utilized for other suitable purposes). While the actuator 50 may be illustrated as including a lever, it is noted that other forms of the actuator 50 may be utilized for the actuation mechanism to manipulate the fixation member actuation assembly during use including, but not limited to a button, a dial, a slider or slide mechanism, and/or other suitable configurations of actuators. It can be appreciated that the handle 40 may be designed such that a clinician may grasp the handle 40 with one hand, actuate the one or more actuation mechanism(s) with the one hand, and manipulate the delivery system 32 with the one hand.

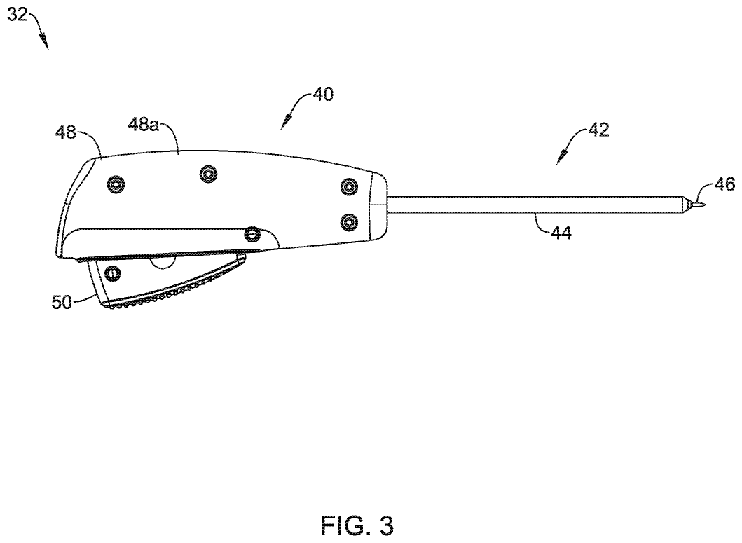

FIG. 4 schematically illustrates a cross-section of the inner components of the delivery system 32, with the first housing member 48a removed and depicting a second housing member 48b of the handle 40. FIG. 4 illustrates the actuator 50, which may be coupled with a linkage assembly 52. The linkage assembly 52 may be further coupled to an elongate shaft 54 (e.g., a push rod and/or other suitable elongate shaft) configured to longitudinally adjust along the longitudinal axis L relative to the handle 40 (e.g., relative to the housing members 48a, 48b of the handle 40). In some examples, the elongate shaft 54 may be coupled with an elongate member 56, where the elongate member 56 may be configured adjust longitudinally with the elongate shaft 54 in response to actuation of the actuator 50 and relative to the housing 48 and/or the outer shaft 44. In some configurations, the elongate shaft 54 and the elongate member(s) 56 (e.g., a first elongate member and a second elongate member) may extend along the lumen of the outer shaft 44.

The linkage assembly 52 may have any suitable linkage configuration configured to transfer adjustment of the actuator 50 to linear adjustment of the elongate shaft 54. In one example configuration, as depicted in FIG. 4, the linkage assembly may include a first linkage 52a of or coupled to the actuator 50, a second linkage 52b coupled with the first linkage 52a and a third linkage 52c, where the third linkage 52c may be coupled with the elongate shaft 54. As the actuator 50 is adjusted the second linkage 52b may pivot about one or more pivot points (e.g., one or more pins, protrusions, etc.) For example, the second linkage 52b may pivot about a first pivot point 58a between the first linkage 52a and the second linkage 52b, at a second pivot point 58b between the second linkage 52b and the housing 48 or other suitable component of the handle 40, and at a third pivot point 58c between the second linkage 52b and the third linkage 52c. In some examples, the actuator 50 may include a cutout or recess 53 configured to receive a portion of the linkage assembly 52 (e.g., a portion of the second linkage 52b) as the actuator 50 is adjusted, but other suitable configurations are contemplated.

In some configurations, the handle 40 may include a spring 60 configured to bias the actuator 50 to a relaxed or unactuated position, as depicted in FIG. 4. In some examples, the spring 60 may be concentrically position about the elongate shaft 54 with a first end engage with the elongate shaft 54 and/or the third linkage 52c and a second end engaged with the housing 48 and/or a component fixed relative to the housing 48. Other suitable configurations of the spring 60 and/or other suitable biasing components relative to the linkage assembly 52 and/or the actuator 50 are contemplated.

The handle 40 may include a stop component 62 configured to engage the actuator 50. In some examples, the stop component 62 may be part of the housing 48 and/or may be fixed relative to the housing 48 such that the stop component 62 may engage the actuator 50 to limit or stop movement of the actuator 50 as the actuator 50 is actuated. In one example, the stop component 62 may be configured to engage a proximal end of the actuator 50. Other suitable configurations of a stop component 62 are contemplated.

The handle 40 may include a tactile feedback component 64 configured to engage the actuator 50, the linkage assembly 52, and/or other suitable components configured to provide tactile and/or audible feedback to a user (e.g., clinician) as the actuator 50 adjusted relative to the housing 48. In one example, the handle 40 may include the tactile feedback component 64 (e.g., a tab, a flexible tab, etc.) configured to engage one or more tabs or protrusions 66 extending from the second linkage 52b, but other suitable configurations are contemplated. In some examples, the tactile feedback component 64 and the protrusion(s) 66 may be positioned relative to one another such that the tactile and/or audible feedback is associated with a full actuation of the actuator 50 and/or advancement or deployment of a fixation member, but other suitable configurations are contemplated.

The delivery system 32 may include an elongate filler component 68 (e.g., a bumper and/or other suitable filler component) configured to extend along the shaft assembly 42 (e.g., along the lumen of the outer shaft 44). In some examples, the filler component 68 may be coupled with the handle 40 via engagement between the housing 48 and the filler component 68, as depicted in FIG. 4, but other suitable configurations are contemplated. In some examples, the filler component 68 may define a lumen 70 (e.g., a fixation member lumen) configured to receive one or more of (e.g., a plurality of) fixation members 34 and/or one or more elongate members including, but not limited to, the elongate member 56 movable along the outer shaft 44.

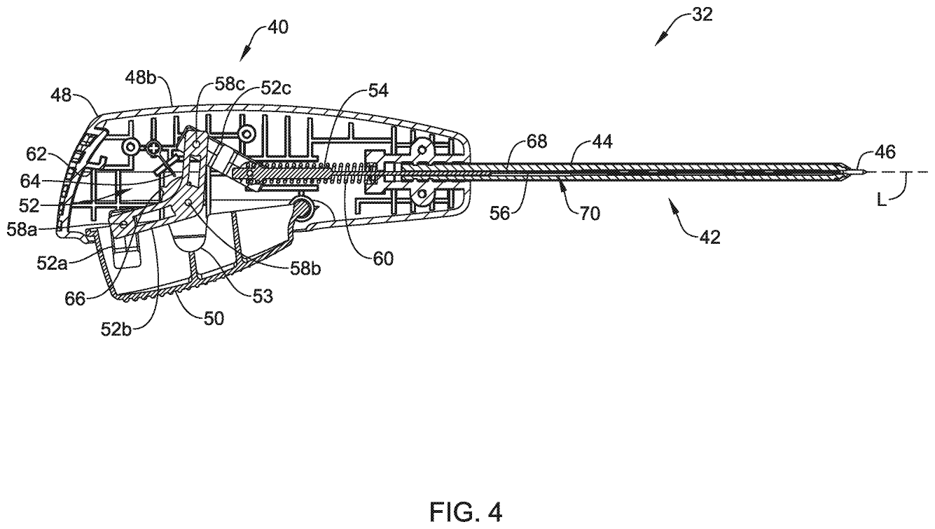

FIG. 5A schematically illustrates a distal portion of an example configuration of the delivery system 32. For example, FIG. 5A illustrates the outer access sheath 35 described above with respect to FIG. 1A assembled onto the elongate shaft 54 (not shown in FIG. 5A) of the delivery system 32. It can be appreciated that the outer access sheath 35 may include a distal end region 37 and a proximal end region 39. As illustrated in FIG. 5A, the distal end region 37 may include a tapered tip portion, but other suitable configurations are contemplated. The distal end region 37 may extend distally beyond the distal extent of the elongate shaft of the delivery system 32, such as distally beyond the tines 46. The tapered tip portion, when included, may provide the delivery system 32 with an atraumatic entry profile. In other words, the tapered tip portion of the distal end region 37 of the outer access sheath 35 may provide a tapered tissue entry profile (e.g., a gradual entry profile) which may limit trauma to the skin (or other tissue) as the distal end region of the outer access sheath 35 is inserted into a patient.

After being inserted through an access site of the patient, the outer access sheath 35 may be removed from both the delivery system 32 and the patient. For example, FIG. 5B schematically illustrates the outer access sheath 35 after being removed from the outer shaft 44 of the delivery system 32.

FIG. 5B further illustrates that the proximal end region of the access sheath 35 may include a finger loop 72 or other structure to grasp in order to separate the access sheath 35 from the outer shaft 44 of the delivery system 32. The finger loop 72 may be utilized to grip and remove the outer access sheath 35 from the outer shaft 44. For example, after being inserted through an access site of the patient and positioned adjacent to the implant site, a physician may grip the outer access sheath 35 via the finger loop 72 and subsequently remove the outer access sheath 35 from the outer shaft 44 via pulling on the finger loop 72 (which imparts a removal force sufficient to release the outer access sheath 35 from the outer shaft 44).

It can be appreciated from FIG. 5B that the outer access sheath 35 may be designed to be press-fit (e.g., snap fit) along a portion of the outer shaft 44 of the delivery system 32. In some instances, a portion of the outer access sheath 35 may not extend completely around the outer surface of the outer shaft 44. For example, one or more portions of the outer access sheath 35 may include a cross-sectional shape which is substantially semi-circular and designed to mate with the contour of the outer surface of the outer shaft 44. For example, the access sheath 35 may include a longitudinal slot extending the entire length of the access sheath 35 from the distal end region 37 of the access sheath 35 to the proximal end region 39 of the access sheath 35. Thus, the access sheath 35 may be coupled to the outer shaft 44 by laterally inserting the outer shaft 44 through the longitudinal slot and/or the access sheath 35 may be removed or decoupled from the outer shaft 44 by laterally withdrawing the outer shaft 44 through the longitudinal slot. Accordingly, the outer access sheath 35 may be designed to be press fit onto the outer surface of the outer shaft 44. It can be further appreciated that the access sheath 35 may be designed such that the force required to remove the outer access sheath 35 from the outer shaft 44 may be large enough to prevent the outer access sheath 35 from coming off the outer shaft 44 while being inserted through a tissue access site, while also being low enough that it can be removed from the outer shaft 44 after insertion into the patient.

In some instances, the access sheath 35 may include a proximal portion at the proximal end region 39 that wraps or extends around greater than 180°, but less than 360°, of the circumference of the outer shaft 44 and a distal portion at the distal end region 37 that also wraps or extends around greater than 180°, but less than 360°, of the circumference of the outer shaft 44. In some configurations, a medial portion of the access sheath 35 between the distal end region 37 and the proximal end region 39 may include a portion that also wraps or extends around greater than 180°, but less than 360°, of the circumference of the outer shaft 44, but other configurations are contemplated. Further, the medial portion extending between the proximal end region 39 and the distal end region 37 may include one or more portions that wraps around the circumference of the outer shaft 44 less than at the proximal end region 39 and the distal end region 37. For example, one or more portions of the medial portion may wrap or extend around the outer shaft 44 for 180°or less than 180°of the circumference of the outer shaft 44. Thus, the longitudinal slot along the medial portion may be wider than the longitudinal slot through the proximal and distal end regions 39, 37 of the access sheath 35.

To laterally remove the access sheath 35 from the outer shaft 44, the user may pull on the loop 72 to laterally remove the outer shaft 44 from the proximal end region 39 of the access sheath 35 while the distal end region 37 of the access sheath 35 remains wrapped around the outer shaft 44. The access sheath 35 may then be withdrawn proximally such that the distal end region 37 of the access sheath 35 slides along the outer shaft 44 proximally until the distal end region 37 of the access sheath 35 has passed through the incision to the exterior of the patient. Once exterior of the patient, the user may again apply a lateral force to decouple the access sheath 35 from the outer shaft 44 by passing the outer shaft 44 out through the longitudinal slot along the distal portion of the access sheath 35.

FIG. 5B further illustrates that the distal end region 37 of the access sheath 35 may partially cover (or in some examples, completely cover) one or more of the tines 46 which extend distally from the distal end of the outer shaft 44. Further, the one or more tines 46 may include a sharp, pointed tip portion which may be utilized to create a pilot hole at the target tissue site. However, as discussed above, it may be undesirable for the tines 46 to engage with tissue other than at the implant site. Therefore, the distal end region 37 of the outer access sheath 35 may house (e.g., nest, cover, etc.) the one or more tines 46 within the distal end region 37 of the outer access sheath 35 while passing the delivery system 32 through the access site to the implant securement site. Housing the one or more tines 46 within the distal end region 37 of the outer access sheath 35 may shield the one or more tines 46 from tissue as the delivery system 32 is advanced to the implant securement site.

FIG. 5C schematically illustrates another portion of the example configuration of the delivery system 32. In particular, FIG. 5C illustrates the shaft assembly 42 of the delivery system 32 (shown in FIG. 3). As is discussed in greater detail herein, the shaft assembly 42 may include a portion of the delivery system 32 which extends distally away from a handle 40. Additionally, as described above, FIG. 5C illustrates that the shaft assembly 42 may include the elongate outer shaft 44, which may be an elongate tubular member. The outer shaft 44 may include an outer surface and a lumen extending therein. Additionally, the distal end region of the outer shaft 44 may include a distal face 74. The distal face 74 may include a surface of the outer shaft 44 which is tapered distally toward the longitudinal axis of the outer shaft 44 or the shaft assembly 42. In some configurations, the distal face 74 may include at least a portion that is perpendicular to the longitudinal axis of the outer shaft 44 or the shaft assembly 42.

FIG. 5C illustrates that the shaft assembly 42 may include a pair of tines 46 extending distally from the distal face 74. Collectively, the pair of tines 46 may define a passage through which a fixation member (not shown in FIG. 5C) may pass through as the fixation member is deployed out of the outer shaft 44 between the tines 46. Further, the tines 46 may be designed such that the tines 46 create a pilot hole within the target site tissue. For example, after a clinician may align the distal end of the shaft assembly 42 along the implant 12, the clinician may apply a force to the outer shaft 44 such that the tines 46 pierce through the implant 12 and into the target site (e.g., tendon tissue), thereby creating a pilot hole through which a fixation member 34 (e.g., staple) may be inserted.

The tines 46 may include curved sides (e.g., concave surfaces facing the opposed tine 46 of the pair of tines) and a pointed end. In some examples, the curved sides of tines 46 may be configured to mate with curved sides of a variety of example fixation members. In different examples, the tines 46 may take various shapes, such as spikes, spears, prongs, or other shapes. Whatever shape the tines 46 may take, the tines 46 may generally have pointed distal ends for piercing tissue or bone.

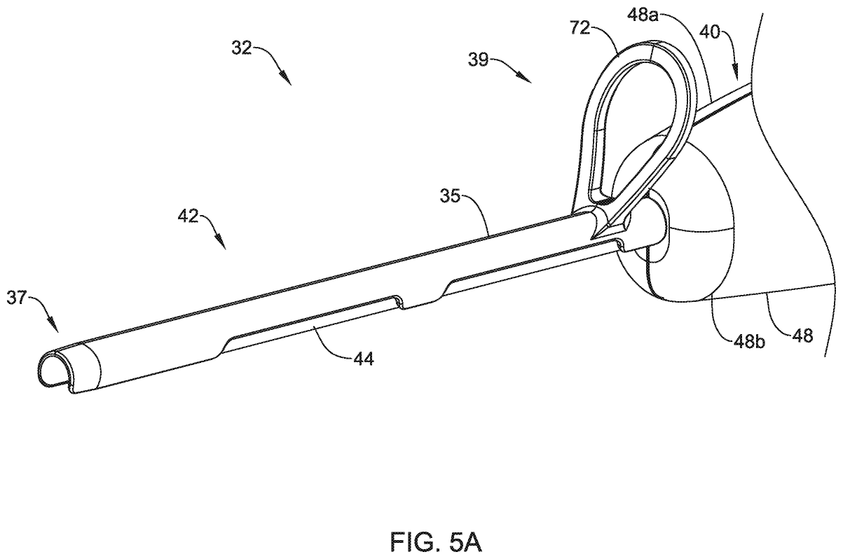

FIG. 6 schematically illustrates a view of the shaft assembly 42 shown in FIG. 5C, with the outer shaft 44 and the filler component 68 in a cross-section and the first elongate member 56a, the second elongate member 56b, and the fixation members 34 in a side view. FIG. 6 illustrates that the shaft assembly 42 may include a fixation member actuation assembly 76 positioned within the lumen of the outer shaft 44. The fixation member actuation assembly 76 may include one or more components, which collectively, work together to deploy fixation members 34 (e.g., staples) out the distal end of the shaft assembly 42.

The filler component 68 extending through the lumen of the outer shaft 44 may include one or more portions defining the lumen 70. In some examples, the filler component 68 may include a first filler component 68a and a second filler component 68b configured to entirely or at least partially define the lumen 70. Other suitable configurations are contemplated.

In some examples, the fixation member actuation assembly 76 may include a first elongate member 56a, such as a first longitudinal member or beam, and a second elongate member 56b, such as a second longitudinal member or beam. In some examples, the first elongate member 56a and the second elongate member 56b may be referred to as a first beam 56a and a second beam 56b, respectively. The first elongate member 56a and the second elongate member 56b may extend along or through the lumen 70 of the filler component 68. The first elongate member 56a may couple with the elongate shaft 54 and the second elongate member 56b may be coupled with and at least partially fixed relative to the filler component 68. In some example configurations, the first elongate member 56a may be omitted and the elongate shaft 54 be configured to move longitudinally along the second elongate member 56b and advance fixation members 34 relative to the second elongate member 56b as discussed herein with respect to the first elongate member 56a.

The first elongate member 56a may be configured to longitudinally adjust relative to the filler component 68 to advance the fixation members 34 distally and deploy a distal-most fixation member 34 through the tines 46. The second elongate member 56b may be longitudinally fixed relative to the filler component 68, the outer shaft 44, the handle 40, and/or other components of the delivery system 32 and may prevent or facilitate preventing proximal movement of the fixation members 34 as the first elongate member 56a is adjusted in the proximal direction during an actuation cycle.

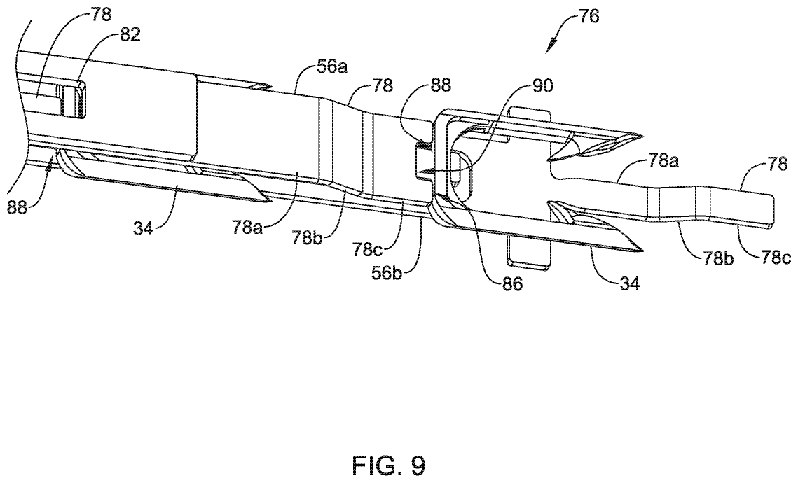

The actuation cycle (e.g., cyclical actuation) may be a cycle of movement of the first elongate member 56a, where the first elongate member 56a and the elongate shaft 54 may move in the distal direction to advance the fixation members 34 in response to actuation of the actuator 50 and move in the proximal direction to reset the engagement members 78 of the first elongate member 56a with the respect to the next sequentially positioned fixation member 34 in response to releasing the actuator 50. The actuation cycle is discussed in greater detail with respect to FIGS. 12-16. Other examples of the actuation cycle are contemplated.

The elongate members 56 (e.g., the first elongate member 56a, the second elongate member 56b, and/or other suitable elongate members) may be formed from any suitable material. For example, the elongate members 56 may be formed from one or more materials including, but not limited to, metals, polymers, alloys, 304 stainless steel, sheet metal, and/or other suitable materials. In some examples, each of the elongate members 56 may be constructed entirely or at least partially from 304 stainless steel having a raw material yield strength of 160-185 ksi per ASTM 666. Constructing the elongate members 56 from 304 stainless steel having a raw material yield strength of 160-185 ksi per ASTM 666 may provide each of the elongate members 56 with enough flexibility to allow the engagement members 78 to flex during the actuation cycle while also providing sufficient column strength to advance the fixation members 34 through an implant an into tissue.

The elongate members 56 and features thereof may be formed from any suitable manufacturing techniques. For example, the elongate members 56 and/or features thereof may be formed using one or more manufacturing techniques including, but not limited to, punching, cutting, laser-cutting, folding, molding, over molding, and/or other suitable manufacturing techniques. In some examples, the elongate members 56 and/or features thereof may be formed from a combination of punching or cutting and bending sheet metal.

The first elongate member 56a and the second elongate member 56b may have similar or different configurations. In some examples, the first elongate member 56a and the second elongate member 56b may be formed from material having a same thickness or from material having different thicknesses. In one example when the first elongate member 56a and the second elongate member 56b are formed from material having different thicknesses or otherwise include features or components having different thicknesses, the second elongate member 56b configured to be fixed relative to the outer shaft 44 may be or may have features that are less thick than the first elongate member 56a and/or features of the first elongate member 56a. Thicker material of the first elongate member 56a may facilitate increasing the rigidity of the first elongate member 56a and the ability to push and/or deploy the fixation members 34 into the implant 12 and tissue, while thinner material of the second elongate member 56b may reduce the rigidity of the second elongate member 56b to facilitate flexible components or features of the second elongate member 56b as fixation members 34 and/or other components that move longitudinally along the filler component 68 and/or other components of the shaft assembly 42.

As depicted in FIG. 6, a plurality of fixation members 34 may be oriented in longitudinal alignment with the longitudinal axis L of the outer shaft 44 or the shaft assembly 42 with distal points of the fixation members 34 pointed toward the distal end of the outer shaft 44. As shown in FIG. 6, the fixation members 34 may be spaced away from one another along the longitudinal axis L such that adjacent fixation members 34 do not directly contact one another. Further, the fixation members 34 may be positioned along the longitudinal axis L between the first elongate member 56a and the second elongate member 56b. As such, the delivery system 32 may be initially loaded with a plurality of fixation members 34, such as four or more fixation members 34, six or more fixation members 34, or eight or more fixation members 34 for sequential deployment from the delivery system 32. As discussed in greater detail herein, the fixation members 34 may be sequentially advanced out of the distal end (e.g., through the tines 46) of the outer shaft 44 as the fixation member actuation assembly 76 is manipulated via the handle 40 (not shown in FIG. 6).

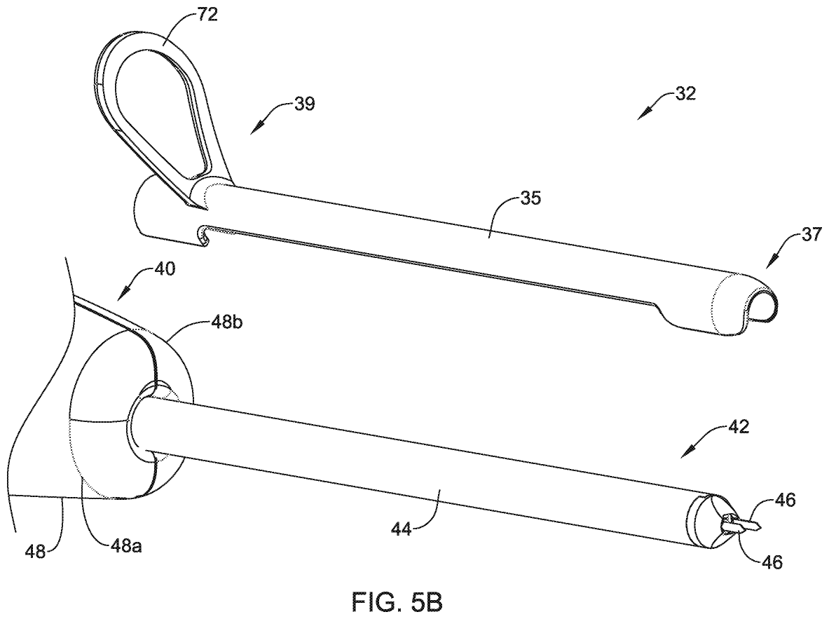

FIG. 7 depicts a cross-section taken at an axial location along the shaft assembly 42, which depicts the outer shaft 44, the filler component 68 (e.g., the first filler component portion 68a and the second filler component portion 68b), the first elongate member 56a, the second elongate member 56b, and the fixation member 34. Each of the first elongate member 56a and the second elongate member 56b may include engagement members 78 (e.g., tabs, etc.) extending toward the longitudinal axis of the outer shaft 44 or the shaft assembly 42.

The engagement members 78 may be configured to flex. For example, the engagement member may flex as the engagement members 78 of the second elongate member 56b engage the fixation members 34 during distal advancement of the fixation members 34 along the shaft assembly 42 and as the engagement members 78 of the first elongate member 56a engage the fixation members 34 when the first elongate member 56a is adjusted proximally during the actuation cycle.

The second filler component portion 68b may include one or more channels or recesses 80 in communication with the lumen 70 and aligned with and configured to receive the engagement members 78 of the second elongate member 56b as the engagement members 78 flex outward (e.g., laterally outward) from the longitudinal axis of the outer shaft 44 or the shaft assembly 42 in response to the engagement members 78 engaging the fixation members 34. In some configurations, the first elongate member 56a may define one or more channels or openings 82 in communication with the lumen 70 and aligned with and configured to receive the engagement members 78 of the first elongate member 56a as the engagement members 78 flex outward (e.g., laterally outward) from the longitudinal axis of the outer shaft 44 or the shaft assembly 42 in response to engaging the fixation members 34 as the first elongate member 56a moves in a proximal direction. In some examples, the first elongate member 56a may have a thickness greater than a thickness of the second elongate member 56b to facilitate defining the openings 82 configured to receive the engagement members 78, but other suitable configurations are contemplated. Alternatively or additionally, the first filler component portion 68a may define a recess configured to receive the engagement members 78 of the first elongate member 56a as the engagement members 78 flex outward. Other suitable configurations are contemplated.

As discussed, the filler component 68 may define the lumen 70. The lumen 70 may have any suitable configuration. In some examples, the lumen 70 may be shaped or configured (e.g., may have an oval cross-section or other shaped cross-section) to reduce or limit movement (e.g., rotational movement, tilting movement, etc.) of the fixation members 34 to maintain alignment of the fixation members 34 along the shaft assembly 42, but other suitable configurations are contemplated.

In some example configurations, a distal end of the lumen 70 may include a tapered portion, where the tapered portion may be configured to facilitate maintaining the distal-most fixation member 34 within the shaft assembly 42 until the distal-most fixation member 34 is to be deployed. When the distal end of the lumen 70 is tapered and the distal-most fixation member 34 is to be deployed, the engagement member 78 of the first elongate member 56a may apply a force in the distal direction on the distal-most fixation member 34 and the portion of the filler component 68 extending inward toward the longitudinal axis L to define the tapered portion of the lumen 70 may flex outward (e.g., may compress) in response to engagement with the fixation member 34 to allow the fixation member 34 to pass through the lumen 70 and out of the shaft assembly 42 for deployment.

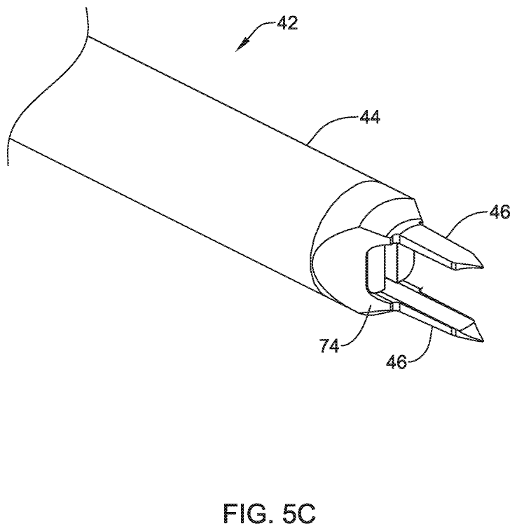

FIG. 8 schematically illustrates an exploded view of the fixation member actuation assembly 76. The first elongate member 56a and the second elongate member 56b of the actuation assembly 76 may be aligned relative to one another to facilitate maintaining a position of the fixation members 34 and incrementally advancing the fixation members 34 with the actuation cycle.

The first elongate member 56a may include one or more of the engagement members 78 (e.g., a plurality of first engagement members), each of which may be disposed and aligned along the first elongate member 56a. The first elongate member 56a may have at least an engagement member 78 for each fixation member 34 initially loaded into the delivery system 32. Similarly, the second elongate member 56b may include one or more of the engagement members 78 (e.g., a plurality of second engagement members), each of which are disposed and aligned along the second elongate member 56b. The second elongate member 56b may have at least an engagement member 78 for each fixation member 34 initially loaded into the delivery system 32. The fixation members 34 may be axially spaced apart from one another with each fixation member 34 engaged with one of the engagement members 78 of one or both of the first and second elongate members 56a, 56b.

FIG. 8 schematically illustrates the general alignment of the first engagement members 78 of the first elongate member 56a and the second engagement members 78 of the second elongate member 56b with each of the fixation members 34, respectively. For example, vertical dashed lines 84 depicted in FIG. 8 illustrate a vertical alignment of a distal end 86 of the first engagement members 78 of the first elongate member 56a with a proximal end 88 of the fixation member 34. Further, the second engagement members 78 of the second elongate member 56b may extend over the proximal end 88 of the fixation members 34. In other words, when assembled, the first engagement members 78 of the first elongate member 56a may be engaged with each of the fixation members 34 such that proximal-to-distal advancement of the first elongate member 56a may push each of the fixation members 34 in a distal direction along the second engagement members 78 of the second elongate member 56b.

The engagement members 78 may have any suitable configuration configured to engage the proximal end 88 of the fixation members 34 to advance the fixation members 34 distally and/or to prevent or mitigate proximal movement of the fixation members 34. For example, the distal end 86 of the engagement members 78 may have a surface configured to abut or mate with a surface of the proximal end 88 of the fixation member 34. In one example, the distal end 86 of the engagement member 78 may have a surface perpendicular to a direction of travel of the fixation members 34 that is configured to abut or mate with the proximal end 88 of the engagement member 78 also having a surface perpendicular the direction of travel of the fixation members 34.

The engagement members 78 may have any suitable configuration configured to flex in response to relative movement between the engagement members 78 and the fixation members 34. In one example, the engagement members 78 may include a first portion 78a extending distally from a base of the respective first elongate member 56a or second elongate member 56b, a second portion 78b extending distally from first portion 78a, and a third portion 78c extending distally from the second portion 78b. The first portion 78a of the engagement member 78 may be configured to flex relative to the base of the first or second elongate member 56a, 56b. The second portion 78b of the engagement member 78 may angle distally inward toward the fixation members 34 or a longitudinal axis of the outer shaft 44 or the shaft assembly 42. The third portion 78c of the engagement member 78 may be parallel or substantially parallel to the first portion 78a when the engagement member 78 is in a resting position or non-stressed position. Other suitable configurations of the engagement members 78 are contemplated.

The engagement members 78 may be formed in the first and/or the second elongate members 56a, 56b in any suitable manner. In one example, the engagement members 78 may be cut into the first and second elongate members 56a, 56b and bent to a desired shape that facilitates distally advancing the fixation members 34 and flexing the engagement members 78 outward relative to the fixation members 34 in response to contact with the fixation members 34 and/or contact with engagement member 78 of the opposing elongate member 56 when there is relative movement between the first and/or second elongate members 56a, 56b and the fixation member 34.

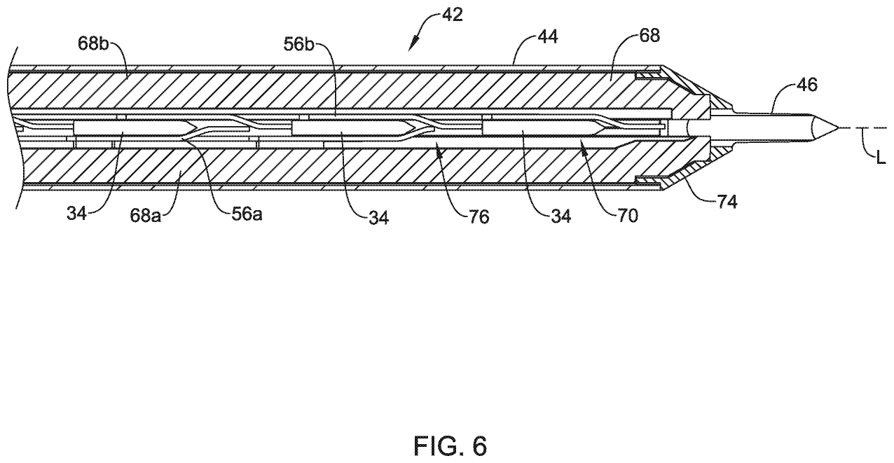

FIG. 9 schematically depicts a distal end of the fixation member actuation assembly 76, with the distal end 86 of the distal-most engagement member 78 of the first elongate member 56a engaged with the proximal end 88 of the distal-most fixation member 34. The distal-most engagement member 78 of the second elongate member 56b may be located distal of the distal-most fixation member 34 to prevent the fixation member 34 from inadvertently exiting the outer shaft 44. When the distal-most fixation member 34 is to be deployed, the first elongate member 56a may be advanced distally to move the distal-most fixation member 34 distally over the distal-most engagement member 78 of the second elongate member 56b.

Although other configurations are contemplated, the distal end 86 of the distal-most engagement member 78 of the first elongate member 56a may be configured to engage the proximal end 88 of the fixation member 34 and mate with the distal most engagement member 78 of the second elongate member 56b. As depicted in FIG. 9, the distal end 86 of the distal-most engagement member 78 of the first elongate member 56a may include a recess or cutout 90 (e.g., defining a distal fork configuration) to receive the distal-most engagement member 78 of the second elongate member 56b as the fixation member 34 is deployed from the outer shaft 44. Further, the portions of the distal end 86 of the distal-most engagement member 78 to the sides of the recess or cutout 90 may be aligned with or proximate with arms of the fixation member 34 such that force from the engagement member 78 may be applied to the fixation member 34 at or proximate the arms driven into the implant 12 and tissue of the patient. Applying force to the fixation member 34 from the distal-most engagement member 78 at or proximate the arms of the fixation member 34 may result in applying the force to a structurally strong portion of the proximal end 88 of the fixation member 34 and may facilitate advancing the fixation member through tissue, while mitigating damage to the fixation member 34. Other suitable configurations of the distal-most engagement members 78 of the first and/or second elongate members 56a, 56b are contemplated.

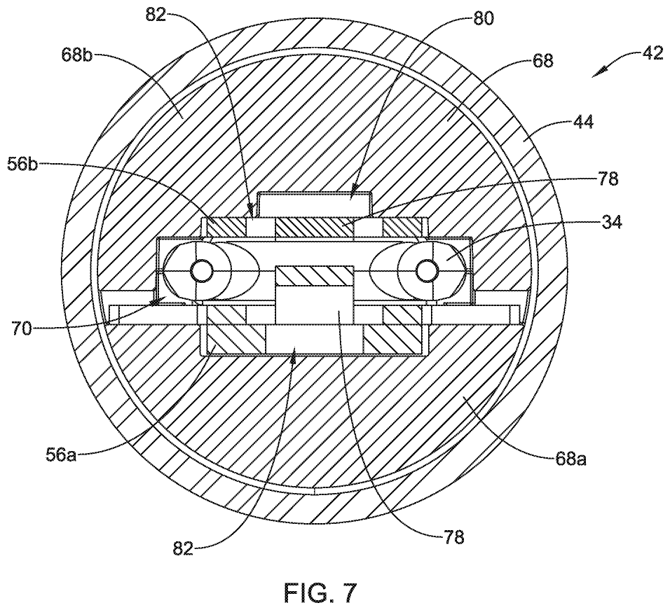

FIG. 10 schematically depicts a perspective view of the fixation member actuation assembly 76 with the first elongate member 56a coupled with the first filler component 68a and the second elongate member 56b extending along the first elongate member 56a. The first elongate member 56a may include one or more laterally extending slide wings 92 configured to facilitate and/or limit longitudinal adjustment of the first elongate member 56a relative to the first filler component 68a. For example, the first elongate member 56a may include two laterally extending slide wings 92 at one or more (e.g., multiple) axial locations along the first elongate member 56a, but other suitable configurations are contemplated. In some examples, the slide wings 92 may be aligned with and/or received in longitudinally extending channels or slots 94 of the first filler component 68a to facilitate and limit proximal and distal movement of the first elongate member 56a. When the second filler component 68b is positioned along the first filler component 68a, the second filler component 68b may cover or otherwise act as a cover for the slots 94 to secure the slide wings 92 within the slots 94. The first elongate member 56a may include one or more additional or alternative longitudinal adjustment facilitating/limiting components, as desired.

The second elongate member 56b may include one or more fixation wings 96. For example, the second elongate member 56b may include two laterally extending fixation wings 96 at multiple axial locations along the second elongate member 56b, but other suitable configurations are contemplated. The fixation wings 96 may be configured to engage the second filler component 68b (not shown in FIG. 10) to fix the second elongate member 56b relative to the second filler component 68b. The second elongate member 56b may include one or more additional or alternative fixation components, as desired.

In some configurations, the first filler component 68a may include one or more mating components 98. Example suitable mating components include, but are not limited to, holes, protrusions, pegs, and/or other suitable mating components. In one example and as depicted in FIG. 10, the first filler component 68a may include a plurality of mating components 98 configured as holes, but other suitable configurations of one or more similar or different mating components 98 are contemplated.

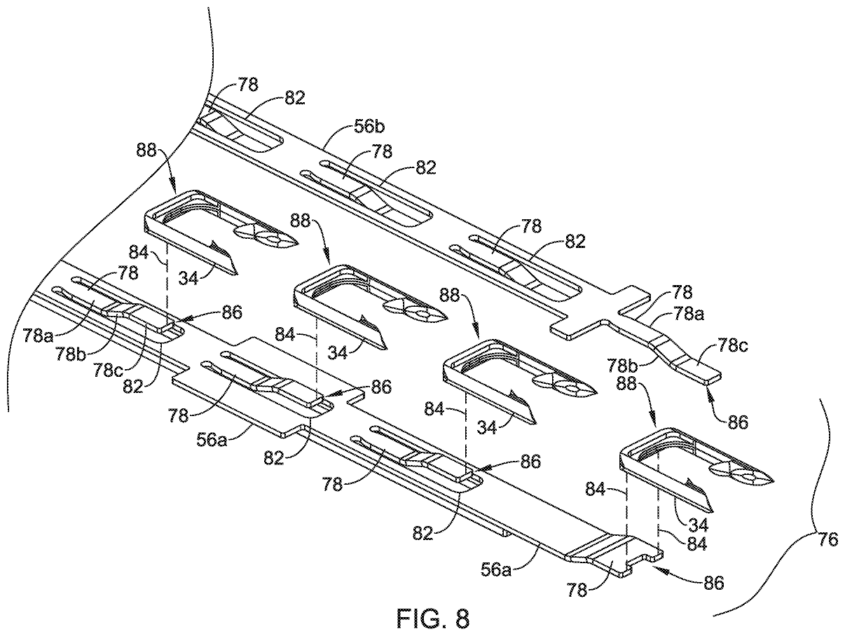

FIG. 11 schematically depicts a perspective view of the fixation member actuation assembly 76 with the second elongate member 56b coupled with the second filler component 68b and the first elongate member 56a extending along the second elongate member 56b. In some examples, the second elongate member 56b may include two laterally extending fixation wings 96 at multiple axial locations along the second elongate member 56b, but other suitable configurations are contemplated. In some examples, the fixation wings 96 may be aligned with and received in channels or slots 100 of the second filler component 68b to prevent or mitigate longitudinal movement of the second elongate member 56b relative to the second filler component 68b (e.g., to fix the second elongate member 56b relative to the second filler component 68b). When the first filler component 68a is positioned along the second filler component 68b, the first filler component 68a may cover or otherwise act as a cover for the slots 100 to secure the fixation wings 96 within the slots 100.

In some configurations, the second filler component 68b may include one or more mating components 98. In one example and as depicted in FIG. 10, the second filler component 68b may include a plurality of mating components 98 configured as pegs, but other suitable configurations of one or more similar or different mating components 98 are contemplated.

FIGS. 12-16 schematically illustrate a series of steps (e.g., an actuation or deployment cycle) which may be performed by the delivery system 32 to deploy a plurality of fixation members 34 in sequence. In FIGS. 12-16, the first housing member 48a has been removed to depict an interior of the handle 40 and the outer shaft 44 and the filler component 68 are in cross-section to depict an interior of the shaft assembly 42.

As an initial step, FIG. 12 illustrates the outer shaft 44 positioned adjacent to a target site within the body, with the distal tines 46 of the outer shaft 44 positioned adjacent to the implant 12. For simplicity, FIG. 12 schematically depicts the implant 12 positioned over a portion of the tendon 24. It can be appreciated, however, that the outer shaft 44 may be utilized to deploy the fixation members 34 in a variety of locations such as along the perimeter, the distal end, the proximal end and/or anywhere along the implant 12.

The actuation assembly 76, as depicted in FIG. 12, may be positioned in a resting or ready state. Further, the fixation member actuation assembly 76 may be positioned such that the distal-most fixation member 34 is proximal to the tines 46 of the outer shaft 44. As discussed above, the other fixation members 34 may remain aligned with, and spaced apart from, one another along the longitudinal axis L of the shaft assembly 42 and/or the outer shaft 44. With the actuation assembly 76 in the ready state, the handle 40 of the delivery system 32 may be in a relaxed or unactuated position, as depicted in FIG. 12.

A detailed view in FIG. 12 depicts the outer shaft 44 and the filler component 68 in a cross-section and the first elongate member 56a, the second elongate member 56b, and the fixation members 34 in a side view. The detailed view schematically illustrates the engagement member 78 of the second elongate member 56b in engagement with the fixation member 34 and the distal end 86 of the engagement member 78 of the first elongate member 56a ready to engage the proximal end 88 of the fixation member 34. The delivery system 32 may include additional fixation members 34 similarly arranged relative to other engagement members 78, as desired. The detailed view of FIG. 12 further illustrates the engagement members 78 extending away from (e.g., inward from) a base 79 of the first elongate member 56a and a base 79 of the second elongate member 56b, respectively, whereby the distal end regions 86 of the engagement members 78 may engage the proximal end 88 of one of the fixation members 34. It is noted that in other configurations, the delivery system 32 may only include one of the first and second elongate members 56a, 56b.

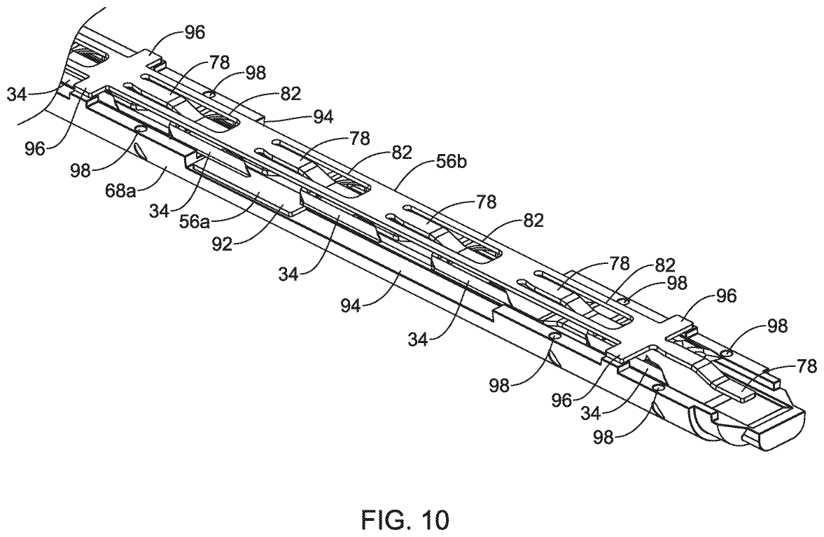

FIG. 13 schematically illustrates another step in the sequential deployment of the fixation members 34, with a detailed view similar to or the same as depicted in FIG. 12. As depicted in FIG. 13, the clinician has manipulated the handle 40 of the delivery system 32 distally to drive the tines 46 of the outer shaft 44 through the implant 12 and into the tendon 24, such that the outer shaft 44 has been driven forward to a position in which the distal face 74 of the outer shaft 44 may abut the implant 12. It can be appreciated that from this position, the distal-most fixation member 34 of the fixation member actuation assembly 76 may be advanced distally through the aperture created by the tines 46, whereby the fixation member 34 may be deployed directly into the tendon 24.

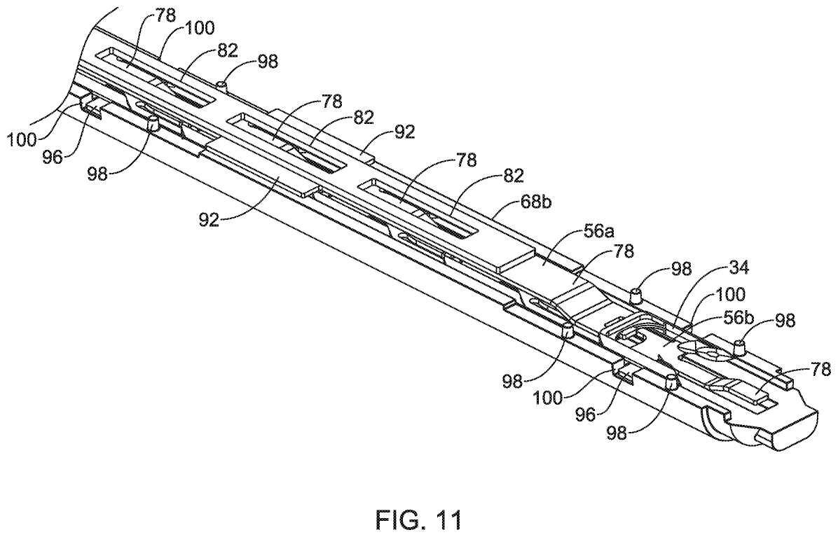

FIG. 14 schematically illustrates another step in the sequential deployment of the fixation members 34. As depicted in FIG. 14, the clinician has manipulated the actuator 50 of the handle 40 (e.g., a trigger of the handle assembly) of the delivery system 32 to drive the distal-most (e.g., leading) fixation member 34 into the tendon 24. As discussed herein, the handle 40 of the delivery system 32, through a variety of components, may manipulate the elongate shaft 54 and the first elongate member 56a distally relative to the second elongate member 56b to distally advance each of the fixation members 34 within the lumen 70 of the filler component 68 (e.g., within the elongate shaft 54). For example, as a clinician actuates the actuator 50 (e.g., squeezes a trigger or other suitable actuation mechanism) of the handle 40 of the delivery system 32, the linkage assembly 52 adjusts to advance the elongate shaft 54 against a bias of the spring 60 to advance the first elongate member 56a in a proximal-to-distal direction relative to the outer shaft 44, thereby engaging the distal end 86 of the engagement member 78 on the first elongate member 56a with the proximal end 88 of a proximate fixation member 34 and moving each of the fixation members 34 distally a corresponding amount. In other words, actuation of the actuator 50 of the handle 40 may cause the first elongate member 56a to move longitudinally in a distal direction relative to the outer shaft 44 to a distal position, simultaneously moving each of the fixation members 34 distally a corresponding amount. The proximal-to-distal advancement may deploy the distal-most fixation member 34 into the target site as well as advance each of the other fixation members 34 in the proximal-to-distal direction over (or under) the engagement members 78 of the second elongate member 56b (e.g., causing the engagement members 78 of the second elongate member 56b to flex laterally outward) to a next successive position within the outer shaft 44.

A detailed view of FIG. 14 depicts the outer shaft 44 and the filler component 68 in a cross-section and the first elongate member 56a, the second elongate member 56b, and the fixation members 34 in a side view. The detailed view schematically illustrates the engagement of the engagement members 78 of the first and second elongate members 56a, 56b with the fixation member 34. As depicted in FIG. 14, the distal end 86 of the engagement member 78 of the first elongate member 56a may engage the proximal end 88 of the fixation member 34 and advance distally to move the fixation member 34 distally relative to the second elongate member 56b, which remains fixed. As the fixation member 34 is moved distally, the fixation member 34 may engage the second portion 78b (e.g., a proximal surface or inward facing surface of the second portion 78b) of the engagement member 78 of the second elongate member 56b, which may result in the engagement member 78 flexing into the recess 80 of the second filler component 68b.

Additionally, it can be appreciated that as the clinician actuates the actuator 50 of or at the handle 40 to drive the fixation member 34 through the implant 12 and into the tendon tissue 24, the outer shaft 44 may, in some instances, retract proximally such that the tip of the tines 46 may retract from the implant 12. Because each of the fixation members 34 may be uniformly spaced from one another, the position of each of the fixation member 34 may be fixed relative to the first elongate member 56a and advanced relative to the second elongate member 56b as the fixation members 34 are distally advanced in unison.

With the actuator 50 fully actuated, a proximal end of the actuator 50 may engage the stop component 62 of or coupled with the housing 48. Further, the clinician may receive audible and/or tactile feedback in response to actuation of the actuator 50 as, before, or after the protrusion 66 engages the tactile feedback component 64.

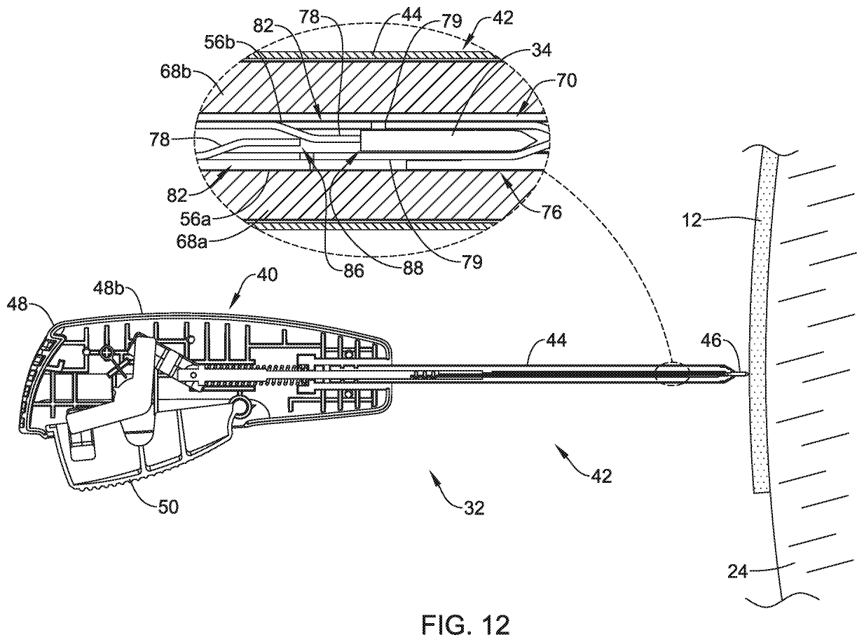

FIG. 15 schematically illustrates another step in the sequential deployment of the fixation members 34. Once the distal-most fixation member 34 has been deployed, the fixation member actuation assembly 76 may be reset to a resting or ready state to prepare the fixation member actuation assembly 76 to advance (e.g., “cycle”) another fixation member 34 out of the outer shaft 44. For example, as the clinician releases the actuator 50 on or at the handle 40, the first elongate member 56a may be advanced proximally (e.g., retracted, as shown by the arrow 102) relative to the second elongate member 56b. In some examples, the first elongate member 56a may be automatically advanced proximally once the clinician releases the actuator 50 due to the spring 60 acting on the elongate shaft 54 and the linkage assembly 52, which in turn, pulls the first elongate member 56a proximally. The second elongate member 56b may remain stationary as the first elongate member 56a is advanced proximally or withdrawn. In some examples, the engagement members 78 of the second elongate member 56b may prevent or limit proximal movement of the fixation members 34 as the first elongate member 56a is moved in the proximal direction and is reset.

A detailed view of FIG. 15 depicts the outer shaft 44 and the filler component 68 in a cross-section and the first elongate member 56a, the second elongate member 56b, and the fixation members 34 in a side view. The detailed view schematically illustrates the engagement of the engagement members 78 of the first and second elongate members 56a, 56b with the fixation member 34. As depicted in FIG. 15, the first elongate member 56a is advanced proximally relative to the second elongate member 56b, which remains fixed, and the distal end 86 of the engagement member 78 of the second elongate member 56b may engage the proximal end 88 of the fixation member 34 to prevent or limit proximal movement of the fixation member 34. As the first elongate member 56a is moved proximally, the second portion 78b (e.g., a proximal surface or inward facing surface of the second portion 78b) of the engagement member 78 of the first elongate member 56a may engage the fixation member 34 prevented from moving in the proximal direction by the engagement member 78 of the second elongate member 56b, which may cause the engagement member 78 of the first elongate member 56a to flex into the opening 82 in the base of the first elongate member 56a and/or into one or more other suitable openings or recesses.

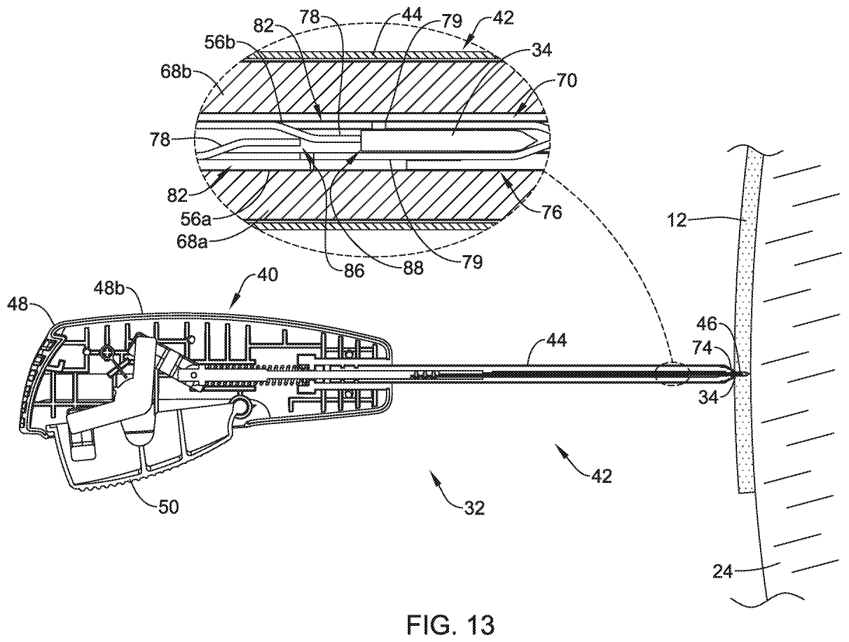

FIG. 16 schematically illustrates another step in the sequential deployment of the fixation members 34. For example, FIG. 16 depicts the actuator 50 in a fully relaxed position with the first elongate member 56a advanced proximally in a fully retracted position relative to the second elongate member 56b, which remains longitudinally stationary relative to the elongate shaft 54 and/or the handle 40 and engaged with the fixation member 34, as depicted in the detailed view of FIG. 16. The detailed schematically depicts the outer shaft 44 and the filler component 68 in a cross-section and the first elongate member 56a, the second elongate member 56b, and the fixation members 34 in a side view

After the first elongate member 56a is retracted back to a proximal position and the distal ends 86 of the engagement members 78 of the second elongate member 56b is engaging the proximal end of the fixation members 34, the fixation member actuation assembly 76 has completed one “cycle,” whereby the distal-most fixation member 34 was deployed into the tendon 24 and the remaining fixation members 34 have moved distally one position in the outer shaft 44 to ready deployment of the next distal-most fixation member 34. After a distal-most fixation member 34 is deployed and the actuation mechanism reset, the outer shaft 44 may be repositioned, whereby the next fixation member 34 now becomes the distal-most fixation member 34 and may be deployed in the same sequence of steps described above with respect to FIGS. 12-16 and/or with other suitable steps. The cycle may continue with a clinician repositioning the delivery system 32 and deploying the fixation members 34 until all the fixation members 34 are implanted, without having to remove the delivery system 32 from an implant site.

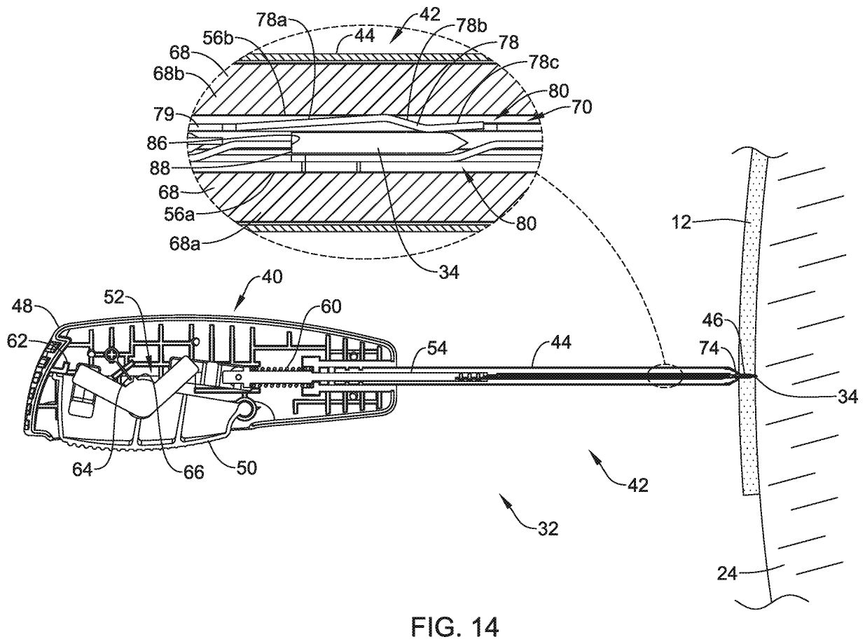

FIG. 17 schematically illustrates a cross-section of inner components of an illustrative configuration of the delivery system 32, with the first housing member 48a removed and depicting a second housing member 48b of the handle 40. Similar to as discussed with respect to the illustrative configuration of the delivery system 32 depicted in FIG. 4, the illustrative configuration of the delivery system 32 depicted in FIG. 17 may include the actuator 50 coupled with the linkage assembly 52. The linkage assembly 52 may be further coupled to an elongate shaft 54 (e.g., push rod and/or other suitable elongate shaft) with the spring 60 biasing the actuator 50, the linkage assembly 52, and the elongate shaft 54 to a resting, unactuated position. As depicted in FIG. 17, the actuator 50, the linkage assembly 52, the coupling between the linkage assembly 52 and the elongate shaft 54, and the spring 60 may be configured similar to the actuator 50, the linkage assembly 52, the coupling between the linkage assembly 52 and the elongate shaft 54, and the spring 60 as discussed with respect to the configuration of the delivery system 32 depicted in FIG. 4.

The shaft assembly 42 may include the outer shaft 44 extending distally from the handle 40. In some examples, the outer shaft 44 may define a lumen 104 configured to receive fixation members 34 and/or one or more elongate members 56 including, but not limited to, an elongate member 56 movable along the outer shaft 44.

The outer shaft 44 may have any suitable cross-sectional shape configured to receive the elongate member(s) 56 and the fixation members 34 including, but not limited to, a circular cross-sectional shape, an oval cross-sectional shape, a rectangular cross-sectional shape, and/or other suitable cross-sectional shape. In one example, the outer shaft 44 may have an oval cross-sectional shape that entirely or at least partially defines the lumen 104.

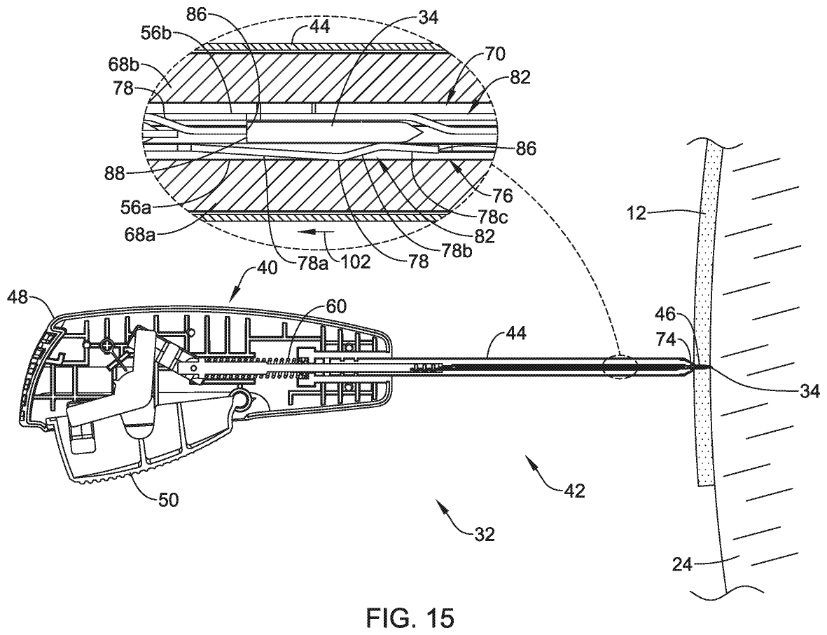

FIG. 18 schematically depicts a detailed view of distal end of the handle 40 and a proximal end of the shaft assembly 42 of the illustrative configuration of the delivery system 32 depicted in FIG. 17. The outer shaft 44 may be coupled with the housing 48 in any suitable manner including with, but not limited to, threaded connections, screws, adhesives, press-fit connections, friction fit connections, snap connections ultrasonic welding, over molding, a tab connection, and/or one or more other suitable types of coupling.

The first elongate member 56a may couple with the elongate shaft 54 and may be configured to move longitudinally (e.g., in a proximal direction and/or a distal direction) with the elongate shaft 54 as the actuator 50 is actuated and un-actuated (e.g., released). The first elongate member 56a may be coupled with the elongate shaft 54 in any suitable manner. For example, a proximal end portion of the first elongate member 56a may be coupled with a distal end portion of the elongate shaft 54 using one or more coupling techniques including, but not limited to, threaded connections, screws, adhesives, press-fit connections, friction fit connections, snap connections ultrasonic welding, over molding, a tab connection, and/or one or more other suitable types of coupling. In one example, the proximal end portion of the first elongate member 56a may be coupled with the distal end portion of the elongate shaft 54 via one or more screws, but other suitable configurations are contemplated.

The second elongate member 56b may engage or otherwise be coupled with the handle 40 (e.g., the housing 48 of the handle 40 and/or other suitable component of the handle 40), the outer shaft 44, and/or other component(s) of the delivery system 32 configured to be fixed relative to the handle 40. In some examples, the second elongate member 56b may be coupled with the housing 48 of the handle 40 such that the second elongate member 56b may remain fixed relative to the handle 40. When coupled with the housing 48, the second elongate member 56b may be coupled with the housing 48 in any suitable manner including, but not limited to, threaded connections, screws, adhesives, press-fit connections, friction fit connections, snap connections ultrasonic welding, over molding, tab connection, and/or one or more other suitable types of coupling. In one example, a proximal end portion of the second elongate member 56b may have a tab 106 that may be inserted into a slot 108 of the housing 48, where the tab 106 and the slot 108 may be transverse (e.g., perpendicular or otherwise transverse) to longitudinal movement of the elongate shaft 54 and the first elongate member 56a to prevent or mitigate longitudinal movement of the second elongate member 56b.

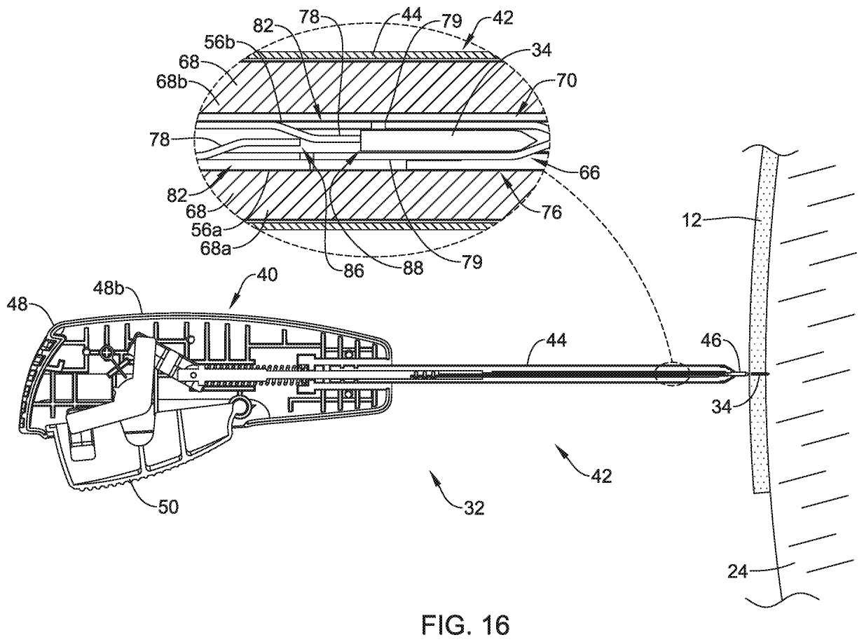

FIG. 19 schematically illustrates a cross-section of the shaft assembly 42 of the illustrative configuration of the delivery system 32 depicted in FIG. 17. FIG. 19 illustrates that the shaft assembly 42 may include the fixation member actuation assembly 76 positioned within the lumen 104 of the outer shaft 44. The fixation member actuation assembly 76 may include one or more components, which collectively, work together to deploy the fixation members 34 out of the distal end of the shaft assembly 42.

The fixation member actuation assembly 76 may include the first elongate member 56a and the second elongate member 56b. The first elongate member 56a and the second elongate member 56b may extend through the lumen 104 of the outer shaft 44.

The first elongate member 56a may be configured to longitudinally adjust relative to the outer shaft 44 and the second elongate member 56b to advance the fixation members 34 distally and deploy a distal-most fixation member 34 through the tines 46. The second elongate member 56b may be longitudinally fixed relative to the outer shaft 44 to prevent proximal movement of the fixation members 34 as the first elongate member 56a is adjusted in the proximal direction during the actuation cycle.

As depicted in FIG. 19, a plurality of fixation members 34 may be oriented in longitudinal alignment with the longitudinal axis L of the shaft assembly 42 or the outer shaft 44 with distal points of the fixation members 34 pointed toward the distal end of the outer shaft 44. As shown in FIG. 19 and similar to as discussed with respect to FIG. 6, the fixation members 34 may be spaced away from one another along the longitudinal axis L such that adjacent fixation members 34 do not directly contact one another. Further, the fixation members 34 may be positioned along the longitudinal axis L between the first elongate member 56a and the second elongate member 56b. As such, the delivery system 32 may be initially loaded with a plurality of fixation members 34, such as four or more fixation members 34, six or more fixation members 34, or eight or more fixation members 34 for sequential deployment from the delivery system 32, which may be sequentially advanced out of the distal end of the outer shaft 44 as the fixation member actuation assembly 76 is manipulated via the handle 40 (not shown in FIG. 19).

The engagement members 78 may be configured to flex. For example, as the engagement members 78 of the second elongate member 56b engage the fixation members 34 and/or the engagement members 78 of the first elongate member 56a when the fixation members 34 advance distally along the shaft assembly 42, the engagement members 78 of the second elongate member 56b may flex laterally outward from the longitudinal axis L. In another example, as the engagement members 78 of the first elongate member 56a engage the fixation members 34 and/or the engagement members 78 of the second elongate member 56b when the first elongate member 56a is adjusted proximally during the actuation cycle, the engagement members 78 of the first elongate member 56a may flex laterally outward from the longitudinal axis L.

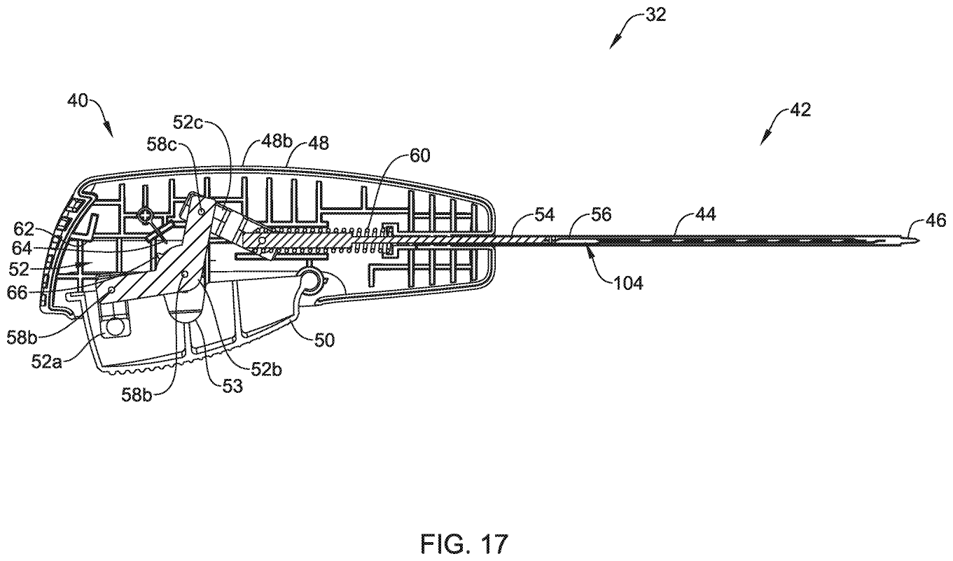

FIG. 20 depicts a cross-section taken at an axial location along the shaft assembly 42, which depicts the outer shaft 44, the first elongate member 56a, the second elongate member 56b, and the fixation member 34. As depicted in FIG. 20, the outer shaft 44 may have an oval cross-sectional shape, but other suitable configurations are contemplated.

The lumen 104 of the outer shaft 44 may at least partially define space for the engagement members 78 to flex laterally outward from the longitudinal axis L. For example, due to an oval shape or other suitable shape of the outer shaft 44, there may be space in the lumen 104 between the first elongate member 56a and an inner surface of the outer shaft 44 and a space in the lumen 104 between the second elongate member 56b and the inner surface of the outer shaft 44 for the engagement members 78 to flex laterally outward from the longitudinal axis L as the engagement members 78 engage the fixation members 34 and/or engagement members 78 of the opposing elongate member 56. Other suitable configurations for facilitating flexing of the engagement members 78 are contemplated.

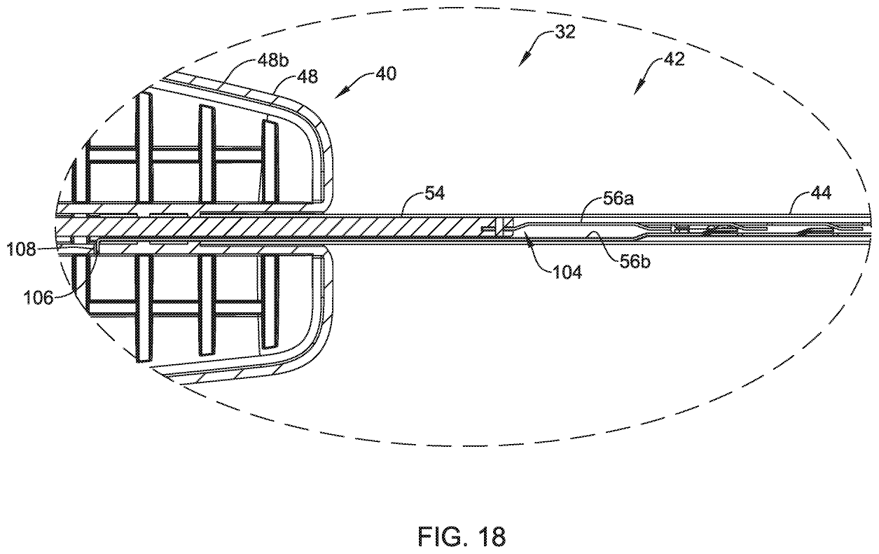

FIG. 21 schematically depicts a side view of the fixation member actuation assembly 76, which may include the first elongate member 56a and the second elongate member 56b. In some examples and as depicted in FIG. 21, the engagement members 78 of each of the first elongate member 56a and the second elongate member 56b may extend inward from a base of the respective first and second elongate members 56a, 56b. In some examples, the engagement members 78 of the first and second elongate members 56a, 56b may extend inward so as to be located within a same plane extending longitudinally along the first elongate member 56a and the second elongate member 56b (e.g., the engagement members 78 may be aligned along the longitudinal axis L, not shown in FIG. 21). Other suitable configurations of the engagement members 78 of the first elongate member 56a and the second elongate member 56b are contemplated.

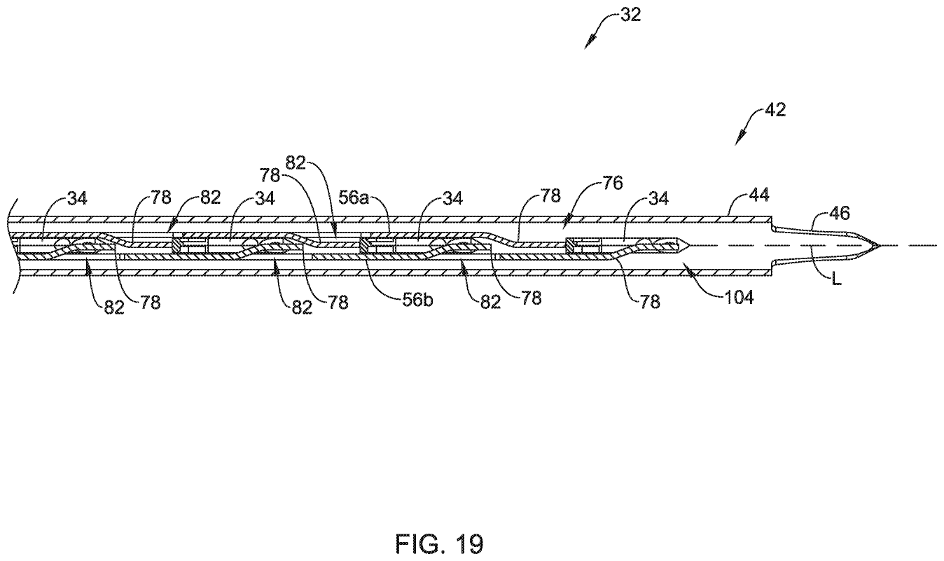

FIG. 22 schematically depicts a distal end of the fixation member actuation assembly 76 of the illustrative configuration of the delivery system 32 depicted in FIG. 17, with the distal end 86 of the distal-most engagement member 78 of the first elongate member 56a proximate the proximal end 88 of the distal-most fixation member 34. The distal-most engagement member 78 of the second elongate member 56b may be located distal of the distal-most fixation member 34 to prevent the fixation member 34 from inadvertently exiting the outer shaft 44. When the distal-most fixation member 34 is to be deployed, the first elongate member 56a may be advanced distally to move the distal-most fixation member 34 distally over the distal-most engagement member 78 of the second elongate member 56b and out of the outer shaft 44 (not depicted in FIG. 22).

Although other configurations are contemplated, the distal end 86 of the distal-most engagement member 78 of the first elongate member 56a may be configured to engage the proximal end 88 of the fixation member 34 and mate with the engagement members 78 of the second elongate member 56b. As depicted in FIG. 22, the distal end 86 of the distal-most engagement member 78 of the first elongate member 56a may include the recess or cutout 90 (e.g., defining a distal fork configuration) to receive the engagement members 78 of the second elongate member 56b as the fixation member 34 is deployed from the outer shaft 44. Further, the portions of the distal end 86 of the distal-most engagement member 78 to the sides of the recess or cutout 90 may be aligned with or proximate with arms of the fixation member 34 such that force from the engagement member 78 may be applied to the fixation member 34 at or proximate the arms driven into the implant 12 and tissue of the patient. Applying force to the fixation member 34 from the distal-most engagement member 78 at or proximate the arms of the fixation member 34 may result in applying the force to a structurally strong portion of the proximal end 88 of the fixation member 34 and may facilitate advancing the fixation member through tissue, while mitigating damage to the fixation member 34. Other suitable configurations of the distal-most engagement members 78 of the first and/or second elongate members 56a, 56b are contemplated.

The engagement members 78 of the illustrative configuration of the delivery system 32 depicted in FIG. 17 may have any suitable configuration configured to flex in response to relative movement of the engagement members 78 and/or the fixation members 34. In one example, the engagement members 78 may include a first portion 78a extending distally from a base 79 of the respective first elongate member 56a or the second elongate member 56b, a second portion 78b extending distally from first portion 78a, and a third portion 78c extending distally from the second portion. The first portion 78a of the engagement member 78 may be configured to flex relative to a base of the first and/or second elongate member 56a, 56b. The second portion 78b of the engagement member 78 may angle distally inward toward the fixation members 34 or a longitudinal axis of the shaft assembly 42 or the outer shaft 44. The third portion 78c of the engagement member 78 may be parallel or substantially parallel to the first portion 78a when the engagement member 78 is in a resting position or a non-stressed position. Other suitable configurations of the engagement members 78 are contemplated.

The engagement members 78 of the illustrative configuration of the delivery system 32 depicted in FIG. 17 may be formed in the first and/or the second elongate members 56a, 56b in any suitable manner. In one example, the engagement members 78 may be cut into the first and second elongate members 56a, 56b and bent to a desired shape (e.g., the shape depicted in FIG. 22 and/or other suitable shape) that facilitates distally advancing the fixation members 34 and flexing the engagement members 78 outward relative to the fixation members 34 in response to contact with the fixation members 34 when there is relative movement between the first and/or second elongate members 56a, 56b and the fixation member 34. The first elongate member 56a and/or the second elongate member 56b may have an opening 82 for receiving a flexing engagement member 78. In one example, the engagement members 78 may be configured to flex into and/or through the opening 82 to the space between the first and/or the second elongate members 56a, 56b and an inner surface of the outer shaft 44 in response to the second portion 78b and/or other suitable portion of the engagement member 78 contacting the fixation member 34 and/or the other elongate member 56 from which the flexing engagement member 78 does not extend.

The illustrative configuration of the delivery system 32 depicted in FIG. 17 may operate in any suitable manner to deploy the fixation members 34 at one or more target sites. For example, the illustrative configuration of the delivery system 32 depicted in FIG. 17 may operate in a manner similar to how the configuration of the delivery system 32 depicted in FIG. 4 operates, as depicted in and discussed with respect to FIGS. 12-16.

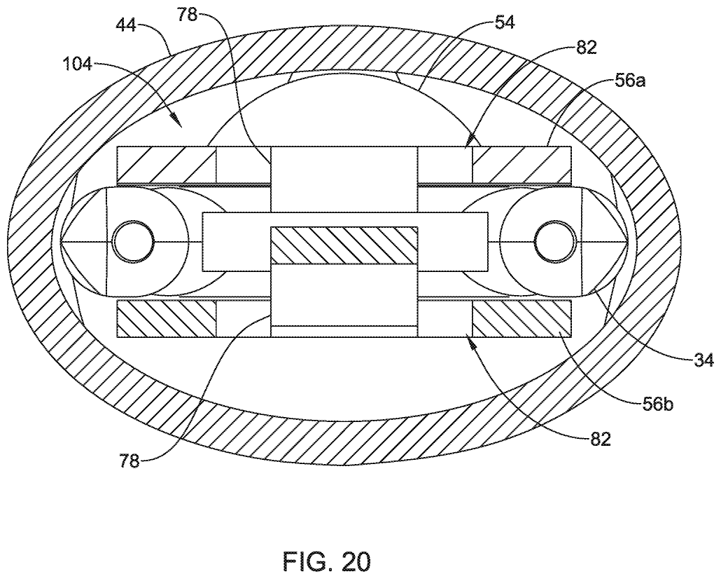

FIG. 23 is a schematic perspective view of an illustrative configuration of a fixation member 34. Although the various parts of the fixation member 34 are depicted in relative proportion to other parts of fixation member 34, other configurations in size and orientation of the various parts are also contemplated in other examples.

In some examples, the fixation member 34 may be in the form of a staple or anchor including a first arm 110a, second arm 110b, and a bridge 112 extending between the first and second arms 110a/110b, where the fixation member 34 has a proximal end 88 and a distal end 114. The bridge 112 may be located at or proximate the proximal end 88 of the fixation member 34 and may abut or extend from or be adjacent to, the proximal end of the first arm 110a and the proximal end of the second arm 110b, as depicted in FIG. 23 for example. In some examples, the first arm 110a may include a first anchor portion 116a and the second arm 110b may include a second anchor portion 116b. In some examples, the anchor portions 116a/116b may each include a projection 118 (e.g., an interior prong). The first projections 118 on each of the first anchor portion 116a and the second anchor portion 116b may extend in the proximal direction and inward toward an arm from which the anchor portion does not extend. Having the projection 118 on each of the first anchor portion 116a and the second anchor portion 116b in the proximal direction and inward may permit the fixation member 34 to slide and/or advance through the shaft assembly 42, use inner surface of the tines 46 as tracks, and to engage with tissue, such as tendon tissue, as and/or after the fixation member 34 is deployed.

FIG. 23 depicts the bridge 112 of the fixation member 34 may include a surface 120 (e.g., a planar surface) extending perpendicular to or substantially perpendicular to a longitudinal axis of the fixation member 34 (e.g., perpendicular to a longitudinal axis along which the fixation member 34 advances in the distal direction). Other suitable configurations of the surface 120 are contemplated. The surface 120 may be configured to engage the distal end 86 of the engagement members 78. Utilizing the surface 120 depicted in FIG. 23 and moving the projections 118 on an inner side of the arms 110a, 110b may result in a stiff fixation member 34 that mitigates tilting or rotational movement of the fixation member 34 as the fixation member 34 is advanced along the shaft assembly 42 inserted into the implant 12 and tissue.

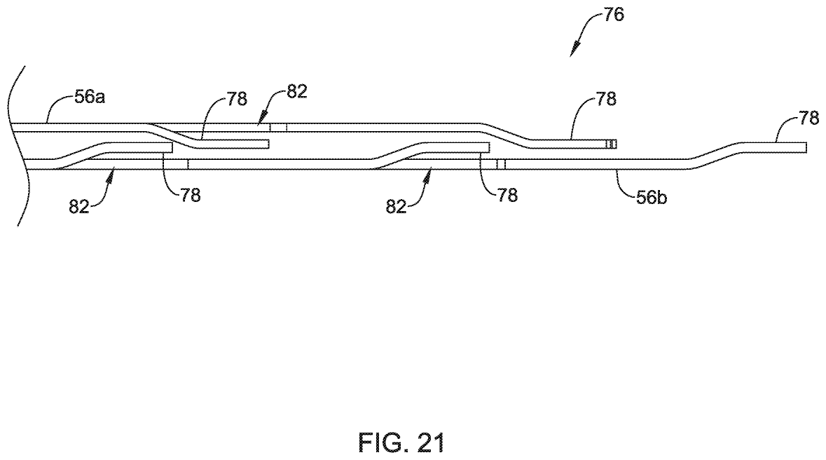

FIG. 24 schematically depicts a side view of the fixation member 34 depicted in FIG. 23. As discussed, the anchor portions 116a/116b may each include the projection 118. Further, FIG. 24 illustrates that a distal end of each of the anchor portions 116a/116b may include a sharp and/or pointed end 122. The pointed ends 122 may aid the fixation member 34 in piercing through the implant 12 and into tissue, such as tendon tissue, upon deployment of the fixation member 34.

As depicted FIG. 24, the bridge 112 may include one or more reinforcing components 124. For example, the one or more reinforcing components 124 may extend between one or both arms 110a, 110b and the bridge 112 to support the bridge 112 as force is applied to the fixation member 34 during deployment and insertion into the implant and/or tissue.

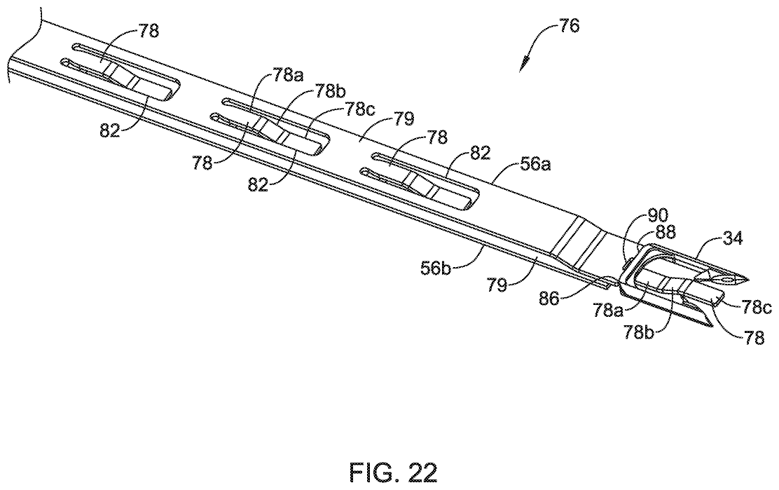

FIG. 25 schematically depicts a distal perspective view of the illustrative configuration of the fixation member 34 depicted in FIG. 23. In some examples, the first arm 110a and/or the second arm 110b may include one or more openings or holes 126 (e.g., apertures). The openings or holes 126 may extend into the arms 110a, 110b and/or the anchor portions 116a, 116b of the arms 110a, 110b. The openings or holes 126 may facilitate saving material when forming the fixation member 34 and/or inserting the fixation member 34 into an implant and/or tissue. In some examples and although not depicted, the openings or holes 126 may extend through the arms 110a, 110b and/or the anchor portions 116a, 116b such that one or more rails may extend through the arms 110a, 110b to guide the fixation members 34 during deployment.

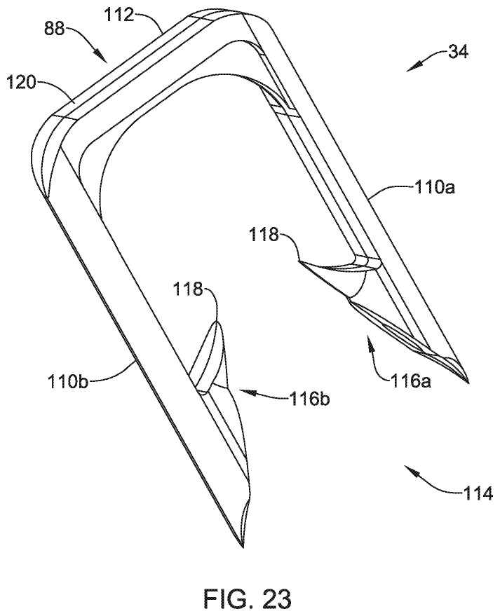

FIG. 26 schematically depicts an alternative shaft assembly 242 for the fixation member delivery system 32, described above. It is noted that the shaft assembly 242 may be attached to the handle 40, and its internal components described above, and extend distally from the handle 40. The shaft assembly 242 may include an outer shaft 244, similar to the outer shaft 44 described above. A pair of tines 46 (one of which is shown in FIG. 26) may be formed at and/or extend from the distal end of the outer shaft 244. As shown in FIG. 26, the pair of tines 46 may be formed integral with the outer shaft 244. However, in other instances, the pair of tines 46 may be included with a separate distal tip secured to the distal end of the outer shaft 244. Other configurations are also contemplated. A plurality of fixation members 34 may be loaded within the shaft assembly 242 for sequential deployment therefrom. The shaft assembly 242 may include a first elongate member 56a and a second elongate member 56b extending through the lumen 70 of the outer shaft 244. The first elongate member 56a and the second elongate member 56b may be configured to sequentially advance the fixation members 34 distally within the outer shaft 242 to sequentially deploy a distalmost one of the fixation members 34, as described above. For example, the second elongate member 58b may be held stationary within the lumen 70 of the outer shaft 244 as the first elongate member 58a is reciprocated axially to advance the fixation members 34 distally within the outer shaft 244. Further aspects of the shaft assembly 242 may be similar to the components of the shaft assembly 42 and their interaction with components of the handle 40, described above, and thus will not be repeated.

The shaft assembly 242 may include a first, upper insert 210 and/or a second, lower insert 220 positioned within the lumen 70 of the outer shaft 244 proximate the distal end of the outer shaft 244. The upper insert 210 and the lower insert 220 may be formed separate from the outer shaft 244 and secured to an interior of the outer shaft 244. For example, pins 230 may be provided to secure the upper insert 210 and the lower insert 220 to an interior of the outer shaft 244 proximate the distal end of the outer shaft 244. Other means of securement are also contemplated, such as, adhesive, heat stakes, welding, etc. The upper insert 210 and the lower insert 220 may be configured to facilitate deployment of the distalmost fixation member 34 from the distal end of the outer shaft 244.

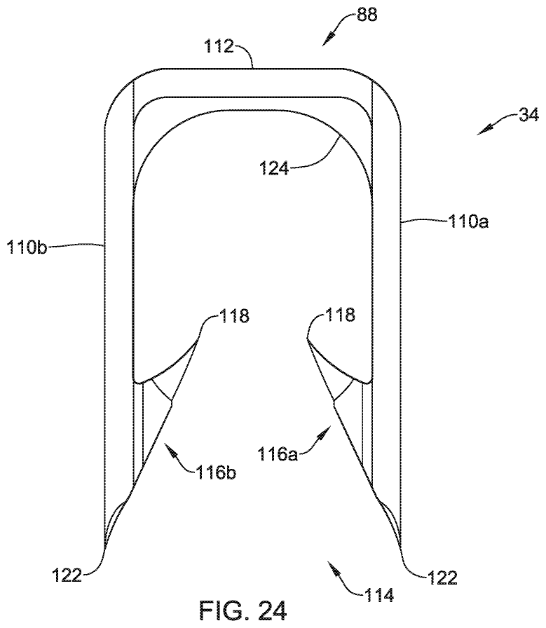

FIG. 27 is an exploded view of the components of the shaft assembly 242, including the upper insert 210 and the lower insert 220. The upper insert 210 may include a bore 232 for receiving each pin 230 attaching the upper insert 210 to the interior of the outer shaft 244. Similarly, the lower insert 220 may include a bore 232 for receiving each pin 230 attaching the lower insert 220 to the interior of outer shaft 244. Additionally, the upper insert 210 may include a notch 212 extending distally from a proximal end of the upper insert 210. The upper insert 210 may include a pair of tabs 214 extending along opposite sides of the notch 212. Furthermore, the lower insert 220 may include a notch 222 extending distally from a proximal end of the lower insert 220. The lower insert 220 may include a pair of tabs 224 extending along opposite sides of the notch 222.

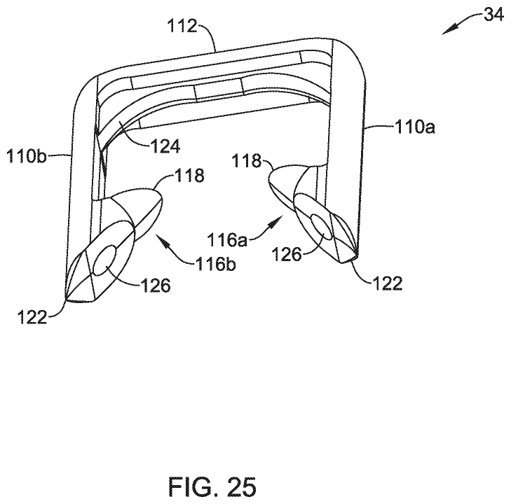

Turning to FIG. 28, the tabs 214 of the upper insert 210 may include a tapered surface 215 tapering distally toward the longitudinal axis of the shaft assembly 242. Likewise, the tabs 224 of the lower insert 220 may include a tapered surface 225 tapering distally toward the longitudinal axis of the shaft assembly 242. Thus, the tapered surface 215 may taper towards the tapered surface 225 in a distal direction and/or the tapered surface 225 may taper towards the tapered surface 225 in a distal direction. The tapered surfaces 215, 225 may help guide the fixation member 34 to be advance distally between the upper insert 210 and the lower insert 220 as the fixation member 34 is being deployed from the distal end of the shaft assembly 242. In some instances, the proximal end of the upper insert 210 may extend proximal of the proximal end of the lower insert 220, such that the fixation member 34 may engage or contact the tapered surface 215 prior to engaging or contacting the tapered surface 225. In other instances, the relative staggering of the tapered surfaces 215, 225 may be reversed, with the lower insert 220 extending proximal of the proximal end of the upper insert 210. The upper insert 210 may be spaced apart from the lower insert 220 a distance marginally greater than a thickness of the fixation member 34 to permit the fixation member 34 to pass between the upper insert 210 and the lower insert 220, while restraining the orientation of the fixation member 34 as the fixation member 34 is advanced distally between the upper insert 210 and the lower insert 220.

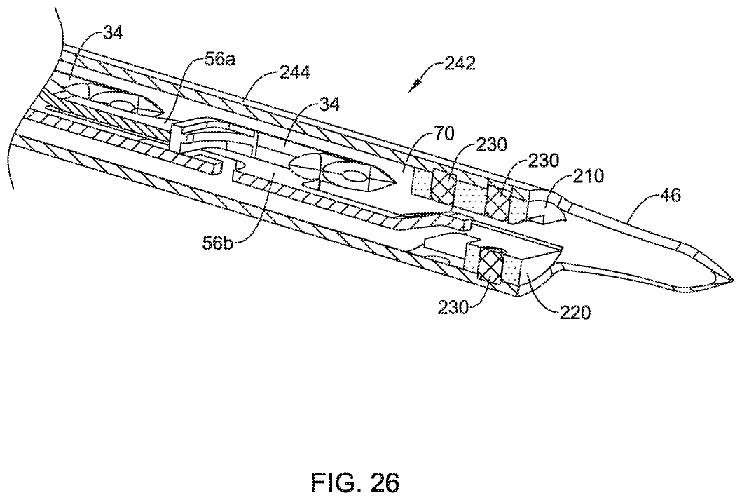

FIG. 29 further illustrates the interaction of the second elongate member 56b with the lower insert 220. Namely, as shown in FIG. 29, the distalmost engagement member 78 of the second elongate member 56b may extend distal of the proximal end of the lower insert 220. The engagement member 78 may extend into the notch 222 of the lower insert 220 with distal end 86 of the engagement member 78 spaced proximal of a distal extent of the notch 222 of the lower insert 220. For example, at least the third portion 78c that extends to the distal end 86 of the engagement member 78 may be positioned within the notch 222 of the lower insert 220. The engagement member 78, or at least the portion that extends into the notch 222, may have a width W₁, while the notch 222 may have a width W₂ greater than the width W₁ of the engagement member 78 to provide clearance between the engagement member 78 and the tabs 224 arranged on either side of the engagement member 78. Thus, the engagement member 78 may be deflected into the notch 222 as the fixation member 34 engages and passes by the third portion 78c of the engagement member 78.

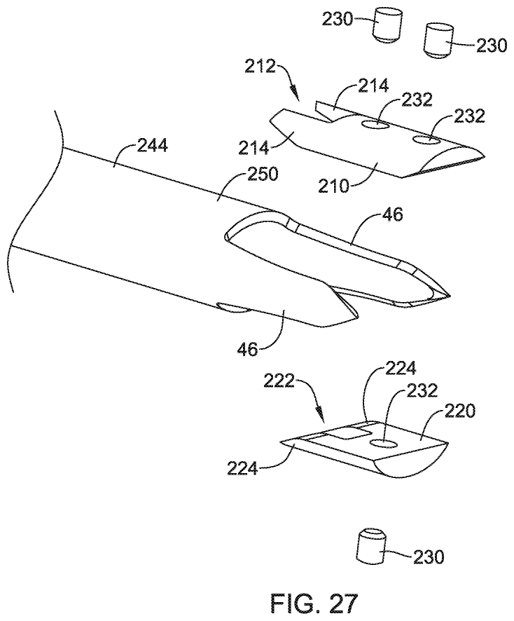

FIG. 30 further illustrates the interaction of the first elongate member 56a with the upper insert 210. Namely, as shown in FIG. 30, the distalmost engagement member 78 of the first elongate member 56a may extend distal of the proximal end of the upper insert 210 when actuated to a distal extent of the longitudinal movement of the first elongate member 56a. A portion of the engagement member 78 may extend into the notch 212 of the upper insert 210 as the engagement member 78 advances distally to deploy the fixation member 34. For example, at least a mid-region of the engagement member 78 may be positioned within the notch 212 of the upper insert 210 when actuated to a distal extent of the longitudinal movement of the first elongate member 56a. The engagement member 78, or at least the portion that extends into the notch 212, may have a width W₃, while the notch 212 may have a width W₄ greater than the width W₃ of the engagement member 78 to provide clearance between the engagement member 78 and the tabs 214 arranged on either side of the engagement member 78. Thus, the engagement member 78 may be advanced into the notch 212 as the engagement member 78 advances the fixation member 34 distally of the upper insert 210.

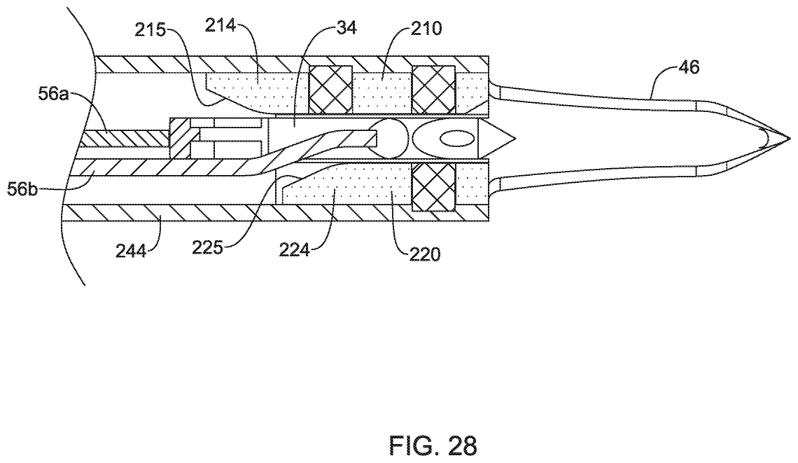

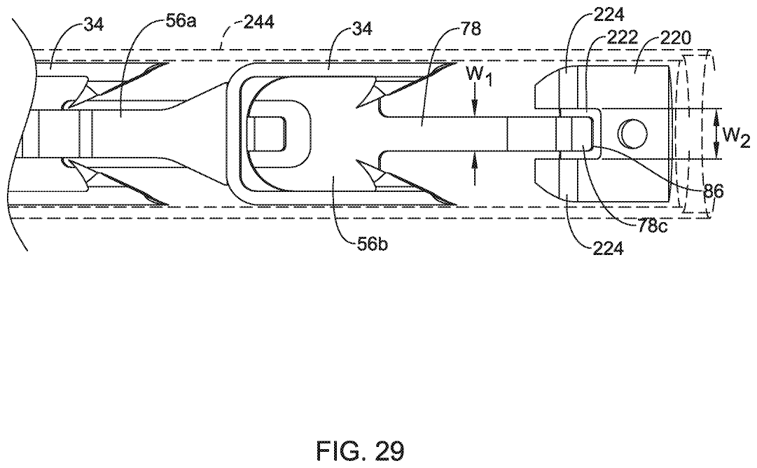

FIGS. 31A-31C illustrate a variation in which the upper and lower inserts, described above, are integrally formed with a distal tip 350, which may be formed separately and attached to the distal end of an outer shaft 344 (see FIG. 32). In other words, in the embodiment of FIGS. 31A-31C, the upper tabs 314a, the lower tabs 314b, and the body 351 (as well as the tines 46) are monolithically formed as a single piece with a distal tip 350. The distal tip 350 may be secured to the distal end of the outer shaft 344 (shown in FIG. 32).

The upper tabs 314a may include a tapered surface 315 tapering distally toward the longitudinal axis of the shaft assembly 242. Likewise, the lower tabs 314b may include a tapered surface 315 tapering distally toward the longitudinal axis of the shaft assembly 242. Thus, the tapered surface 315 of the upper tabs 314a may taper towards the tapered surface 315 of the lower tabs 314b in a distal direction and/or the tapered surface 315 of the lower tabs 314b may taper towards the tapered surface 315 of the upper tabs 314a in a distal direction. For example, as shown in FIG. 32, the tapered surfaces 315 may be a first distance apart H₁ at a proximal end of the tabs 314a, 314b, and the tapered surfaces 315 may be a second distance apart H₂ at a distal end of the tabs 314a, 314b, in which the second distance H₂ is less than the first distance H₁. The tapered surfaces 315 may help guide the fixation member 34 to be advance distally through the interior passage 318 of the distal tip 350 as the fixation member 34 is being deployed from the distal end of the shaft assembly 242.

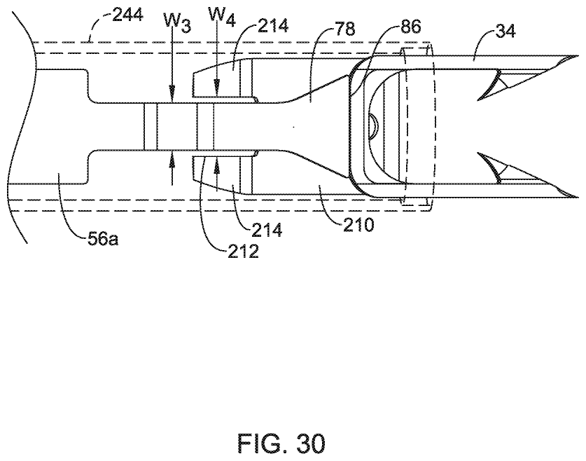

The interior passage 318 may have a corresponding height H₂ marginally greater than a thickness of the fixation member 34 to permit the fixation member 34 to pass through the interior passage 318, while restraining the orientation of the fixation member 34 as the fixation member 34 is advanced distally through the interior passage 318 of the distal tip 350. As shown in FIG. 31C and FIG. 33, the distal tip 350 may define a channel 317 between the upper tabs 314a and the lower tabs 314b. The channel 317, which may accommodate the profile of a fixation member 34, may align with the interior passage 318.

As shown in FIGS. 31B and 33, an upper notch 312 may be defined between the upper tabs 314a and a lower notch 312 may be defined between the lower tabs 314b. Similar to the notches 212, 222 described above, the notches 312 may be configured to receive a portion of the first elongate member 56a and/or the second elongate member 56b during distal advancement of the fixation members 34 within the outer shaft 244.

Namely, similar to that shown in FIG. 29, the distalmost engagement member 78 of the second elongate member 56b may extend distal of the proximal end of the lower tabs 314b and extend into the lower notch 312 between the lower tabs 314b. For example, at least the third portion 78c that extends to the distal end 86 of the engagement member 78 may be positioned within the notch 312 between the lower tabs 314b. The width of the notch 312 may be sized provide clearance between the engagement member 78 and the lower tabs 314b arranged on either side of the engagement member 78. Thus, the engagement member 78 may be deflected into the notch 312 as the fixation member 34 engages and passes by the third portion 78c of the engagement member 78.

Additionally, similar to that shown in FIG. 30, the distalmost engagement member 78 of the first elongate member 56a may extend distal of the proximal end of the upper tabs 314a when actuated to a distal extent of the longitudinal movement of the first elongate member 56a. A portion of the engagement member 78 may extend into the notch 312 between the upper tabs 314a as the engagement member 78 advances distally to deploy the fixation member 34. For example, at least a mid-region of the engagement member 78 may be positioned within the notch 312 between the upper tabs 314a when actuated to a distal extent of the longitudinal movement of the first elongate member 56a. The width of the notch 312 may be sized to provide clearance between the engagement member 78 and the upper tabs 314a arranged on either side of the engagement member 78. Thus, the engagement member 78 may be advanced into the notch 312 as the engagement member 78 advances the fixation member 34 distally of the distal tip 350.

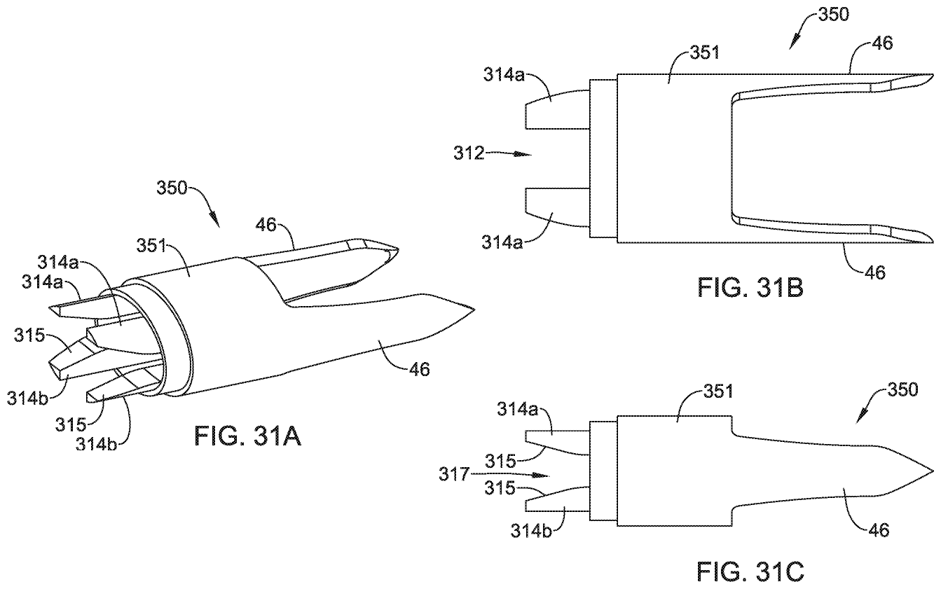

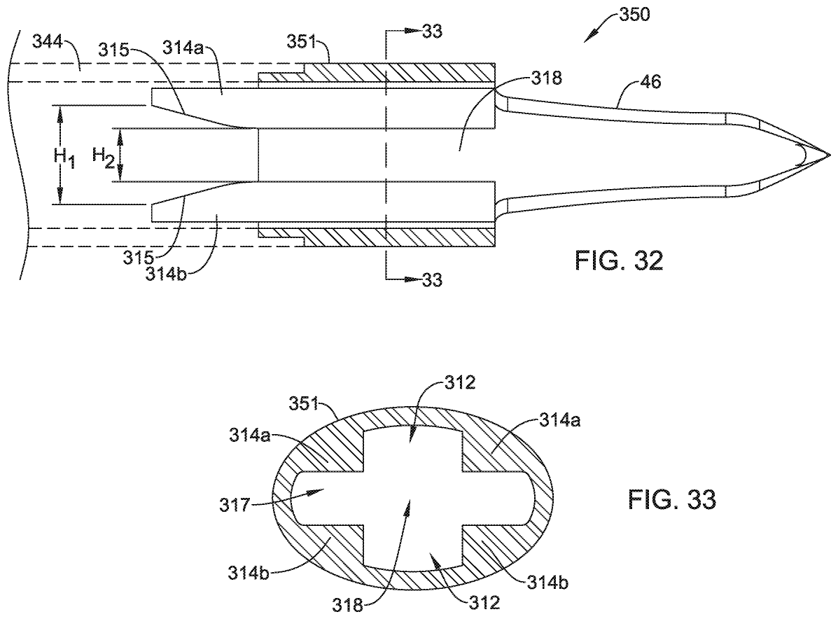

FIG. 34 schematically depicts an alternative shaft assembly 442 for the fixation member delivery system 32, described above. It is noted that the shaft assembly 442 may be attached to the handle 40, and its internal components described above, and extend distally from the handle 40. The shaft assembly 442 may include an outer shaft 444, similar to the outer shaft 44 described above. A pair of tines 46 may be formed at and/or extend from the distal end of the outer shaft 444. As shown in FIG. 34, the pair of tines 46 may be integrally formed at the distal end of the outer shaft 444, however other configurations are contemplated. A plurality of fixation members 34 (not shown) may be loaded within the shaft assembly 442 for sequential deployment therefrom. The shaft assembly 442 may include a first elongate member 56a and a second elongate member 56b (see FIG. 35) extending through the lumen of the outer shaft 444. The first elongate member 56a and the second elongate member 56b may be configured to sequentially advance the fixation members 34 distally within the outer shaft 442 to sequentially deploy a distalmost one of the fixation members 34, as described above. For example, the second elongate member 58b may be held stationary within the lumen of the outer shaft 444 as the first elongate member 58a is reciprocated axially to advance the fixation members 34 distally within the outer shaft 444. Further aspects of the shaft assembly 442 may be similar to the components of the shaft assembly 42 and their interaction with components of the handle 40, described above, and thus will not be repeated.

The outer shaft 444 may include a first, upper crimp 450 and/or a second, lower crimp 452. The upper crimp 450 and the lower crimp 452 may be located at the distal end of the outer shaft 444. The upper crimp 450 and the lower crimp 452 may be portions of the outer shaft 444 in which the annular sidewall of the outer shaft 444 is collapsed inward toward the central longitudinal axis of the outer shaft 444, reducing the distance between opposing sides of the annular sidewall at the crimp(s) 450, 452. As shown in FIG. 35, the distance H between the upper crimp 450 and the lower crimp 452 may be marginally greater than a thickness of the fixation member 34 to permit the fixation member 34 to pass between the upper crimp 450 and the lower crimp 452, while restraining the orientation of the fixation member 34 as the fixation member 34 is advanced distally between the upper crimp 450 and the lower crimp 452.

In some instances, the upper crimp 450 may not be symmetrical to the lower crimp 452. For example, the upper crimp 450 may have a first longitudinal length L1 and the lower crimp 452 may have a second longitudinal length L2, different from the first longitudinal length L1. In some instances, the first longitudinal length L1 of the upper crimp 450 may be greater than the second longitudinal length L2 of the lower crimp 452. However, in other instances, the first longitudinal length L1 of the upper crimp 450 may be less than the second longitudinal length L2 of the lower crimp 452. The different lengths of the upper crimp 450 and the lower crimp 452 may accommodate space within the interior of the outer shaft 444 for deflection of the first elongate member 56a and/or the second elongate member 56b, for example.

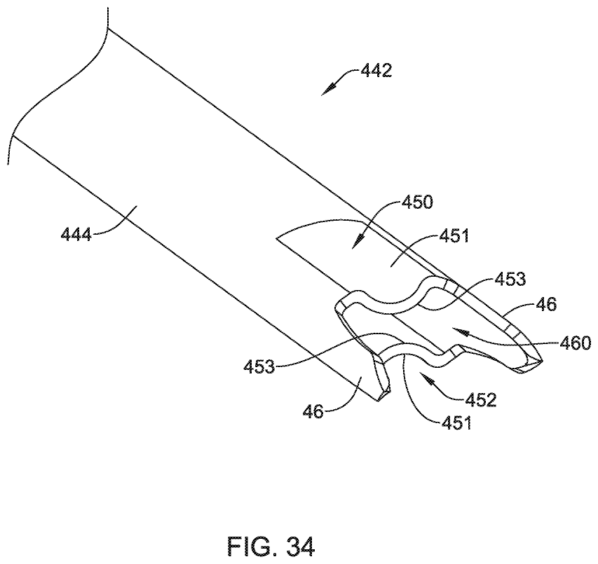

As shown in FIG. 36A, in some embodiments the outer shaft 444 may be a generally oval tubular member, and may have both an upper crimp 450 and a lower crimp 452 extending inward from opposite sides of the outer shaft 444. Each of the upper crimp 450 and the lower crimp 452 may have a concave outer surface 451 on an exterior of the outer shaft 444, and a convex inner surface 453 facing an interior of the outer shaft 444. Thus, the sidewall of the outer shaft 444, defined between the concave outer surface 451 and the convex inner surface 453 may deflect toward the central longitudinal axis of the outer shaft 444 toward the fixation member 34 positioned therebetween.

In an alternative embodiment depicted in FIG. 36B, the outer shaft may be a generally oval tubular member, and may have only one crimp 450 in which the sidewall of the outer shaft 444 is deflected inward toward the central longitudinal axis of the outer shaft 444. Similar to that described above, the crimp 450 may have a concave outer surface 451 on an exterior of the outer shaft 444, and a convex inner surface 453 facing an interior of the outer shaft 444.

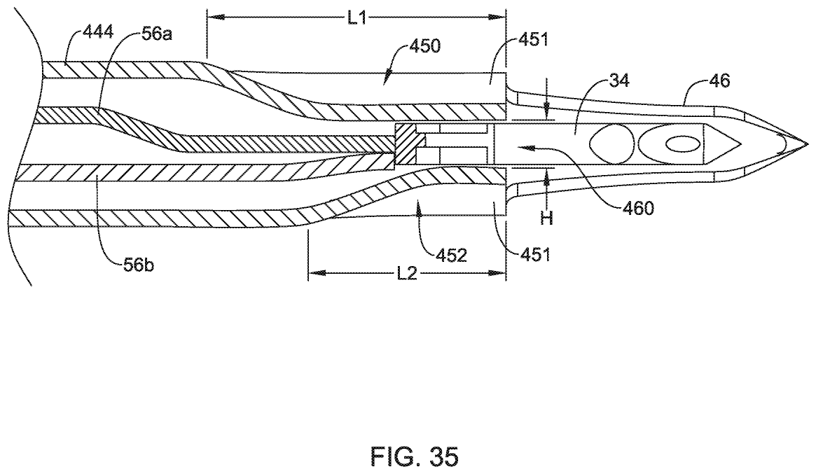

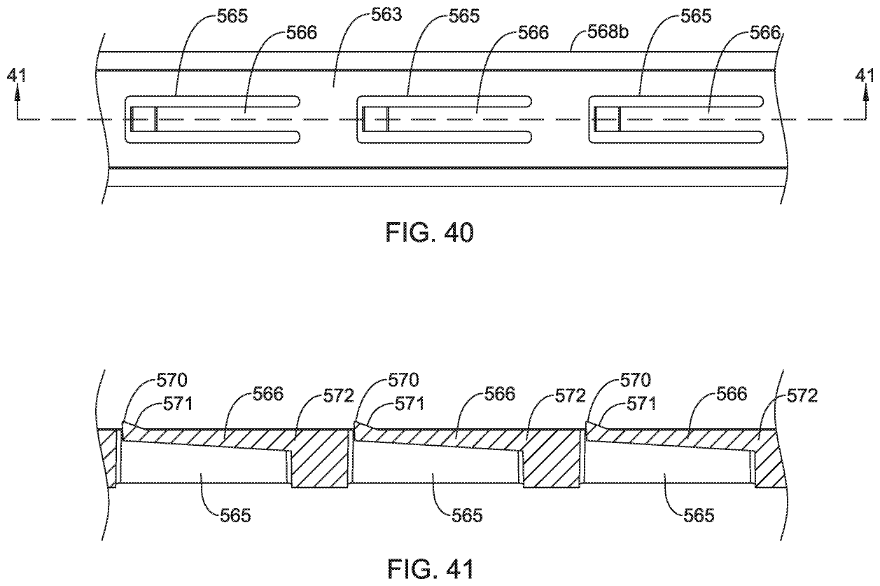

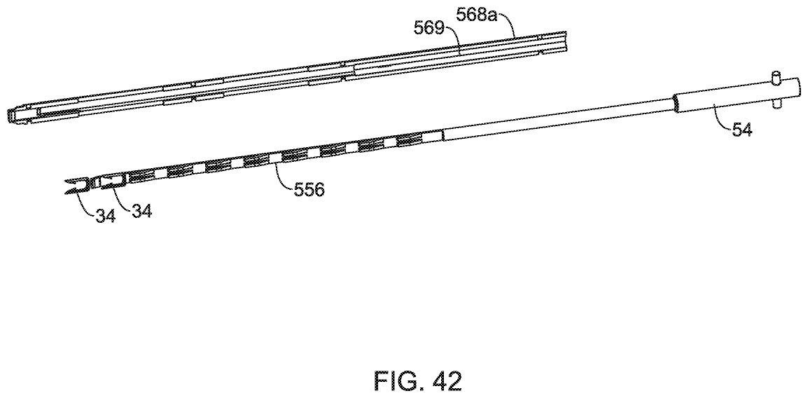

FIG. 37 is an exploded view schematically depicting an alternative shaft assembly 542 for the fixation member delivery system 32, described above. It is noted that the shaft assembly 542 may be attached to the handle 40, and its internal components described above, and extend distally from the handle 40. The shaft assembly 542 may include an outer shaft 544, similar to the outer shaft 44 described above. A pair of tines 46 (provided on a separate distal tip 550) may be formed at and/or extend from the distal end of the outer shaft 544. However, other configurations are contemplated. A plurality of fixation members 34 (see FIGS. 42 and 43) may be loaded within the shaft assembly 542 for sequential deployment therefrom. The shaft assembly 542 may include a first filler component 568a (e.g., a first bumper) and a second filler component 568b (e.g., a second bumper) extending through the lumen of the outer shaft 544. A longitudinally actuatable elongate member 556 (similar to the first elongate member 56a described above) may be positioned between the first filler component 568a and the second filler component 568b. The elongate member 556 may extend proximally of the first filler component 568a and the second filler component 568b and be secured to or otherwise include an elongate shaft 54 couplable to the linkage assembly 52 in the handle 40 for longitudinal actuation by the actuator 50, as described above. As will be further described herein, the shaft assembly 542 may be devoid of a second elongate member 56b, but rather the second filler component 568b may be configured to provide similar functionality as the second elongate member 56b. Further aspects of the shaft assembly 542 may be similar to the components of the shaft assembly 42 and their interaction with components of the handle 40, described above, and thus will not be repeated.

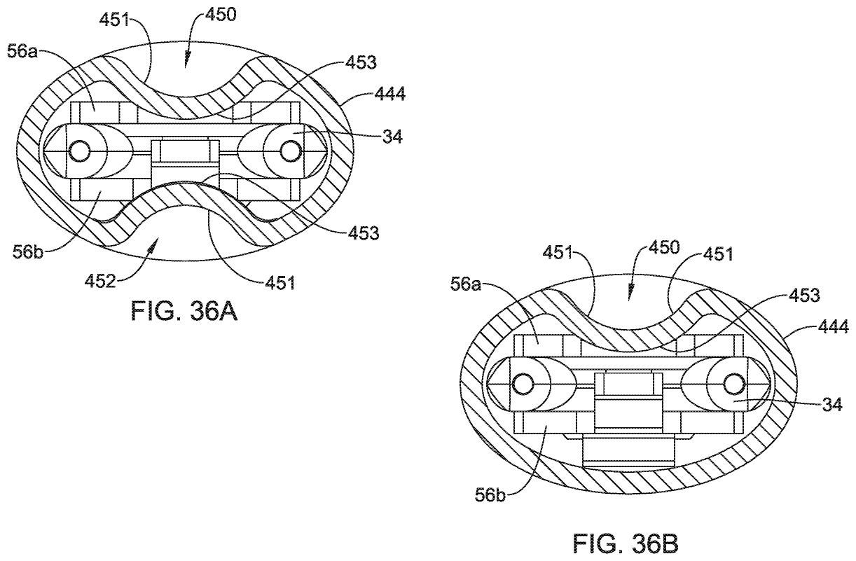

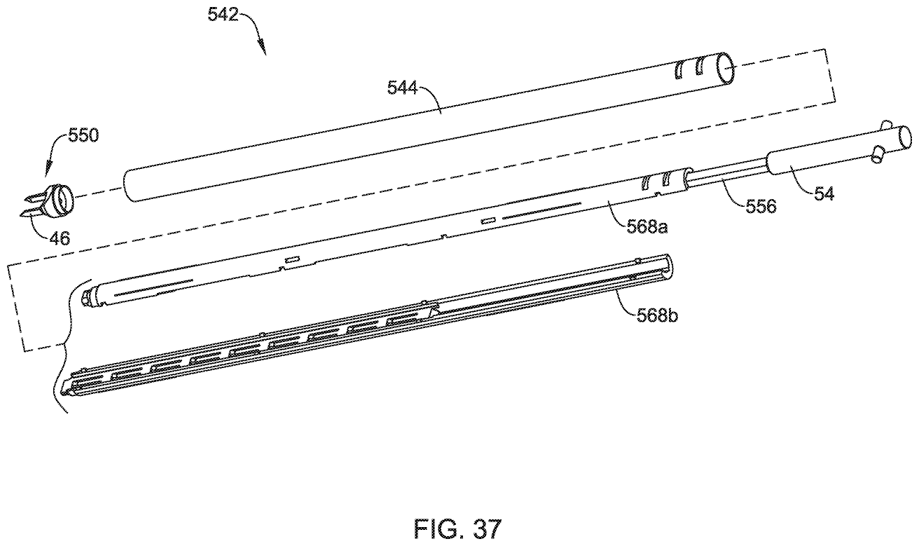

Additional aspects of the second filler component 568b are illustrated in FIGS. 38 and 39. The second filler component 568b has a first, distal end 562 positionable proximate the distal end of the outer shaft 544 and an opposite second, proximal end 564 positionable proximate the proximal end of the outer shaft 544. The second filler component 568b may have a first side, shown in FIG. 38, having a convex surface 567 configured to face an interior surface of the outer shaft 544 when disposed therein. Furthermore, the second filler component 568b may include a plurality of openings 565 uniformly spaced along a length of the second filler component 568b and opening out to the convex surface 567. The plurality of openings 565 may be spaced apart at the same distance that the plurality of fixation members 34 are spaced apart within the shaft assembly 542.

The second filler component 568b may have a second side, shown in FIG. 39, having a surface configured to face the first filler component 568a when disposed within the outer shaft 544. The second filler component 568b may include a cavity 569, such as a concave cavity, configured to slidably receive a portion of the elongate member 556 therein. The second filler component 568b may include a plurality of flexible tabs 566 arranged in the openings 565, thus the flexible tabs 566 may be uniformly spaced along a length of the second filler component 568b and may be spaced apart at the same distance that the plurality of fixation members 34 are spaced apart within the shaft assembly 542.

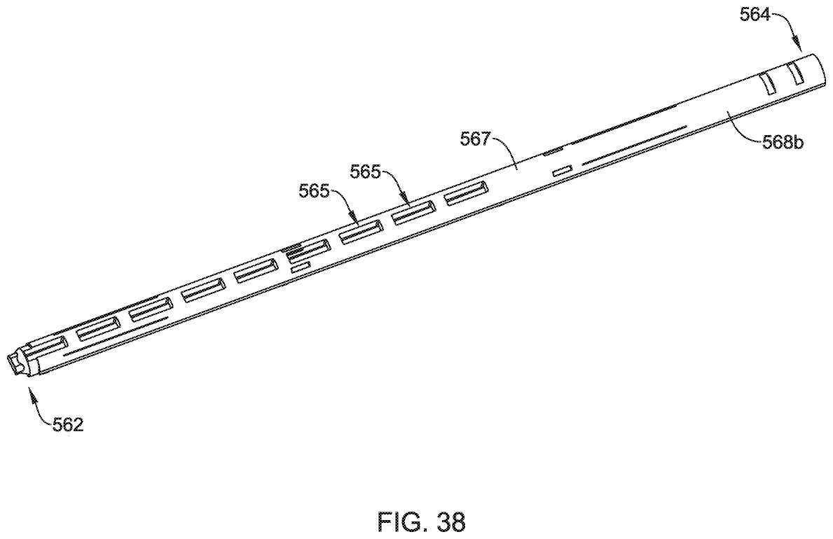

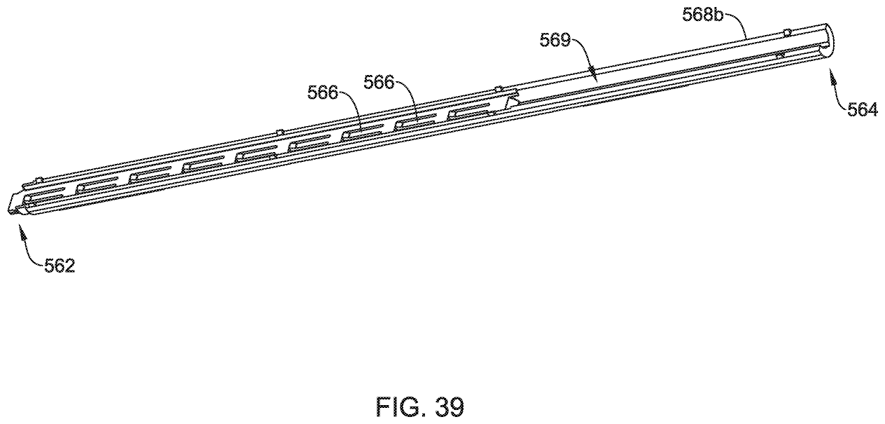

FIG. 40 is an enlarged view of a section of the second filler component 568b and FIG. 41 is a corresponding cross-section view further illustrating the flexible tabs 566 arranged in the openings 565. As shown in FIG. 40, the second side of the second filler component 568b may include a longitudinal track 563 configured to receive the fixation members 34 therealong. The flexible tabs 566 may be centrally arranged along the longitudinal axis of the longitudinal track 563 with the side surfaces of the flexible tabs 566 spaced apart from the sides of the openings 565. A base end 572 of the flexible tabs 566 may be located at the proximal end of the flexible tabs 566, with the flexible tabs 566 extending distally to a distal tip 570. The distal tip 570 may include a ramped surface 571 extending into the longitudinal track 563. As the fixation members 34 are advanced distally, the distal tip 570 of the flexible tabs 566 may be deflected downward into the opening 565 as the fixation members 34 contact or engage the ramped surface 571 and advance distally. The distal tip 570 may provide a lip or abutment surface, preventing a fixation member 34 from retracting back proximally after advancing distally of the distal tip 570.

Additional aspects of the first filler component 568a and the elongate member 556, including the elongate shaft 54 couplable to the linkage assembly 52 in the handle 40 for longitudinal actuation by the actuator 50, are shown in FIG. 42. The first filler component 568a may include a cavity 569, such as a concave cavity, configured to slidably receive a portion of the elongate member 556 therein. A plurality of axially spaced apart fixation members 34 longitudinally advanceable via actuation of the elongate member 556 are also illustrated in FIG. 42.

FIG. 43 is an enlarged view of a distal portion of the elongate member 556 and the fixation members 34 arranged therewith. As shown in FIG. 43, a distal end 86 of the elongate member 556 may be configured to engage a proximal end of the distalmost fixation member 34 to urge the fixation member 34 distally relative to the outer shaft 544 through longitudinal distal movement of the elongate member 556 during deployment of the distalmost fixation member 34. Furthermore, the elongate member 556 may include a plurality of flexible engagement members 578 uniformly arranged along the length of the elongate member 556 which are configured to engage one of the other fixation members 34 (only one of which is shown in FIG. 43) loaded within the shaft assembly 542. Each of the flexible engagement members 578 may have a base end, at a proximal end of the flexible engagement member 578, and an opposite free end extending distally therefrom to a distal end of the flexible engagement member 578. The distal tip of the flexible engagement members 578 may provide an abutment surface contacting and pushing against a fixation member 34 to advance the fixation members 34 distally within the shaft assembly 542. As the elongate member 556 is retracted proximally after advancing the fixation members 34 distally one position, the flexible engagement members 578 may be deflected downward as the flexible engagement members 578 move proximally past the associated fixation member 34.

Both the first filler component 568a and the second filler component 568b may be formed of a polymeric material, such as a thermally non-conductive polymeric material. Furthermore, the elongate member 556, or portions thereof, that directly contact the fixation members 34 may be formed of a polymeric material, such as a thermally non-conductive polymeric material. Forming the first filler component 568a, the second filler component 568b, and/or the elongate member 556 from a thermally non-conductive polymeric material may be advantageous during sterilization of the shaft assembly 542 having a plurality of fixation members 34 loaded therein during the sterilization process. For example, in some instances the fixation members 34 may be formed of a biodegradable polymeric material, such as poly(L, DL-Lactic acid) or PLDL, the material properties and integrity of which can be compromised when exposed to high temperatures. During a sterilization process (e.g., gamma radiation) the absorbed gamma radiation increase the internal energy of the components within the shaft assembly 542. Accordingly, during a sterilization procedure in which the fixation member delivery system 32, or at least the shaft assembly 542 having a plurality of fixation members 34 loaded therein, is subjected to radiation (e.g., gamma radiation), the plurality of fixation members 34 may not be in direct contact with any metallic components, or contact of the plurality of fixation members 34 with metallic components within the shaft assembly 542 may be reduced or minimized, thus reducing the temperature rise of the plurality of fixation members 34.

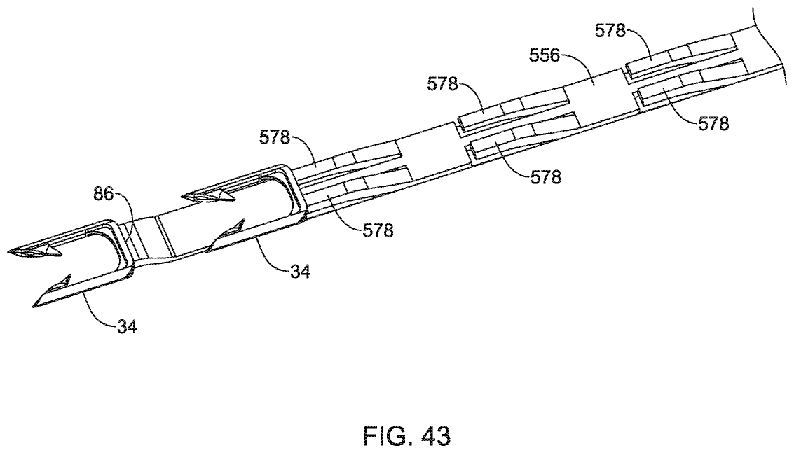

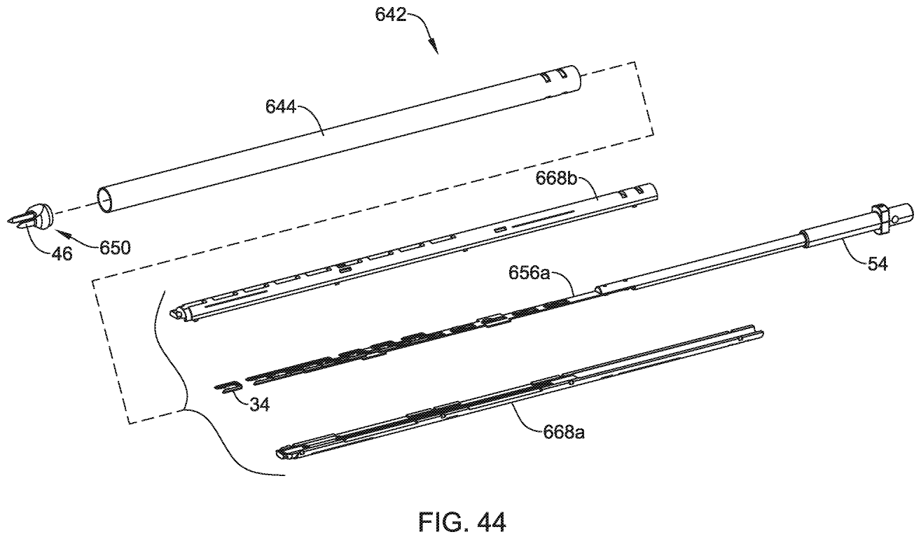

FIG. 44 is an exploded view schematically depicting an alternative shaft assembly 642 for the fixation member delivery system 32, described above. It is noted that the shaft assembly 642 may be attached to the handle 40, and its internal components described above, and extend distally from the handle 40. The shaft assembly 642 may include an outer shaft 644, similar to the outer shaft 44, 544 described above. A pair of tines 46 (provided on a separate distal tip 650) may be formed at and/or extend from the distal end of the outer shaft 644. However, other configurations are contemplated. A plurality of fixation members 34 (see also FIG. 45) may be loaded within the shaft assembly 642 for sequential deployment therefrom. The shaft assembly 642 may include a first filler component 668a (e.g., a first bumper) and a second filler component 668b (e.g., a second bumper) extending through the lumen of the outer shaft 644. A longitudinally actuatable elongate member 656a (similar to the first elongate member 56a described above) may be positioned between the first filler component 668a and the second filler component 668b. The elongate member 656a may extend proximally of the first filler component 668a and the second filler component 668b and be secured to or otherwise include an elongate shaft 54 couplable to the linkage assembly 52 in the handle 40 for longitudinal actuation by the actuator 50, as described above. As will be further described herein, the shaft assembly 642 may be devoid of a second elongate member 56b, but rather the second filler component 668b may be configured similar to the second filler component 568b described above, to provide similar functionality as the second elongate member 56b. Please refer to the description of the second filler component 568b in association with FIGS. 37-41, for details of the second filler component 658b. Further aspects of the shaft assembly 642 may be similar to the components of the shaft assembly 42 and their interaction with components of the handle 40, described above, and thus will not be repeated.

FIG. 45 is an enlarged view of a second of the first filler component 668a and the elongate member 656a, with a plurality of fixation members 34 arranged therealong. The elongate member 656a may be longitudinally slidable relative to the first filler component 668a to sequentially advance the plurality of fixation member 34 through the shaft assembly 642. For example, the elongate member 656a may include one or more, or a plurality of laterally extending slide wings 92 configured to facilitate and/or limit longitudinal adjustment of the first elongate member 656a relative to the first filler component 668a. For example, the first elongate member 656a may include two laterally extending slide wings 92 at one or more (e.g., multiple) axial locations along the first elongate member 656a, but other suitable configurations are contemplated. In some examples, the slide wings 92 may be aligned with and/or received in longitudinally extending channels or slots 94 of the first filler component 668a to facilitate and limit proximal and distal movement of the first elongate member 656a. When the second filler component 668b is positioned along the first filler component 668a, the second filler component 668b may cover or otherwise act as a cover for the slots 94 to secure the slide wings 92 within the slots 94. The first elongate member 656a may include one or more additional or alternative longitudinal adjustment facilitating/limiting components, as desired.

The first elongate member 656a may include may include a plurality of flexible engagement members 78 uniformly arranged along the length of the elongate member 656a which are configured to engage one of the other fixation members 34 loaded within the shaft assembly 642. Each of the flexible engagement members 78 may have a base end, at a proximal end of the flexible engagement member 78, and an opposite free end extending distally therefrom to a distal end of the flexible engagement member 78. The distal tip of the flexible engagement members 78 may provide an abutment surface contacting and pushing against a fixation member 34 to advance the fixation members 34 distally within the shaft assembly 642. As the elongate member 656a is retracted proximally after advancing the fixation members 34 distally one position, the flexible engagement members 78 may be deflected downward as the flexible engagement members 78 move proximally past the associated fixation member 34.

Both the first filler component 668a and the second filler component 668b may be formed of a polymeric material, such as a thermally non-conductive polymeric material. Furthermore, the elongate member 656a, or portions thereof, that directly contact the fixation members 34 may be formed of a polymeric material, such as a thermally non-conductive polymeric material, or the elongate member 656a may be formed of a metallic material, if desired. Forming the first filler component 568a and the second filler component 568b (and in some cases the elongate member 656a) from a thermally non-conductive polymeric material may be advantageous during sterilization of the shaft assembly 642 having a plurality of fixation members 34 loaded therein during the sterilization process. For example, in some instances the fixation members 34 may be formed of a biodegradable polymeric material, such as poly(L, DL-Lactic acid) or PLDL, the material properties and integrity of which can be compromised when exposed to high temperatures. During a sterilization process (e.g., gamma radiation) the absorbed gamma radiation increase the internal energy of the components within the shaft assembly 642. Accordingly, during a sterilization procedure in which the fixation member delivery system 32, or at least the shaft assembly 642 having a plurality of fixation members 34 loaded therein, is subjected to radiation (e.g., gamma radiation), the plurality of fixation members 34 may not be in direct contact with any metallic components, or contact of the plurality of fixation members 34 with metallic components within the shaft assembly 542 may be reduced or minimized to only the contact with the distal tips of the flexible engagement members 78, thus reducing the temperature rise of the plurality of fixation members 34.

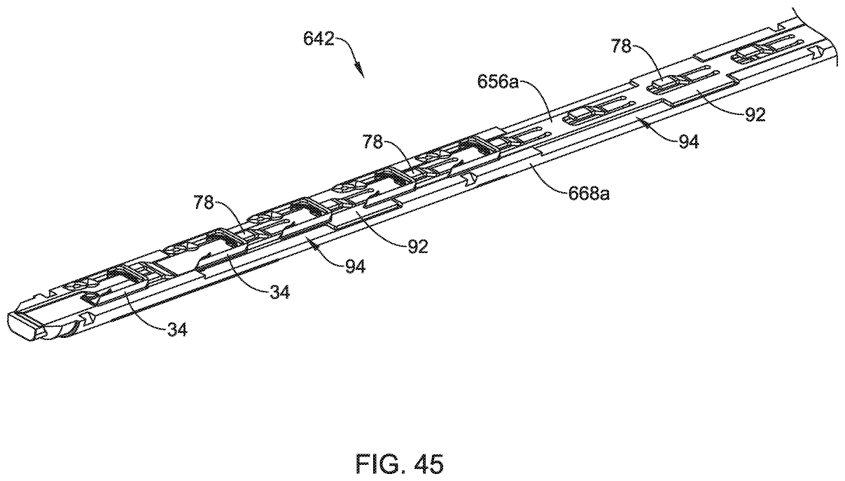

FIG. 46 depicts an alternative fixation member delivery system 732 including an attachable/detachable shaft assembly 742 which is attachable to and/or detachable from a handle 748. The shaft assembly 742 may be similar to any of the shaft assemblies described above, including internal components for sequentially advancing a plurality of fixation members 34 within the shaft assembly 742 for deployment therefrom. Accordingly, further aspects of the shaft assembly 742 may be combined with any of the shaft assemblies described above, if desired.

Furthermore, the handle 748 may be similar to any of the handles described above, including internal components for actuating the internal components of the shaft assembly 742 to advance fixation members 34 loaded therein. For example, the handle 748 may include a first housing member 748a and a second housing member (not shown) housing a linkage assembly therein. The linkage assembly may be actuatable with an actuator 50 for actuating the internal components of the shaft assembly 742 to advance fixation members 34 loaded therein.

The attachable/detachable shaft assembly 742 may be configured to be coupled to and/or uncoupled from the handle 748. For example, the attachable/detachable shaft assembly 742 may be disposable, whereas the handle 748 may be reusable. In some instances, the shaft assembly 742 may be pre-loaded with a plurality of fixation members 34 (such as pre-loaded with 6-8 fixation members 34), and subsequently coupled to the handle 748. Upon deploying the last of the fixation members 34 from the shaft assembly 742, the shaft assembly 742 may be decoupled from the handle 748 and another shaft assembly 742 (having another plurality of pre-loaded fixation members 34 loaded therein) may be coupled to the handle 748 for subsequent use during a medical procedure. In other instances, the shaft assembly 742, having a plurality of pre-loaded fixation members 34 loaded therein) may be subjected to a sterilization process prior to being coupled to the handle 748, and thereafter coupled to the handle 748 for use in a medical procedure. Other advantages of providing an attachable/detachable shaft assembly 742 that is attachable to and/or detachable from the handle 748 are also contemplated.

The shaft assembly 742 includes an outer shaft 744 and an elongate shaft 754 longitudinally slidable within the outer shaft 744 through actuation of the actuator 50 to deploy fixation members therefrom. The elongate shaft 754 may be similar to the elongate shaft 54 described with the various embodiments above, connected to an elongate member configured to distally advance the plurality of fixation members with the shaft assembly 742.

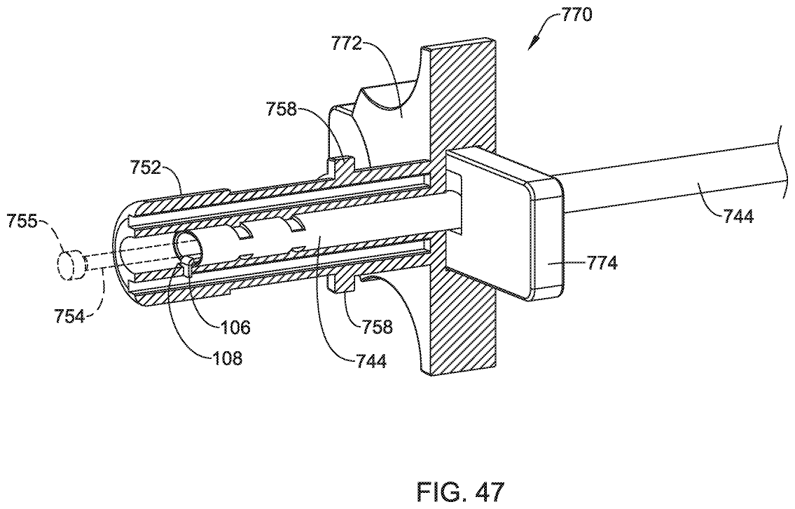

The shaft assembly 742 may also include a connection interface configured to be coupled to a mating connection interface of the handle 748. For example, the shaft assembly 742 may include an elongate post 752 configured to extend into a distal opening 780 in the handle 748. One or more, or a plurality of tabs 758 may extend radially outward from the elongate post 752 for insertion into a corresponding channel 782 formed in the distal opening 780. It is noted that only one channel 782 is shown in FIG. 46, but it is contemplated that the second housing member (not shown) could have a corresponding second channel 782. The tabs 758 may be advanced proximally along the corresponding channels 782 as the elongate post 752 is advanced proximally into the distal opening 780, and thereafter the elongate post 752, and the remainder of the shaft assembly 742, may be rotated (e.g., rotated 90 degrees, rotated 180 degrees, etc.) to lock the shaft assembly 742 to the handle 748 as the tabs 758 are rotationally moved into transverse portions of the channels 782. Furthermore, as the elongate post 752 is advanced into the distal opening 780, the proximal end of the elongate shaft 754 may be advanced into the interior of the spring 60 to engage a push rod 750 (see FIG. 48) of the linkage within the handle 748.

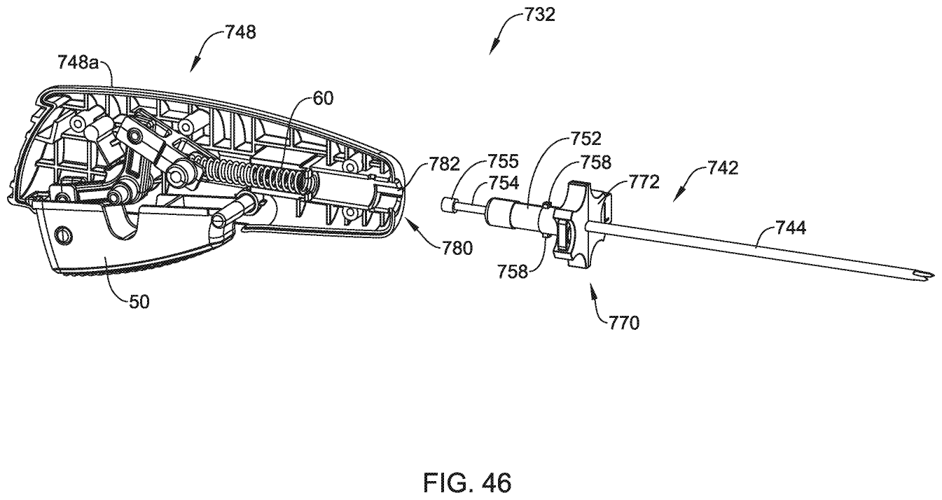

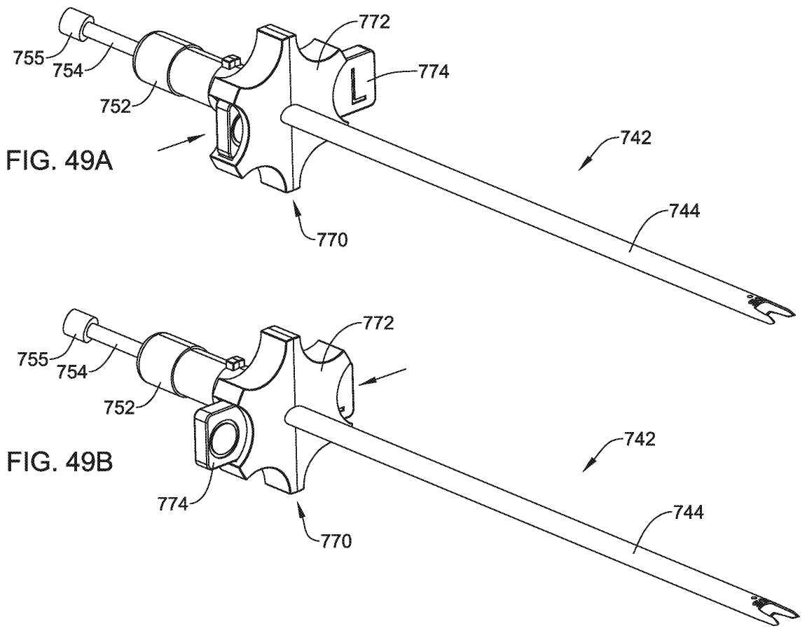

Further aspects of the shaft assembly 742 are shown in FIGS. 47 and 50. For example, the proximal end of the outer shaft 744 may extend into the interior of the elongate post 752 and be secured thereto. Furthermore, the tab 106 formed at the proximal end of the second elongate member 56b (see discussion above) may be inserted into a slot 108 formed in the wall of the elongate post 752 to hold the second elongate member 56b stationary relative to the outer shaft 744 and the elongate post 752.

The elongate shaft 754 may extend proximal of the outer shaft 744 and include a coupling for connecting to the push rod 750 in the handle 748. For example, in the embodiment of FIGS. 46-48, the proximal end of the elongate shaft 754 may include a magnet 755 (or magnetic material) configured to magnetically couple to a magnet 757 (or magnetic material) provided at the distal end of the push rod 750 to couple the elongate shaft 754 to the push rod 750. Some alternative coupling mechanisms are described below. The magnetic connection between the magnet 755 and the magnet 757 may be great enough to proximally retract the elongate shaft 754 proximally when the actuator 50 is released without breaking the magnetic connection therebetween.

The shaft assembly 742 may also include a lock mechanism 770 configured to selectively lock axial movement of the elongate shaft 754 (and thus the movable elongate member within the outer shaft 744) relative to the outer shaft 744. Such a lock mechanism 770 may be used to prevent inadvertent deployment of a fixation member 34 from the shaft assembly 742 during coupling and/or decoupling the shaft assembly 742 to the handle 748, and/or during use of the fixation member delivery system 732.

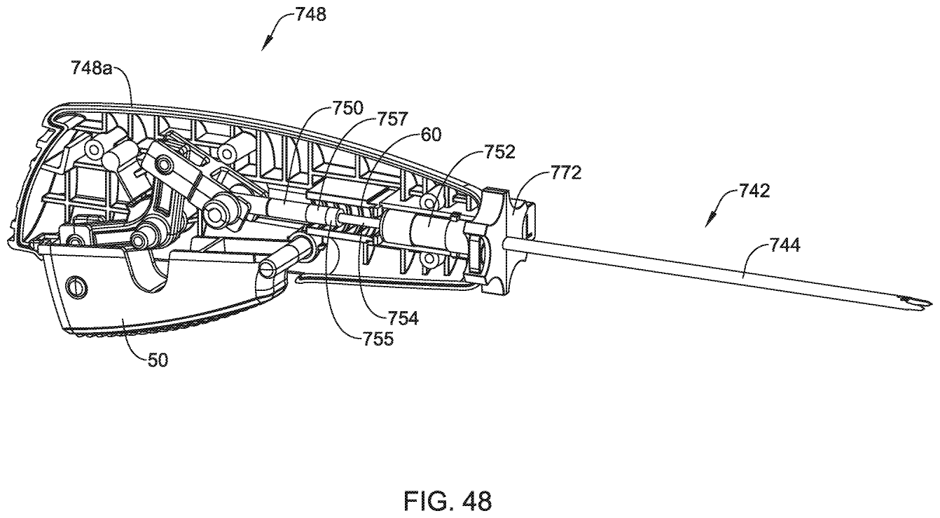

Turning to FIG. 47, the lock mechanism 770 may include a housing 772 and an actuator 774 (e.g., slider, button, latch, etc.) movable relative to the housing 772 to selectively lock movement of the elongate shaft 754. For example, as shown in FIGS. 49A and 49B, actuator 774 may be movable between a locked position (FIG. 49A) and an unlocked position (FIG. 49B). As shown by the arrows in FIGS. 49A and 49B, the actuator 774 may be moveable in a direction perpendicular to the longitudinal axis of the outer shaft 744 between the locked and unlocked positions, for example. The actuator 774 may include visible indicia (coloration, symbols, letters, etc.) that may be visible to the operator to discern whether the lock mechanism 770 is in the locked position or the unlocked position.

In one configuration, as shown in FIG. 50, the actuator 774 may move into or engaged with a slot 775 (or other structure) of the elongate shaft 754 when in the locked position, thus not permitting axial movement of the elongate shaft 754 relative to the outer shaft 744. The actuator 774 may be moved out of tor disengaged from the slot 775 (or other structure) of the elongate shaft 754 when in the unlocked position, thus permitting axial movement of the elongate shaft 754 relative to the outer shaft 744.

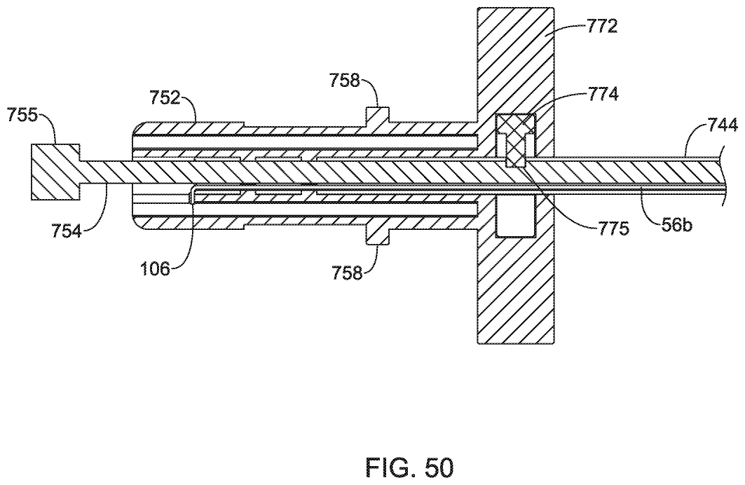

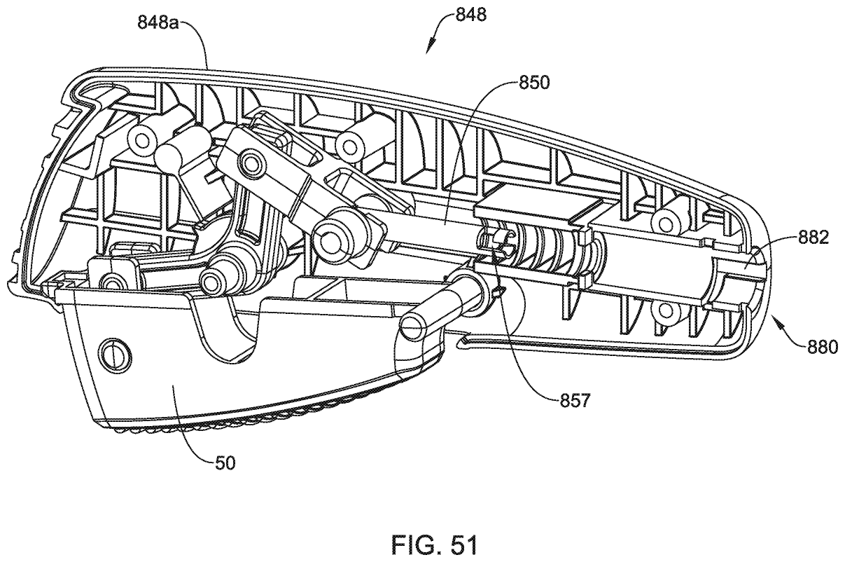

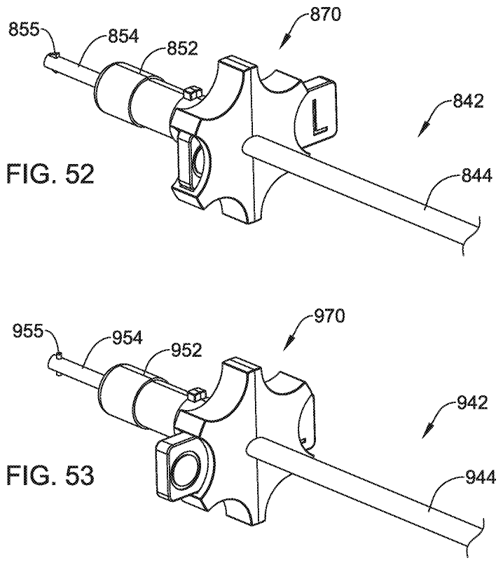

An alternative handle 848 including an alternative coupling interface for coupling to/decoupling from an attachable/detachable shaft assembly 842, 942 (shown in FIGS. 52 and 53) is shown in FIG. 51. The handle 848 may be similar to any of the handles described above, including internal components for actuating the internal components of the shaft assembly 842, 942 to advance fixation members 34 loaded therein. For example, the handle 848 may include a first housing member 848a and a second housing member (not shown) housing a linkage assembly therein. The linkage assembly may be actuatable with an actuator 50 for actuating the internal components of the shaft assembly 842, 942 to advance fixation members 34 loaded therein.

The shaft assembly 842, 942 includes an outer shaft 844, 944 and an elongate shaft 854, 954 longitudinally slidable within the outer shaft 844, 944 through actuation of the actuator 50 to deploy fixation members therefrom. The elongate shaft 854, 954 may be similar to the elongate shaft 54 described with the various embodiments above, connected to an elongate member configured to distally advance the plurality of fixation members with the shaft assembly 842, 942.

The shaft assembly 842, shown in FIG. 52, and the shaft assembly 942, shown in FIG. 53, may include a lock mechanism 870, 970, respectively, similar to the lock mechanism 770 described above. Accordingly, details of the lock mechanism 870, 970 and its implementation will not be repeated, but are equally applicable to the shaft assembly 842 and the shaft assembly 942.

The shaft assembly 842, shown in FIG. 52, and the shaft assembly 942, shown in FIG. 53, may also include a connection interface configured to be coupled to a mating connection interface of the handle 848. For example, the shaft assembly 842, 942 may include an elongate post 852, 952 configured to extend into a distal opening 880 in the handle 848. One or more, or a plurality of tabs may extend radially outward from the elongate post 852, 952 for insertion into a corresponding channel 882 formed in the distal opening 880. It is noted that only one channel 882 is shown in FIG. 51, but it is contemplated that the second housing member (not shown) could have a corresponding second channel 882. The tabs may be advanced proximally along the corresponding channels 882 as the elongate post 852, 952 is advanced proximally into the distal opening 880, and thereafter the elongate post 852, 952, and the remainder of the shaft assembly 842, 942, may be rotated (e.g., rotated 90 degrees, rotated 180 degrees, etc.) to lock the shaft assembly 842, 942 to the handle 848 as the tabs are rotationally moved into transverse portions of the channels 882. Furthermore, as the elongate post 852, 952 is advanced into the distal opening 880, the proximal end of the elongate shaft 854, 954 may be advanced into the interior of the spring (not shown for clarity, but see spring 60 described above) to engage a push rod 850 (see FIG. 51) of the linkage within the handle 848.

The elongate shaft 854, 954 may extend proximal of the outer shaft 844, 944 and the elongate post 852, 952, and include a coupling for connecting to the push rod 850 in the handle 848. For example, in the embodiment of FIG. 52, the proximal end of the elongate shaft 854 may include one or more, or a plurality of tabs 855 extending radially outward from the elongate shaft 854. The tab(s) 855 may be configured to extend into and/or otherwise engage a slot 857 (see FIG. 51) or other structure formed at the distal end of the push rod 850. In some instances, the tab(s) 855 may have a tapered leading edge to facilitate guiding the tab(s) 855 into the slot 857 of the push rod 850.

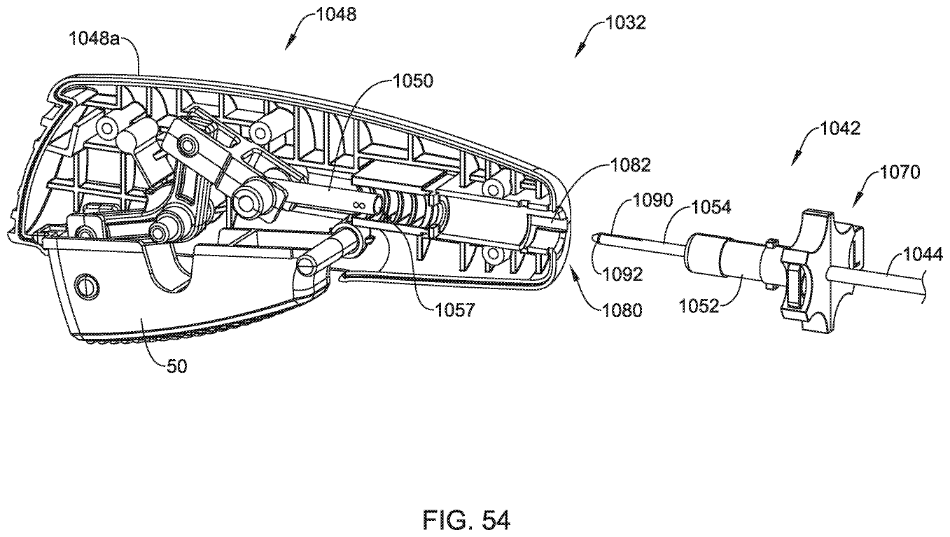

In the embodiment of FIG. 53, the proximal end of the elongate shaft 854 may include a pin 955 extending radially outward from the elongate shaft 954. The pin 955 may extend outward in one direction on one side of the elongate shaft 954, or the pin 955 may extend outward in opposite directions from opposite sides of the elongate shaft 954, for example. The pin 955 may be configured to extend into and/or otherwise engage the slot 857 (see FIG. 51) or other structure formed at the distal end of the push rod 850. FIG. 54 depicts an alternative fixation member delivery system 1032, including an alternative handle 1048 with an alternative coupling interface for coupling to an attachable shaft assembly 1042. The handle 1048 may be similar to any of the handles described above, including internal components for actuating the internal components of the shaft assembly 1042 to advance fixation members 34 loaded therein. For example, the handle 1048 may include a first housing member 1048a and a second housing member (not shown) housing a linkage assembly therein. The linkage assembly may be actuatable with an actuator 50 for actuating the internal components of the shaft assembly 1042 to advance fixation members 34 loaded therein.

The shaft assembly 1042 includes an outer shaft 1044 and an elongate shaft 1054 longitudinally slidable within the outer shaft 1044 through actuation of the actuator 50 to deploy fixation members therefrom. The elongate shaft 1054 may be similar to the elongate shaft 54 described with the various embodiments above, connected to an elongate member configured to distally advance the plurality of fixation members with the shaft assembly 1042. The shaft assembly 1042 may include a lock mechanism 1070 similar to the lock mechanism 770 described above. Accordingly, details of the lock mechanism 1070 and its implementation will not be repeated, but are equally applicable to the shaft assembly 1042.

The shaft assembly 1042 may also include a connection interface configured to be coupled to a mating connection interface of the handle 1048. For example, the shaft assembly 1042 may include an elongate post 1052 configured to extend into a distal opening 1080 in the handle 1048. One or more, or a plurality of tabs may extend radially outward from the elongate post 1052 for insertion into a corresponding channel 1082 formed in the distal opening 1080. It is noted that only one channel 1082 is shown in FIG. 54, but it is contemplated that the second housing member (not shown) could have a corresponding second channel 1082. The tabs may be advanced proximally along the corresponding channels 1082 as the elongate post 1052 is advanced proximally into the distal opening 1080, and thereafter the elongate post 1052 and the remainder of the shaft assembly 1042 may be rotated (e.g., rotated 90 degrees, rotated 180 degrees, etc.) to lock the shaft assembly 1042 to the handle 1048 as the tabs are rotationally moved into transverse portions of the channels 1082. Furthermore, as the elongate post 1052 is advanced into the distal opening 1080, the proximal end of the elongate shaft 1054 may be advanced into the interior of the spring (not shown for clarity, but see spring 60 described above) to engage a push rod 1050 of the linkage within the handle 1048.

The elongate shaft 1054 may extend proximal of the outer shaft 1044 and the elongate post 1052, and include a coupling for connecting to the push rod 1050 in the handle 1048. For example, the proximal end region of the elongate shaft 1054 may include at least one slot 1090 extending distally from a proximal end of the elongate shaft 1054, dividing the proximal end region of the elongate shaft 1054 into a plurality of flexible legs. The elongate shaft 1054 may also include a protrusion 1092 extending radially outward therefrom. The protrusion 1092 may be a knob, bump, tooth, annular rib, or other structure deviating outward from the outer surface of the elongate shaft 1054. The proximal end region of the elongate shaft, including the protrusion 1092 may be inserted into the lumen 1057 of the push rod 1050 as the shaft assembly 1042 is being coupled to the handle 1048. The protrusion 1092 may engage the distal rim of the push rod 1050 as the proximal end region of the elongate shaft 1054 is advanced into the lumen 1057, causing the flexible legs to deflect radially inward toward one another to allow clearance of the protrusion 1092 into the lumen 1057. The elongate shaft 1054 may include a notch, interior recess, internal lip, etc. configured to receive the protrusion 1092 to thereby lock the elongate shaft 1054 into engagement with the push rod 1050. The connection between the protrusion 1092 and the mating structure of the push rod 1050 may be great enough to distally actuate the elongate shaft 1054 when the actuator 50 is engaged (e.g., squeezed) and proximally retract the elongate shaft 1054 proximally when the actuator 50 is released without breaking the connection therebetween.

In some instances, the connection between the elongate shaft 1054 and the push rod 1050 may be non-reversable such that the elongate shaft 1054 cannot be thereafter decoupled from the push rod 1050 without damaging one or more of the elongate shaft 1054 and the push rod 1050. In other instances, the connection between the elongate shaft 1054 and the push rod 1050 may be reversable such that the elongate shaft 1054 can later be decoupled form the push rod 1050, if desired.

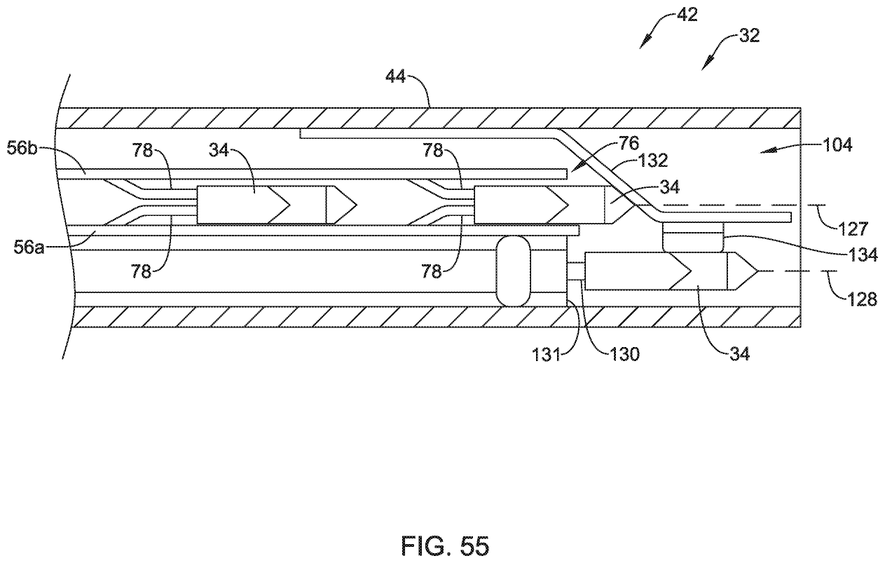

FIG. 55 schematically depicts components of a distal end of an illustrative configuration of the delivery system 32, where the outer shaft 44 is sectioned and the contents of the outer shaft are shown in side view. As depicted in FIG. 55, the depicted delivery system 32 may include a dual axis or plane system having a storage axis for advancing the fixation members 34 in the distal direction and a deployment axis.

A handle of the illustrative configuration of the delivery system 32 depicted in FIG. 55 may include a handle configured similar to the handle 40 discussed herein, where the actuator 50 (e.g., a first actuator 50a) is configured to be actuated to advance the fixation members 34 in the distal direction and from a storage plane or axis 127 to a deployment plane or axis 128. A further actuator or lever (e.g., a second actuator 50b) may be engaged to deploy the distal-most fixation member 34 out of the distal end of the delivery system 32. In some configurations, however, a single actuator may be utilized to advance the fixation members 34 and deploy the fixation members 34. Other suitable configurations of the handle 40 are contemplated.

As depicted in FIG. 55, the delivery system 32 may include the fixation member actuation assembly 76 along the fixation member storage plane or axis 127, with the first elongate member 56a having engagement members 78 and the second elongate member 56b having engagement members 78. The fixation member actuation assembly 76 may operate as discussed herein with respect to the illustrative delivery system 32 depicted in FIG. 4, as discussed herein with respect to the illustrative delivery system 32 depicted in FIG. 17, and/or in accordance with the operation of one or more other suitable delivery systems configured to advance one or more fixation members 34 in the distal direction. For example, the first elongate member 56a may be advanced along the storage axis 127 to advance a plurality of fixation members 34 in the distal direction along the storage axis 127, while the second elongate member 56b remains stationary, as discussed herein. Alternatively or additionally, the fixation member actuation assembly 76 including the first elongate member 56a and the second elongate member 56b may operate similar to the components configured to advance and/or deploy anchors or staples in U.S. Patent Application Publication No. 2023/0097234A 1 , titled MEDICAL IMPLANT DELIVERY SYSTEM AND RELATED METHODS, filed Nov. 14, 2022, which is hereby incorporated by reference in its entirety for any and all purposes.

The configuration of the delivery system 32 depicted in FIG. 55 may include a deployment mechanism 130 along a deployment axis 128. In response to being actuated (e.g., via the second actuator 50b, such as a lever, actuator, push member, etc. at or proximate the handle 40), the deployment mechanism 130 may be configured to deploy fixation members 34 that have been moved from the storage axis 127 to the deployment axis 128. In some examples, the deployment mechanism 130 may engage the proximal end 88 and/or other portions of the fixation member 34 and push the fixation member 34 out of the outer shaft 44 and into the implant 12 and tissue of the patient. In one example, the deployment mechanism 130 may be or may include a push rod or shaft extending distally from the handle 40 and configured to engage and deploy the fixation member(s) 34 along the deployment axis 128 in response to actuation of the push rod or shaft. The deployment mechanism 130 may extend from the handle 40 to the distal end of the shaft assembly 42, such that the deployment mechanism 130 may be actuated by a clinician to deploy the distal-most fixation member 34. Other suitable configurations of the deployment mechanism 130.

The deployment mechanism 130 may be positioned entirely or at least partially within an elongate housing 131, such that the deployment mechanism 130 may adjust longitudinally relative to the housing 131. In some examples, the housing 131 may be an elongate tube, an elongate shaft, a portion of the outer shaft 44, and/or other suitable component. Alternatively or additionally, the housing 131 may be at least partially defined by the outer shaft 44.

The configuration of the delivery system 32 depicted in FIG. 55 may include a transfer component configured to facilitate the fixation members 34 moving from a position along the storage axis 127 to a position along the deployment axis 128. The transfer component may be any suitable component configured to facilitate moving the fixation members 34 from the storage axis 127 to the deployment axis 128 including, but not limited to, a ramped surface, an actuatable push member, a spring, flat spring, a torsion spring, a leaf spring, a coiled spring, a V-shaped flat spring, a cam spring, and/or other suitable components configured to move the fixation members 34 laterally from the storage axis 127 to the deployment axis 128. In some examples, the transfer component may be a transfer spring 132. In one example, the transfer spring 132 may be configure to make contact with a portion of the fixation member 34 proximal to a distal tip of the fixation member 34 such that the tip of the fixation member 34 does not touch the transfer spring 132 as the tissue anchor is moved in the distal direction, which may prevent dulling of the distal tip of the fixation member 34.

When the transfer component may be or may include the transfer spring 132, the transfer spring 132 may be adjusted between a resting position at which fixation members 34 are blocked from moving to the deployment axis 128 and a stressed position. The transfer spring 132 may be biased to the resting position, as depicted in FIG. 55.

In operation, as a distal-most fixation member 34 is advanced distally along the storage axis 127, the distal-most fixation member 34 may engage the transfer spring 132 and the force used to advance the fixation members 34 in the distal direction may overcome the bias of the transfer spring 132 while the fixation member 34 remains within the fixation member actuation assembly 76 and move the transfer spring 132 outward to a stressed position. As more of the distal-most fixation member 34 exits the fixation member actuation assembly 76, the bias of the transfer spring 132 may act on the distal-most fixation member 34 to move the fixation member 34 from the storage axis 127 to the deployment axis 128 and engagement with the deployment mechanism 130. The deployment mechanism 130 may be actuated and advanced in the distal direction to deploy the fixation member 34 positioned along the deployment axis 128. After deploying the fixation member 34, the deployment mechanism 130 may be cycled in the proximal direction and then the distal direction to reload a fixation member 34 along the deployment axis 128 for deployment.

The transfer component, such as the transfer spring 132, may include a stop component 134. The stop component 134 may have any suitable shape and/or configuration designed to maintain the fixation member 34 in the shaft assembly 42 (e.g., maintain the fixation member 34 in the shaft assembly 42 as the deployment mechanism 130 advances in the distal direction and engages the fixation member 34). In some examples, the stop component 134 may be a protuberance that engages a distal side of the bridge between the arms of the fixation member 34. In some examples, the stop component 134 may have a convex shape with rounded surfaces, such that the stop component 134 may maintain the fixation member 34 within the shaft assembly 42 while also facilitating advancement of the fixation member 34 for deployment when a force in the distal direction is applied to the fixation member 34 by the deployment mechanism 130 sufficient to overcome a bias of the transfer spring 132 or other suitable transfer component and causing the transfer spring 132 or other suitable transfer component to adjust or deflect laterally. Other suitable configurations of the stop component 134 are contemplated.

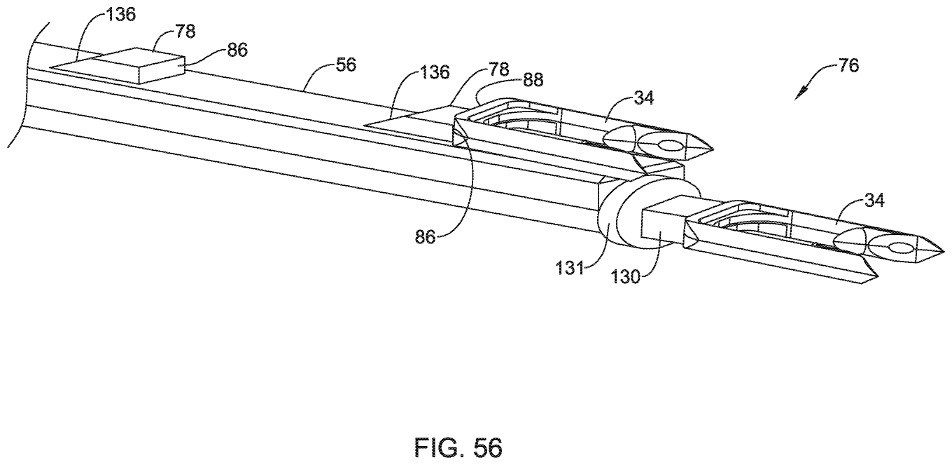

FIG. 56 schematically depicts a perspective view of a distal end of an illustrative configuration of the delivery system 32, where the outer shaft 44 has been removed for clarity. The delivery system 32 may have a similar configuration to the configuration of the delivery system 32 depicted in FIG. 55, but with a single elongate member 56 with engagement members 78 extending therefrom that is configured to advance in the distal direction and the proximal direction relative to the housing 131, the outer shaft 44, and/or other components of the shaft assembly 42.

The engagement members 78 depicted in FIG. 56 may have a fixed configuration and may not be designed to flex in response to engagement with the fixation members 34. For example, the engagement members 78 of the elongate member 56 may have the distal end 86 configured to engage a proximal end 88 of the fixation member 34 and a ramped surface 136 extending from a base of the elongate member 56. In some examples, there may be sufficient space between the elongate member 56 and the outer shaft 44 such that when the ramped surface engages the fixation member 34, the fixation member 34 may adjust laterally outward within the outer shaft 44 to allow the engagement member 78 to pass the fixation member 34 during the actuation cycle.

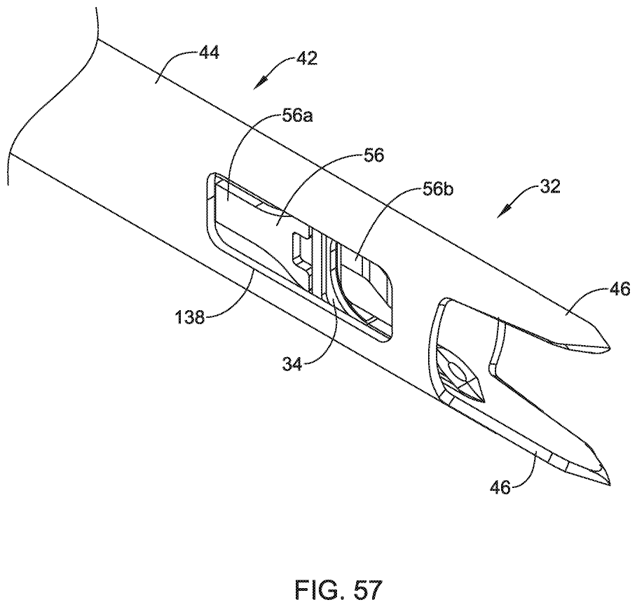

FIG. 57 schematically illustrates a distal end of a shaft assembly 42 of an illustrative fixation member delivery system 32 configured to insert fixation members 34 through an implant into tendon tissue and/or bone of a patient. In some examples, the delivery system 32 may function similar to the delivery systems 32 discussed herein and/or similar to other suitable fixation member delivery systems.

As depicted in FIG. 57, the outer shaft 44 may include an opening or window 138 proximate a distal end of the outer shaft 44 and proximal of the tines 46. The window 138 may allow a physician to confirm the distal-most fixation member 34 is in a position to be deployed prior to, during, and/or after deployment of the fixation member 34. Additionally or alternatively, the window 138 may allow a physician to confirm a movable elongate member 56 (e.g., the first elongate member 56a) is engaging a proximal end of the fixation member 34 and/or is otherwise in position to deploy the distal-most fixation member 34 when actuated.

The shaft assembly 42 may include any suitable number of windows 138. For example, the outer shaft 44 may include one or windows 138 spaced along the length of the outer shaft 44 and/or one or more windows 138 circumferentially spaced around the outer shaft 44. In one example, a single window 138 may be positioned proximate the distal end of the outer shaft 44 and adjacent the movable elongate member 56, such that at least the distal most fixation member 34 and the movable elongate member 56 may be viewed through the window 138, as depicted in FIG. 57. In another example, a single window 138 may be positioned proximate the distal end of the outer shaft 44 at a circumferential location between and/or spanning a space between the first elongate member 56a and the second elongate member 56b, such that at least the distal-most fixation member 34, the first elongate member 56a, and/or the second elongate member 56b may be viewed through the window 138. In another example, a plurality of windows 138 may be spaced along the length of the outer shaft 44, which may facilitate determining a total number of fixation members 34 loaded into the shaft assembly 42 and/or that the loaded fixation members 34 are in position for advancement relative to the elongate member(s) 56. In another example, a plurality of windows 138 may be spaced circumferentially around the outer shaft 44, which may facilitate confirming the fixation member(s) 34 are positioned in a desired manner relative to the elongate member(s) 56.

The windows 138 may have any suitable shape and/or size. For example, the windows 138 may have a size and/or shape including, but not limited to, an oval shape, a square shape, a rectangular shape, a circular shape, a shape with rounded ends or corners, an elongate shape, a longitudinally elongate shape, a circumferentially elongate shape, and/or other suitable sizes and/or shapes. In one example, the window(s) 138 may be elongate and have rounded corners, as depicted in FIG. 57. Other suitable configurations of the size and/or shape of the window 138 are contemplated.

The window 138 may have any suitable configuration configured to allow a physician to view the fixation member(s) 34. In one example, the window(s) 138 may be a(n) cut-out or opening in the outer shaft 44. In another example, the window 138 may be a clear or transparent portion of the outer shaft 44, which may be formed from a same or different material than other portions of the outer shaft 44. Other suitable configurations of the window 138 are contemplated.

This disclosure is, in many respects, only illustrative. Changes may be made in details, particularly in matters of shape, size, and arrangement of steps without exceeding the scope of the disclosure. This may include, to the extent that it is appropriate, the use of any of the features of one example configuration being used in other configurations. The disclosure's scope is, of course, defined in the language in which the appended claims are expressed.

What is claimed is:

1. A fixation member delivery system configured to deliver a plurality of fixation members, the fixation member delivery system comprising:
a handle;
an elongate shaft configured to adjust along a longitudinal axis relative to the handle;
a first elongate member coupled with the elongate shaft, the first elongate member including a plurality of first engagement members disposed along the first elongate member with a distal end region of each of one or more of the first engagement members configured to engage with one fixation member of the plurality of fixation members;
a second elongate member fixed relative to the handle, the second elongate member including a plurality of second engagement members disposed along the second elongate member with a distal end region of each of one or more of the second engagement members configured to engage with one fixation member of the plurality of fixation members, and
an outer shaft extending distally from the handle, the outer shaft defining a shaft lumen;
wherein the elongate shaft, the first elongate member, and the second elongate member extend along the shaft lumen; and
wherein cyclical actuation of the elongate shaft and the first elongate member is configured to incrementally move the plurality of fixation members distally.

2. The delivery system of claim 1, wherein a proximal end of the second elongate member is engaged with the handle.

3. The delivery system of claim 1, wherein the outer shaft has an oval cross-section.

4. The delivery system of claim 1, further comprising:
a filler component extending along the shaft lumen, and
wherein the filler component defines a fixation member lumen configured to receive the plurality of fixation members.

5. The delivery system of claim 4, wherein the filler component defines a channel in communication with the fixation lumen and configured to receive the first elongate member.

6. The delivery system of claim 5, wherein the channel is configured to permit longitudinal movement of the first elongate member and the plurality of first engagement members.

7. The delivery system of claim 4, wherein the filler component defines a channel in communication with the fixation lumen and the channel is configured to receive and fix the second elongate member relative to the filler component.

8. The delivery system of claim 4, wherein the filler component defines a recess configured to receive a second engagement member of the plurality of second engagement members when the second engagement member flexes laterally outward.

9. The delivery system of claim 4, wherein the first elongate member defines an opening configured to receive a first engagement member of the plurality of first engagement members when the first engagement member flexes laterally outward.

10. The delivery system of claim 1, further comprising an upper insert and a lower insert positioned within the shaft lumen of the outer shaft proximate a distal end of the outer shaft;
wherein the upper insert includes a notch configured to receive of portion of the first elongate member therein; and
wherein the lower insert includes a notch configured to receive a portion of the second elongate member therein.

11. The delivery system of claim 1, wherein a distal end region of the outer shaft includes at least one crimp.

12. The delivery system of claim 1, wherein the outer shaft is detachable from the handle via a coupling interface.

13. The delivery system of claim 12, wherein the coupling interface includes a locking mechanism for selectively locking axial movement of the first elongate member relative to the outer shaft.

14. A fixation member delivery system configured to deliver a plurality of fixation members, the fixation member delivery system comprising:
an elongate shaft configured to adjust along a longitudinal axis;
a first elongate member configured to move with the elongate shaft as the elongate shaft adjusts along the longitudinal axis, the first elongate member including a plurality of flexible first engagement members disposed along the first elongate member with a distal end region of each of one or more first engagement members configured to engage one fixation member of the plurality of fixation members; and
a second elongate member fixed relative to the longitudinal axis, the second elongate member including a plurality of flexible second engagement members disposed along the second elongate member with a distal end region of each of one or more second engagement members configured to engage one fixation member of the plurality of fixation members,
wherein the elongate shaft and the first elongate member are configured to advance distally relative to the second elongate member and cause distal end regions of the first engagement members to engage a proximal end of a fixation member of the plurality of fixation members and advance the fixation member distally over a second engagement member of the plurality of second engagement members; and
wherein a distal most second engagement member of the plurality of second engagement members is configured to extend distal of a distal most fixation member of the plurality of fixation members to prevent inadvertent deployment of the distal most fixation member.

15. The delivery system of claim 14, wherein the elongate shaft and the first elongate member are configured to advance proximally relative to the second elongate member and the distal end region of the second engagement members of the plurality of second engagement members engages a proximal end of a fixation member to limit proximal movement of the fixation member.

16. A fixation member delivery system configured to deliver a plurality of fixation members, the fixation member delivery system comprising:
an elongate shaft configured to adjust along a longitudinal axis;
a first elongate member configured to move with the elongate shaft as the elongate shaft adjusts along the longitudinal axis, the first elongate member including a plurality of flexible first engagement members disposed along the first elongate member with a distal end region of each of one or more first engagement members configured to engage one fixation member of the plurality of fixation members; and
a second elongate member fixed relative to the longitudinal axis, the second elongate member including a plurality of flexible second engagement members disposed along the second elongate member with a distal end region of each of one or more second engagement members configured to engage one fixation member of the plurality of fixation members,
wherein the elongate shaft and the first elongate member are configured to advance distally relative to the second elongate member and cause distal end regions of the first engagement members to engage a proximal end of a fixation member of the plurality of fixation members and advance the fixation member distally over a second engagement member of the plurality of second engagement members; and
wherein a distal most first engagement member of the plurality of first engagement members comprises a distal fork configuration configured to receive a second engagement member of the plurality of second engagement members.

17. A fixation member delivery system configured to deliver a plurality of fixation members, the fixation member delivery system comprising:
an elongate shaft configured to adjust along a longitudinal axis;
a first elongate member configured to move with the elongate shaft as the elongate shaft adjusts along the longitudinal axis, the first elongate member including a plurality of flexible first engagement members disposed along the first elongate member with a distal end region of each of one or more first engagement members configured to engage one fixation member of the plurality of fixation members;
a second elongate member fixed relative to the longitudinal axis, the second elongate member including a plurality of flexible second engagement members disposed along the second elongate member with a distal end region of each of one or more second engagement members configured to engage one fixation member of the plurality of fixation members, and
a filler component extending along the first elongate member and the second elongate member;
wherein the elongate shaft and the first elongate member are configured to advance distally relative to the second elongate member and cause distal end regions of the first engagement members to engage a proximal end of a fixation member of the plurality of fixation members and advance the fixation member distally over a second engagement member of the plurality of second engagement members; and
wherein the filler component is configured to fix the second elongate member relative to the longitudinal axis.

18. A fixation member delivery system configured to deliver a plurality of fixation members, the fixation member delivery system comprising:
a handle;
an outer shaft extending distally from the handle, the outer shaft including a shaft lumen;
an elongate shaft configured to reciprocate along a longitudinal axis relative to the handle;
a first elongate member coupled with the elongate shaft, the first elongate member including a plurality of first engagement members disposed along the first elongate member with a distal end region of each of one or more of the first engagement members configured to engage with one fixation member of the plurality of fixation members;
a first filler component extending along the shaft lumen, and
a second filler component extending along the shaft lumen, the second filler component including a plurality openings and a corresponding second engagement member disposed therewithin, each of the second engagement members configured to engage with one fixation member of the plurality of fixation members to prevent proximal retraction thereof;
wherein cyclical actuation of the elongate shaft and the first elongate member is configured to incrementally move the plurality of fixation members distally.

19. The delivery system of claim 18, wherein the plurality of openings are uniformly spaced along the second filler component.

20. The delivery system of claim 18, wherein the first filler component and the second filler component are formed of a non-thermally conductive polymeric material.

21. A method of deploying a fixation member of a plurality of fixation members from a fixation member delivery system, the method comprising:
advancing the plurality of fixation members distally by advancing a first elongate member distally relative to a second elongate member, wherein each of one or more fixation members of the plurality of fixation members engages and flexes an engagement member of a plurality of engagement members extending from the second elongate member;
deploying a distal most fixation member of the plurality of fixation members; and
advancing the first elongate member proximally relative to the second elongate member, wherein each of two or more of the plurality of engagement members extending from the second elongate member engage a fixation member of the plurality of fixation members to limit proximal movement of the fixation member as the first elongate member is advanced proximally;
wherein the fixation members are configured to advance distally through a filler component extending along the first elongate member and the second elongate member.

22. A method of deploying a fixation member of a plurality of fixation members from a fixation member delivery system, the method comprising:
advancing the plurality of fixation members distally by advancing a first elongate member distally relative to a second elongate member, wherein each of one or more fixation members of the plurality of fixation members engages and flexes an engagement member of a plurality of engagement members extending from the second elongate member;
deploying a distal most fixation member of the plurality of fixation members; and
advancing the first elongate member proximally relative to the second elongate member, wherein each of two or more of the plurality of engagement members extending from the second elongate member engage a fixation member of the plurality of fixation members to limit proximal movement of the fixation member as the first elongate member is advanced proximally;
wherein the fixation member comprises an interior prong and a planar proximal surface that is perpendicular to a plane along which the fixation member advances distally.
